SEQUENCE LISTING (142 sequences)

SEQ ID NO: 1 - protein, 72 aa, Artificial Sequence
  Description of Artificial Sequence Synthetic polypeptide
  aa 28-99 of recombinant human IL8 (NP_000575.1)
SAKELRCQCIKTYSKPFHPKFIKELRVIESGPHCANTEIIVKLSDGRELCLDPKENWVQRVVEKFLKRAENS

SEQ ID NO: 2 - protein, 117 aa, Artificial Sequence
  Description of Artificial Sequence Synthetic polypeptide
  STLX3 VH
EVQLVQSGAEVKKAGESLKISCKGSGYSFTSYWIGWVRQMPGKGLEWMGIIYPGDSDTRYSPSFQGQVTISADKSISTAYLQWSSLKASDTAMYYCARDITGNFDYWGQGTLVTVSS

SEQ ID NO: 3 - protein, 108 aa, Artificial Sequence
  Description of Artificial Sequence Synthetic polypeptide
  STLX3 VL
DIQMTQSPSSLSASVGDRVTITCQASQDISNYLNWYQQKPGKAPKVLIYDASNLETGVPSRFSGSGSGTDFTFTISSLQPEDIATYYCQQYDNFPQLTFGGGTKVEIK

SEQ ID NO: 4 - protein, 116 aa, Artificial Sequence
  Description of Artificial Sequence Synthetic polypeptide
  STLX5 VH
QVQLQESGPGLVKPSETLSLTCTVSGGPISSYYWSWIRQPPGKGLEWIGYIYYIGSTDYNPSLKSRVTISKDTSKNQFSLKLSSVTAADTAVYYCARWELHAFDIWGQGTMVTVSS

SEQ ID NO: 5 - protein, 106 aa, Artificial Sequence
  Description of Artificial Sequence Synthetic polypeptide
  STLX5 VL
DIQMTQSPSSLSASVGDRVTITCQASQDISNYLNWYQQKPGKAPKLLIYDASNLETGVPSRFSGSGSGTDFTFTISSLQPEDIATYYCQQYDNLFTFGPGTKVDIK

SEQ ID NO: 6 - protein, 118 aa, Artificial Sequence
  Description of Artificial Sequence Synthetic polypeptide
  STLX14 VH
EVQLVESGGGLVQPGGSLRLSCAASGFTFSSYDMDWVRQATGKGLEWVSTIGTAGDTYYPGSVKGRFTISRENAKNSLYLQMNSLRAGDTAVYYCARGRAPHWYFDLWGRGTLVTVSS

SEQ ID NO: 7 - protein, 107 aa, Artificial Sequence
  Description of Artificial Sequence Synthetic polypeptide
  STLX14 VL
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDVVFGGGTKLTVL

SEQ ID NO: 8 - protein, 117 aa, Artificial Sequence
  Description of Artificial Sequence Synthetic polypeptide
  STLX18 VH
EVQLVESGGGLVQPGGSLRLSCAASGFTFSSYDMNWVRQATGKGLEWVSAIGPAGDTYYPGSVKGRFTISRENAKNSLYLQMNSLRAGDTAVYYCARERWPGYFDLWGRGTLVTVSS

SEQ ID NO: 9 - protein, 108 aa, Artificial Sequence
  Description of Artificial Sequence Synthetic polypeptide
  STLX18 VL
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHVVFGGGTKLTVL

SEQ ID NO: 10 - protein, 117 aa, Artificial Sequence
  Description of Artificial Sequence Synthetic polypeptide
  STLX21 VH
EVQLVQSGAEVKKAGESLKISCKGSGYSFTSYWIGWVRQMPGKGLEWMGIIYPGSSDTRYSPSFQGQVTISADKSISTAYLQWSSLKASDTAMYYCARDITGNFDYWGQGTLVTVSS

SEQ ID NO: 11 - protein, 108 aa, Artificial Sequence; identical to SEQ ID NO: 3
  Description of Artificial Sequence Synthetic polypeptide
  STLX21 VL

SEQ ID NO: 12 - protein, 117 aa, Artificial Sequence
  Description of Artificial Sequence Synthetic polypeptide
  STLX22 VH
EVQLVQSGAEVKKAGESLKISCKGSGYSFTSYWIGWVRQMPGKGLEWMGIIYPGTSDTRYSPSFQGQVTISADKSISTAYLQWSSLKASDTAMYYCARDITGNFDYWGQGTLVTVSS

SEQ ID NO: 13 - protein, 108 aa, Artificial Sequence; identical to SEQ ID NO: 3
  Description of Artificial Sequence Synthetic polypeptide
  STLX22 VL

SEQ ID NO: 14 - protein, 119 aa, Artificial Sequence
  Description of Artificial Sequence Synthetic polypeptide
  STLX31 VH
EVQLVESGGGLVKPGGSLRLSCAASGFTFSSFSMSWVRQAPGKGLEWVSSISSSSSYIDYADSVKGRFTISRDNAKNSLYLQMNSLTAEDTAVYFCARDVGPYWYFDLWGRGTLVTVSS

SEQ ID NO: 15 - protein, 108 aa, Artificial Sequence
  Description of Artificial Sequence Synthetic polypeptide
  STLX31 VL
SYVLTQPPSVSVAPGQTARITCGGNNIGSKSVHWYQQKPGQAPVLVVYDESDRPSGILEGFSGSNLGNTATLTISRVEAGDEADYYCQVWDSSSDHVVFGGGTKLTVL

SEQ ID NO: 16 - protein, 117 aa, Artificial Sequence
  Description of Artificial Sequence Synthetic polypeptide
  STLX35 VH
QVQLVESGGGEVQPGRSLRLSCPASGFTFSSYGMHWVRQAPGKGLEWVALISYDGGNKYYADSVKGRFTISRDNSKNTLYLQMNSLRPEDTAVYYCARDRVGILDYWGQGTLVTVSS

SEQ ID NO: 17 - protein, 105 aa, Artificial Sequence
  Description of Artificial Sequence Synthetic polypeptide
  STLX35 VL
DIQMTQSPSSLSASVGDRVTITCQASQDISNYLNWFQQKPGKAPKLLIYGASNLETGVPSRFSGSGSGTDFTFTISSLQPEDIATYYCQQYDTLTFGGGTKVEIK

SEQ ID NO: 18 - protein, 119 aa, Artificial Sequence
  Description of Artificial Sequence Synthetic polypeptide
  STLX38 VH
QVQLVQSGTEVKKPGASVKVSCKASGYTFTNYYMHWVRQAPGQGLEWMGVINPSGGGTNYAQKFQGRVTMTRDTSTSTVYMELSSLRSEDTAVYYCARDKAPYYGMDVWGQGTTVTVSS

SEQ ID NO: 19 - protein, 108 aa, Artificial Sequence
  Description of Artificial Sequence Synthetic polypeptide
  STLX38 VL
SYVLTQPPSVSVAPGQTARITCGGNKIGSKSVHWYQQKPGQAPVLVVYEDSDRPSGIPERFSGSNSGNTAALTISRVEAGDEADYYCQVWDSSSDHVVFGGGTKLTVL

SEQ ID NO: 20 - protein, 117 aa, Artificial Sequence; identical to SEQ ID NO: 8
  Description of Artificial Sequence Synthetic polypeptide
  STLX41 VH

SEQ ID NO: 21 - protein, 108 aa, Artificial Sequence
  Description of Artificial Sequence Synthetic polypeptide
  STLX41 VL
SYVLTQPPSVSVAPGQTARITCGGNNIGSKSVHWYQQKPGQAPVLVVYDTSDRPSGIPERFSGSNSGNTATLTISRVEAGDEADYYCQVWDSSSDHVVFGGGTKLTVL

SEQ ID NO: 22 - protein, 117 aa, Artificial Sequence; identical to SEQ ID NO: 8
  Description of Artificial Sequence Synthetic polypeptide
  STLX42 VH

SEQ ID NO: 23 - protein, 108 aa, Artificial Sequence
  Description of Artificial Sequence Synthetic polypeptide
  STLX42 VL
SYVLTQPPSVSVAPGQTARITCGGNNIGSKSVHWYQQKPGQAPVLVVYDDNDRPSGIPERFSGSNSGNTATLTISRVEAGDEADYYCQVWDSSSDHVVFGGGTKLTVL

SEQ ID NO: 24 - protein, 117 aa, Artificial Sequence; identical to SEQ ID NO: 8
  Description of Artificial Sequence Synthetic polypeptide
  STLX43 VH

SEQ ID NO: 25 - protein, 108 aa, Artificial Sequence
  Description of Artificial Sequence Synthetic polypeptide
  STLX43 VL
SYVLTQPPSVSVAPGQTARITCGGNNIGSKSVHWYQQKPGQAPVLVVYDDSDRPSGIPERFSGSNSGNTATLTISRVEAGDEADYYCQVWESSSDHVVFGGGTKLTVL

SEQ ID NO: 26 - protein, 117 aa, Artificial Sequence; identical to SEQ ID NO: 8
  Description of Artificial Sequence Synthetic polypeptide
  STLX44 VH

SEQ ID NO: 27 - protein, 108 aa, Artificial Sequence
  Description of Artificial Sequence Synthetic polypeptide
  STLX44 VL
SYVLTQPPSVSVAPGQTARITCGGNNIGSKSVHWYQQKPGQAPVLVVYDDSDRPSGIPERFSGSNSGNTATLTISRVEAGDEADYYCQVWTSSSDHVVFGGGTKLTVL

SEQ ID NO: 28 - protein, 117 aa, Artificial Sequence; identical to SEQ ID NO: 8
  Description of Artificial Sequence Synthetic polypeptide
  STLX45 VH

SEQ ID NO: 29 - protein, 108 aa, Artificial Sequence
  Description of Artificial Sequence Synthetic polypeptide
  STLX45 VL
SYVLTQPPSVSVAPGQTARITCGGNNIGSKSVHWYQQKPGQAPVLVVYDTSDRPSGIPERFSGSNSGNTATLTISRVEAGDEADYYCQVWESSSDHVVFGGGTKLTVL

SEQ ID NO: 30 - protein, 117 aa, Artificial Sequence; identical to SEQ ID NO: 8
  Description of Artificial Sequence Synthetic polypeptide
  STLX46 VH

SEQ ID NO: 31 - protein, 108 aa, Artificial Sequence
  Description of Artificial Sequence Synthetic polypeptide
  STLX46 VL
SYVLTQPPSVSVAPGQTARITCGGNNIGSKSVHWYQQKPGQAPVLVVYDTSDRPSGIPERFSGSNSGNTATLTISRVEAGDEADYYCQVWTSSSDHVVFGGGTKLTVL

SEQ ID NO: 32 - protein, 117 aa, Artificial Sequence; identical to SEQ ID NO: 8
  Description of Artificial Sequence Synthetic polypeptide
  STLX47 VH

SEQ ID NO: 33 - protein, 108 aa, Artificial Sequence
  Description of Artificial Sequence Synthetic polypeptide
  STLX47 VL
SYVLTQPPSVSVAPGQTARITCGGNNIGSKSVHWYQQKPGQAPVLVVYDDNDRPSGIPERFSGSNSGNTATLTISRVEAGDEADYYCQVWESSSDHVVFGGGTKLTVL

SEQ ID NO: 34 - protein, 117 aa, Artificial Sequence; identical to SEQ ID NO: 8
  Description of Artificial Sequence Synthetic polypeptide
  STLX48 VH

SEQ ID NO: 35 - protein, 108 aa, Artificial Sequence
  Description of Artificial Sequence Synthetic polypeptide
  STLX48 VL
SYVLTQPPSVSVAPGQTARITCGGNNIGSKSVHWYQQKPGQAPVLVVYDDNDRPSGIPERFSGSNSGNTATLTISRVEAGDEADYYCQVWTSSSDHVVFGGGTKLTVL

SEQ ID NO: 36 - DNA, 351 bp, Artificial Sequence
  Description of Artificial Sequence Synthetic polynucleotide
  STLX47 VH
gaagtgcagctggttgaatctggcggcggattggttcagcctggcggatctctgagactgtcttgtgccgcctccggcttcaccttctccagctacgatatgaactgggtccgacaggccaccggcaaaggactggaatgggtgtccgctattggccctgccggcgacacatattaccccggctctgtgaagggcagattcaccatcagcagagagaacgccaagaactccctgtacctgcagatgaacagcctgagagccggcgataccgccgtgtactactgtgccagagagagatggcccggctacttcgatctgtggggcagaggaacactggtcaccgtgtctagc

SEQ ID NO: 37 - DNA, 324 bp, Artificial Sequence
  Description of Artificial Sequence Synthetic polynucleotide
  STLX47 VL
tcctacgtgctgacccagcctccttccgtgtctgttgctcctggccagaccgccagaatcacctgtggcggaaacaacatcggctccaagtccgtgcactggtatcagcagaagcctggacaggctcctgtgctggtggtgtacgacgacaacgacagaccctctggcatccctgagagattctccggctccaacagcggcaataccgccacactgaccatctccagagtggaagctggcgacgaggccgactactactgccaagtgtgggagtcctcctccgatcacgtggtgtttggcggcggaacaaagctgacagtgctg

SEQ ID NO: 38 - DNA, 351 bp, Artificial Sequence; identical to SEQ ID NO: 36
  Description of Artificial Sequence Synthetic polynucleotide
  STLX48 VH

SEQ ID NO: 39 - DNA, 324 bp, Artificial Sequence
  Description of Artificial Sequence Synthetic polynucleotide
  STLX48 VL
tcctacgtgctgacccagcctccttccgtgtctgttgctcctggccagaccgccagaatcacctgtggcggaaacaacatcggctccaagtccgtgcactggtatcagcagaagcctggacaggctcctgtgctggtggtgtacgacgacaacgacagaccctctggcatccctgagagattctccggctccaacagcggcaataccgccacactgaccatctccagagtggaagctggcgacgaggccgactactactgccaagtgtggacctcctcctccgatcacgtggtgtttggcggcggaacaaagctgacagtgctg

SEQ ID NO: 40 - protein, 11 aa, Artificial Sequence
  Description of Artificial Sequence Synthetic peptide
  CDR-L1
QASQDISNYLN

SEQ ID NO: 41 - protein, 11 aa, Artificial Sequence
  Description of Artificial Sequence Synthetic peptide
  CDR-L1
GGNNIGSKSVH

SEQ ID NO: 42 - protein, 11 aa, Artificial Sequence
  Description of Artificial Sequence Synthetic peptide
  CDR-L1
GGNKIGSKSVH

SEQ ID NO: 43 - protein, 7 aa, Artificial Sequence
  Description of Artificial Sequence Synthetic peptide
  CDR-L2
DASNLET

SEQ ID NO: 44 - protein, 7 aa, Artificial Sequence
  Description of Artificial Sequence Synthetic peptide
  CDR-L2
DDSDRPS

SEQ ID NO: 45 - protein, 7 aa, Artificial Sequence
  Description of Artificial Sequence Synthetic peptide
  CDR-L2
DESDRPS

SEQ ID NO: 46 - protein, 7 aa, Artificial Sequence
  Description of Artificial Sequence Synthetic peptide
  CDR-L2
GASNLET

SEQ ID NO: 47 - protein, 7 aa, Artificial Sequence
  Description of Artificial Sequence Synthetic peptide
  CDR-L2
EDSDRPS

SEQ ID NO: 48 - protein, 7 aa, Artificial Sequence
  Description of Artificial Sequence Synthetic peptide
  CDR-L2
DTSDRPS

SEQ ID NO: 49 - protein, 7 aa, Artificial Sequence
  Description of Artificial Sequence Synthetic peptide
  CDR-L2
DDNDRPS

SEQ ID NO: 50 - protein, 10 aa, Artificial Sequence
  Description of Artificial Sequence Synthetic peptide
  CDR-L3
QQYDNFPQLT

SEQ ID NO: 51 - protein, 8 aa, Artificial Sequence
  Description of Artificial Sequence Synthetic peptide
  CDR-L3
QQYDNLFT

SEQ ID NO: 52 - protein, 10 aa, Artificial Sequence
  Description of Artificial Sequence Synthetic peptide
  CDR-L3
QVWDSSSDVV

SEQ ID NO: 53 - protein, 11 aa, Artificial Sequence
  Description of Artificial Sequence Synthetic peptide
  CDR-L3
QVWDSSSDHVV

SEQ ID NO: 54 - protein, 7 aa, Artificial Sequence
  Description of Artificial Sequence Synthetic peptide
  CDR-L3
QQYDTLT

SEQ ID NO: 55 - protein, 11 aa, Artificial Sequence
  Description of Artificial Sequence Synthetic peptide
  CDR-L3
QVWESSSDHVV

SEQ ID NO: 56 - protein, 11 aa, Artificial Sequence
  Description of Artificial Sequence Synthetic peptide
  CDR-L3
QVWTSSSDHVV

SEQ ID NO: 57 - protein, 5 aa, Artificial Sequence
  Description of Artificial Sequence Synthetic peptide
  CDR-H1
SYWIG

SEQ ID NO: 58 - protein, 5 aa, Artificial Sequence
  Description of Artificial Sequence Synthetic peptide
  CDR-H1
SYYWS

SEQ ID NO: 59 - protein, 5 aa, Artificial Sequence
  Description of Artificial Sequence Synthetic peptide
  CDR-H1
SYDMD

SEQ ID NO: 60 - protein, 5 aa, Artificial Sequence
  Description of Artificial Sequence Synthetic peptide
  CDR-H1
SYDMN

SEQ ID NO: 61 - protein, 5 aa, Artificial Sequence
  Description of Artificial Sequence Synthetic peptide
  CDR-H1
SFSMS

SEQ ID NO: 62 - protein, 5 aa, Artificial Sequence
  Description of Artificial Sequence Synthetic peptide
  CDR-H1
SYGMH

SEQ ID NO: 63 - protein, 5 aa, Artificial Sequence
  Description of Artificial Sequence Synthetic peptide
  CDR-H1
NYYMH

SEQ ID NO: 64 - protein, 17 aa, Artificial Sequence
  Description of Artificial Sequence Synthetic peptide
  CDR-H2
IIYPGDSDTRYSPSFQG

SEQ ID NO: 65 - protein, 16 aa, Artificial Sequence
  Description of Artificial Sequence Synthetic peptide
  CDR-H2
YIYYIGSTDYNPSLKS

SEQ ID NO: 66 - protein, 16 aa, Artificial Sequence
  Description of Artificial Sequence Synthetic peptide
  CDR-H2
TIGTAGDTYYPGSVKG

SEQ ID NO: 67 - protein, 16 aa, Artificial Sequence
  Description of Artificial Sequence Synthetic peptide
  CDR-H2
AIGPAGDTYYPGSVKG

SEQ ID NO: 68 - protein, 17 aa, Artificial Sequence
  Description of Artificial Sequence Synthetic peptide
  CDR-H2
IIYPGSSDTRYSPSFQG

SEQ ID NO: 69 - protein, 17 aa, Artificial Sequence
  Description of Artificial Sequence Synthetic peptide
  CDR-H2
IIYPGTSDTRYSPSFQG

SEQ ID NO: 70 - protein, 17 aa, Artificial Sequence
  Description of Artificial Sequence Synthetic peptide
  CDR-H2
SISSSSSYIDYADSVKG

SEQ ID NO: 71 - protein, 17 aa, Artificial Sequence
  Description of Artificial Sequence Synthetic peptide
  CDR-H2
LISYDGGNKYYADSVKG

SEQ ID NO: 72 - protein, 17 aa, Artificial Sequence
  Description of Artificial Sequence Synthetic peptide
  CDR-H2
VINPSGGGTNYAQKFQG

SEQ ID NO: 73 - protein, 8 aa, Artificial Sequence
  Description of Artificial Sequence Synthetic peptide
  CDR-H3
DITGNFDY

SEQ ID NO: 74 - protein, 8 aa, Artificial Sequence
  Description of Artificial Sequence Synthetic peptide
  CDR-H3
WELHAFDI

SEQ ID NO: 75 - protein, 10 aa, Artificial Sequence
  Description of Artificial Sequence Synthetic peptide
  CDR-H3
GRAPHWYFDL

SEQ ID NO: 76 - protein, 9 aa, Artificial Sequence
  Description of Artificial Sequence Synthetic peptide
  CDR-H3
ERWPGYFDL

SEQ ID NO: 77 - protein, 10 aa, Artificial Sequence
  Description of Artificial Sequence Synthetic peptide
  CDR-H3
DVGPYWYFDL

SEQ ID NO: 78 - protein, 8 aa, Artificial Sequence
  Description of Artificial Sequence Synthetic peptide
  CDR-H3
DRVGILDY

SEQ ID NO: 79 - protein, 10 aa, Artificial Sequence
  Description of Artificial Sequence Synthetic peptide
  CDR-H3
DKAPYYGMDV

SEQ ID NO: 80 - protein, 6 aa, Artificial Sequence
  Description of Artificial Sequence Synthetic peptide
  CDR-L1
QDISNY

SEQ ID NO: 81 - protein, 6 aa, Artificial Sequence
  Description of Artificial Sequence Synthetic peptide
  CDR-L1
NIGSKS

SEQ ID NO: 82 - protein, 6 aa, Artificial Sequence
  Description of Artificial Sequence Synthetic peptide
  CDR-L1
KIGSKS

SEQ ID NO: 83 - protein, 3 aa, Artificial Sequence
  Description of Artificial Sequence Synthetic peptide
  CDR-L2
DAS

SEQ ID NO: 84 - protein, 3 aa, Artificial Sequence
  Description of Artificial Sequence Synthetic peptide
  CDR-L2
DDS

SEQ ID NO: 85 - protein, 3 aa, Artificial Sequence
  Description of Artificial Sequence Synthetic peptide
  CDR-L2
DES

SEQ ID NO: 86 - protein, 3 aa, Artificial Sequence
  Description of Artificial Sequence Synthetic peptide
  CDR-L2
GAS

SEQ ID NO: 87 - protein, 3 aa, Artificial Sequence
  Description of Artificial Sequence Synthetic peptide
  CDR-L2
EDS

SEQ ID NO: 88 - protein, 3 aa, Artificial Sequence
  Description of Artificial Sequence Synthetic peptide
  CDR-L2
DTS

SEQ ID NO: 89 - protein, 3 aa, Artificial Sequence
  Description of Artificial Sequence Synthetic peptide
  CDR-L2
DDN

SEQ ID NO: 90 - protein, 8 aa, Artificial Sequence
  Description of Artificial Sequence Synthetic peptide
  CDR-H1
GYSFTSYW

SEQ ID NO: 91 - protein, 8 aa, Artificial Sequence
  Description of Artificial Sequence Synthetic peptide
  CDR-H1
GGPISSYY

SEQ ID NO: 92 - protein, 8 aa, Artificial Sequence
  Description of Artificial Sequence Synthetic peptide
  CDR-H1
GFTFSSYD

SEQ ID NO: 93 - protein, 8 aa, Artificial Sequence
  Description of Artificial Sequence Synthetic peptide
  CDR-H1
GFTFSSYD

SEQ ID NO: 94 - protein, 8 aa, Artificial Sequence
  Description of Artificial Sequence Synthetic peptide
  CDR-H1
GFTFSSFS

SEQ ID NO: 95 - protein, 8 aa, Artificial Sequence
  Description of Artificial Sequence Synthetic peptide
  CDR-H1
GFTFSSYG

SEQ ID NO: 96 - protein, 8 aa, Artificial Sequence
  Description of Artificial Sequence Synthetic peptide
  CDR-H1
GYTFTNYY

SEQ ID NO: 97 - protein, 8 aa, Artificial Sequence
  Description of Artificial Sequence Synthetic peptide
  CDR-H2
IYPGDSDT

SEQ ID NO: 98 - protein, 7 aa, Artificial Sequence
  Description of Artificial Sequence Synthetic peptide
  CDR-H2
IYYIGST

SEQ ID NO: 99 - protein, 7 aa, Artificial Sequence
  Description of Artificial Sequence Synthetic peptide
  CDR-H2
IGTAGDT

SEQ ID NO: 100 - protein, 7 aa, Artificial Sequence
  Description of Artificial Sequence Synthetic peptide
  CDR-H2
IGPAGDT

SEQ ID NO: 101 - protein, 8 aa, Artificial Sequence
  Description of Artificial Sequence Synthetic peptide
  CDR-H2
IYPGSSDT

SEQ ID NO: 102 - protein, 8 aa, Artificial Sequence
  Description of Artificial Sequence Synthetic peptide
  CDR-H2
IYPGTSDT

SEQ ID NO: 103 - protein, 8 aa, Artificial Sequence
  Description of Artificial Sequence Synthetic peptide
  CDR-H2
ISSSSSYI

SEQ ID NO: 104 - protein, 8 aa, Artificial Sequence
  Description of Artificial Sequence Synthetic peptide
  CDR-H2
ISYDGGNK

SEQ ID NO: 105 - protein, 8 aa, Artificial Sequence
  Description of Artificial Sequence Synthetic peptide
  CDR-H2
INPSGGGT

SEQ ID NO: 106 - protein, 10 aa, Artificial Sequence
  Description of Artificial Sequence Synthetic peptide
  CDR-H3
ARDITGNFDY

SEQ ID NO: 107 - protein, 10 aa, Artificial Sequence
  Description of Artificial Sequence Synthetic peptide
  CDR-H3
ARWELHAFDI

SEQ ID NO: 108 - protein, 12 aa, Artificial Sequence
  Description of Artificial Sequence Synthetic peptide
  CDR-H3
ARGRAPHWYFDL

SEQ ID NO: 109 - protein, 11 aa, Artificial Sequence
  Description of Artificial Sequence Synthetic peptide
  CDR-H3
ARERWPGYFDL

SEQ ID NO: 110 - protein, 12 aa, Artificial Sequence
  Description of Artificial Sequence Synthetic peptide
  CDR-H3
ARDVGPYWYFDL

SEQ ID NO: 111 - protein, 10 aa, Artificial Sequence
  Description of Artificial Sequence Synthetic peptide
  CDR-H3
ARDRVGILDY

SEQ ID NO: 112 - protein, 12 aa, Artificial Sequence
  Description of Artificial Sequence Synthetic peptide
  CDR-H3
ARDKAPYYGMDV

SEQ ID NO: 113 - DNA, 351 bp, Artificial Sequence
  Description of Artificial Sequence Synthetic polynucleotide
  STLX3 VH
gaagttcagctggttcagtctggcgccgaagtgaagaaggctggcgagtccctgaagatctcctgcaaaggctccggctactccttcacctcctactggatcggctgggtccgacagatgcctggcaaaggcttggagtggatgggcatcatctaccccggcgactctgataccagatactcccctagcttccagggccaagtgaccatctccgccgacaagtctatctccaccgcctacctgcagtggtcctctctgaaggcttctgacaccgccatgtactactgcgccagagacatcaccggcaacttcgactattggggccagggcacactggtcaccgtgtcctct

SEQ ID NO: 114 - DNA, 324 bp, Artificial Sequence
  Description of Artificial Sequence Synthetic polynucleotide
  STLX3 VL
gacatccagatgacccagtctccatcctctctgtccgcctctgtgggcgacagagtgaccattacctgtcaggccagccaggacatctccaactacctgaactggtatcagcagaagcccggcaaggcccctaaggtgctgatctacgatgcctccaacctggaaaccggcgtgccctctagattctccggctctggctctggcaccgactttacctttacaatctccagcctgcagcctgaggatatcgccacctactactgccagcagtacgacaacttccctcagctgacctttggcggaggcaccaaggtggaaatcaag

SEQ ID NO: 115 - DNA, 348 bp, Artificial Sequence
  Description of Artificial Sequence Synthetic polynucleotide
  STLX5 VH
caggttcagctgcaagagtctggccctggcctggtcaagccttccgaaacactgtctctgacctgcaccgtgtctggcggccctatctcctcctactactggtcctggatcagacagcctcctggcaaaggcctggaatggatcggctacatctactacatcggctccaccgactacaaccccagcctgaagtccagagtgaccatctccaaggacacctccaagaaccagttctccctgaagctgtcctccgtgaccgctgctgataccgccgtgtactactgtgccagatgggagctgcacgccttcgatatctggggccagggcacaatggtcaccgtgtcctct

SEQ ID NO: 116 - DNA, 318 bp, Artificial Sequence
  Description of Artificial Sequence Synthetic polynucleotide
  STLX5 VL
gacatccagatgacccagtctccatcctctctgtccgcctctgtgggcgacagagtgaccattacctgtcaggccagccaggacatctccaactacctgaactggtatcagcagaagcccggcaaggcccctaagctgctgatctacgatgcctccaacctggaaaccggcgtgccctctagattctccggctctggctctggcaccgactttacctttacaatctccagcctgcagcctgaggatatcgccacctactactgccagcagtacgacaacctgttcaccttcggacccggcaccaaggtggacatcaaa

SEQ ID NO: 117 - DNA, 354 bp, Artificial Sequence
  Description of Artificial Sequence Synthetic polynucleotide
  STLX14 VH
gaggtgcagctggtggagtctgggggaggcttggtacagcctggggggtccctgagactctcctgtgcagcctctggattcaccttcagtagctacgacatggactgggtccgccaagctacaggaaaaggtctggagtgggtctcaactattggtactgctggtgacacatactatccaggctccgtgaagggccgattcaccatctccagagaaaatgccaagaactccttgtatcttcaaatgaacagcctgagagccggggacacggctgtgtattactgtgcaagagggagggctcctcactggtacttcgatctctggggccgtggcaccctggtcactgtctcctca

SEQ ID NO: 118 - DNA, 321 bp, Artificial Sequence
  Description of Artificial Sequence Synthetic polynucleotide
  STLX14 VL
tcctatgtgctgactcagccaccctcggtgtcagtggccccaggacagacggccaggattacctgtgggggaaacaacattggaagtaaaagtgtgcactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatgtggtattcggcggagggaccaagctgaccgtccta

SEQ ID NO: 119 - DNA, 351 bp, Artificial Sequence; identical to SEQ ID NO: 36
  Description of Artificial Sequence Synthetic polynucleotide
  STLX18 VH

SEQ ID NO: 120 - DNA, 324 bp, Artificial Sequence
  Description of Artificial Sequence Synthetic polynucleotide
  STLX18 VL
tcctacgtgctgacccagcctccttccgtgtctgttgctcctggccagaccgccagaatcacctgtggcggaaacaacatcggctccaagtccgtgcactggtatcagcagaagcctggacaggctcctgtgctggtggtgtacgacgactccgatagaccctctggcatccctgagagattctccggctccaacagcggcaataccgccacactgaccatctccagagtggaagctggcgacgaggccgactactactgccaagtgtgggactcctcctccgatcacgtggtgtttggcggcggaacaaagctgacagtgctg

SEQ ID NO: 121 - DNA, 351 bp, Artificial Sequence
  Description of Artificial Sequence Synthetic polynucleotide
  STLX21 VH
gaagttcagctggttcagtctggcgccgaagtgaagaaggctggcgagtccctgaagatctcctgcaaaggctccggctactccttcacctcctactggatcggctgggtccgacagatgcctggcaaaggcttggagtggatgggcatcatctaccccggctcctctgataccagatactcccctagcttccagggccaagtgaccatctccgccgacaagtctatctccaccgcctacctgcagtggtcctctctgaaggcttctgacaccgccatgtactactgcgccagagacatcaccggcaacttcgactattggggccagggcacactggtcaccgtttctagt

SEQ ID NO: 122 - DNA, 324 bp, Artificial Sequence; identical to SEQ ID NO: 114
  Description of Artificial Sequence Synthetic polynucleotide
  STLX21 VL

SEQ ID NO: 123 - DNA, 351 bp, Artificial Sequence
  Description of Artificial Sequence Synthetic polynucleotide
  STLX22 VH
gaagttcagctggttcagtctggcgccgaagtgaagaaggctggcgagtccctgaagatctcctgcaaaggctccggctactccttcacctcctactggatcggctgggtccgacagatgcctggcaaaggcttggagtggatgggcatcatctaccccggcacctctgataccagatactcccctagcttccagggccaagtgaccatctccgccgacaagtctatctccaccgcctacctgcagtggtcctctctgaaggcttctgacaccgccatgtactactgcgccagagacatcaccggcaacttcgactattggggccagggcacactggtcaccgtttctagt

SEQ ID NO: 124 - DNA, 324 bp, Artificial Sequence; identical to SEQ ID NO: 114
  Description of Artificial Sequence Synthetic polynucleotide
  STLX22 VL

SEQ ID NO: 125 - DNA, 357 bp, Artificial Sequence
  Description of Artificial Sequence Synthetic polynucleotide
  STLX31 VH
gaggttcagctggtggagtctgggggaggcctggtcaagcctggggggtccctgagactctcctgtgcagcctctggattcaccttcagtagctttagcatgagctgggtccgccaggctccagggaaggggctggagtgggtctcatccataagtagtagtagtagttacatagactacgcagactcagtgaagggccgattcaccatctccagagacaacgccaagaactcactgtatctgcaaatgaacagcctgacagccgaggacacggctgtgtatttctgtgcgagagatgtaggcccctattggtacttcgatctctggggccgtggcaccctggtcactgtctcctca

SEQ ID NO: 126 - DNA, 324 bp, Artificial Sequence
  Description of Artificial Sequence Synthetic polynucleotide
  STLX31 VL
tcctatgtgctgactcagccaccctcggtgtcagtggccccaggacagacggccaggattacctgtgggggaaacaacattggaagtaaaagtgtgcattggtaccagcagaagccaggccaggcccctgtgttggtagtttatgatgaaagcgaccggccttcagggatccttgagggattttcgggttccaatttggggaacacggccaccctgaccatcagcagggtcgaagccggggatgaggccgactattactgtcaggtgtgggatagtagtagtgatcatgtggtattcggcggagggaccaagctgaccgtccta

SEQ ID NO: 127 - DNA, 351 bp, Artificial Sequence
  Description of Artificial Sequence Synthetic polynucleotide
  STLX35 VH
caggtgcagctggtggagtctgggggaggcgaggtccagcctgggaggtccctgagactctcctgtccagcctctggattcaccttcagtagctatggcatgcactgggtccgccaggctccaggcaaggggctggagtgggtggcacttatatcatatgatggaggtaataaatactatgcagactccgtgaagggccgattcaccatctccagagacaattccaagaacacgctgtatctgcaaatgaacagcctgagacctgaggacacggctgtatattactgtgcgagagatagagtggggatccttgactattggggccagggaaccctggtcaccgtctcctca

SEQ ID NO: 128 - DNA, 315 bp, Artificial Sequence
  Description of Artificial Sequence Synthetic polynucleotide
  STLX35 VL
gacatccagatgacccagtctccatcctccctgtctgcatctgtaggagacagagtcaccatcacttgccaggcgagtcaggacattagcaactatttgaattggtttcagcagaaaccagggaaagcccctaagctcctgatctacggtgcatccaatttggaaacaggggtcccatcaaggttcagtggaagtggatctgggacagattttactttcaccatcagcagcctgcagcctgaagatattgcaacatattactgtcaacagtatgataccctcactttcggcggagggaccaaggtggagatcaaa

SEQ ID NO: 129 - DNA, 357 bp, Artificial Sequence
  Description of Artificial Sequence Synthetic polynucleotide
  STLX38 VH
caggttcagctggtgcagtctggcaccgaagtgaagaaacctggcgcctccgtgaaggtgtcctgcaaggcttctggctacacctttaccaactactacatgcactgggtccgacaggcccctggacaaggattggaatggatgggcgtgatcaacccttctggcggaggcaccaattacgcccagaaattccagggcagagtgaccatgaccagagacacctccaccagcaccgtgtacatggaactgtccagcctgagatccgaggacaccgccgtgtactactgcgccagagataaggccccttactacggcatggatgtgtggggccagggcaccacagtgacagtgtcctct

SEQ ID NO: 130 - DNA, 324 bp, Artificial Sequence
  Description of Artificial Sequence Synthetic polynucleotide
  STLX38 VL
tcctacgtgctgacccagcctccttccgtgtctgttgctcctggccagaccgccagaatcacctgtggcggaaacaagatcggctccaagtccgtgcactggtatcagcagaagcctggacaggctcctgtgctggtggtgtacgaggactccgatagaccctctggcatccctgagagattctccggctccaacagcggcaataccgccgctctgaccatctccagagttgaggctggcgacgaggccgactactactgccaagtgtgggactcctcctccgatcacgtggtgtttggcggcggaacaaagctgacagtgctg

SEQ ID NO: 131 - DNA, 351 bp, Artificial Sequence; identical to SEQ ID NO: 36
  Description of Artificial Sequence Synthetic polynucleotide
  STLX41 VH

SEQ ID NO: 132 - DNA, 324 bp, Artificial Sequence
  Description of Artificial Sequence Synthetic polynucleotide
  STLX41 VL
tcctacgtgctgacccagcctccttccgtgtctgttgctcctggccagaccgccagaatcacctgtggcggaaacaacatcggctccaagtccgtgcactggtatcagcagaagcctggacaggctcctgtgctggtggtgtacgacacctccgatagaccctctggcatccctgagagattctccggctccaacagcggcaataccgccacactgaccatctccagagtggaagctggcgacgaggccgactactactgccaagtgtgggactcctcctccgatcacgtggtgtttggcggcggaacaaagctgacagtgctg

SEQ ID NO: 133 - DNA, 351 bp, Artificial Sequence; identical to SEQ ID NO: 36
  Description of Artificial Sequence Synthetic polynucleotide
  STLX42 VH

SEQ ID NO: 134 - DNA, 324 bp, Artificial Sequence
  Description of Artificial Sequence Synthetic polynucleotide
  STLX42 VL
tcctacgtgctgacccagcctccttccgtgtctgttgctcctggccagaccgccagaatcacctgtggcggaaacaacatcggctccaagtccgtgcactggtatcagcagaagcctggacaggctcctgtgctggtggtgtacgacgacaacgacagaccctctggcatccctgagagattctccggctccaacagcggcaataccgccacactgaccatctccagagtggaagctggcgacgaggccgactactactgccaagtgtgggactcctcctccgatcacgtggtgtttggcggcggaacaaagctgacagtgctg

SEQ ID NO: 135 - DNA, 351 bp, Artificial Sequence; identical to SEQ ID NO: 36
  Description of Artificial Sequence Synthetic polynucleotide
  STLX43 VH

SEQ ID NO: 136 - DNA, 324 bp, Artificial Sequence
  Description of Artificial Sequence Synthetic polynucleotide
  STLX43 VL
tcctacgtgctgacccagcctccttccgtgtctgttgctcctggccagaccgccagaatcacctgtggcggaaacaacatcggctccaagtccgtgcactggtatcagcagaagcctggacaggctcctgtgctggtggtgtacgacgactccgatagaccctctggcatccctgagagattctccggctccaacagcggcaataccgccacactgaccatctccagagtggaagctggcgacgaggccgactactactgccaagtgtgggagtcctcctccgatcacgtggtgtttggcggcggaacaaagctgacagtgctg

SEQ ID NO: 137 - DNA, 351 bp, Artificial Sequence; identical to SEQ ID NO: 36
  Description of Artificial Sequence Synthetic polynucleotide
  STLX44 VH

SEQ ID NO: 138 - DNA, 324 bp, Artificial Sequence
  Description of Artificial Sequence Synthetic polynucleotide
  STLX44 VL
tcctacgtgctgacccagcctccttccgtgtctgttgctcctggccagaccgccagaatcacctgtggcggaaacaacatcggctccaagtccgtgcactggtatcagcagaagcctggacaggctcctgtgctggtggtgtacgacgactccgatagaccctctggcatccctgagagattctccggctccaacagcggcaataccgccacactgaccatctccagagtggaagctggcgacgaggccgactactactgccaagtgtggacctcctcctccgatcacgtggtgtttggcggcggaacaaagctgacagtgctg

SEQ ID NO: 139 - DNA, 351 bp, Artificial Sequence; identical to SEQ ID NO: 36
  Description of Artificial Sequence Synthetic polynucleotide
  STLX45 VH

SEQ ID NO: 140 - DNA, 324 bp, Artificial Sequence
  Description of Artificial Sequence Synthetic polynucleotide
  STLX45 VL
tcctacgtgctgacccagcctccttccgtgtctgttgctcctggccagaccgccagaatcacctgtggcggaaacaacatcggctccaagtccgtgcactggtatcagcagaagcctggacaggctcctgtgctggtggtgtacgacacctctgatagaccctctggcatccctgagagattctccggctccaacagcggcaataccgccacactgaccatctccagagtggaagctggcgacgaggccgactactactgccaagtgtgggagtcctcctccgatcacgtggtgtttggcggcggaacaaagctgacagtgctg

SEQ ID NO: 141 - DNA, 351 bp, Artificial Sequence; identical to SEQ ID NO: 36
  Description of Artificial Sequence Synthetic polynucleotide
  STLX46 VH

SEQ ID NO: 142 - DNA, 324 bp, Artificial Sequence
  Description of Artificial Sequence Synthetic polynucleotide
  STLX46 VL
tcctacgtgctgacccagcctccttccgtgtctgttgctcctggccagaccgccagaatcacctgtggcggaaacaacatcggctccaagtccgtgcactggtatcagcagaagcctggacaggctcctgtgctggtggtgtacgacacctctgatagaccctctggcatccctgagagattctccggctccaacagcggcaataccgccacactgaccatctccagagtggaagctggcgacgaggccgactactactgccaagtgtggacctcctcctccgatcacgtggtgtttggcggcggaacaaagctgacagtgctg

(19) United States
(12) Patent Application Publication
(10) Pub. No.: US 2023/0416355 A1
(43) Pub. Date: Dec. 28, 2023

(54) IL-8 ANTIBODIES AND METHODS OF USE THEREOF

(71) Applicant: STELEXIS THERAPEUTICS, LLC, New York, NY (US)

(72) Inventors: Keren Paz, Tenafly, NJ (US); Nicole Covino, Brooklyn, NY (US); Yasumi Nakayama, Astoria, NY (US); Patrick James Doyle, New York, NY (US); Carmine Carpenito, Hartsdale, NY (US); Lynn Biderman, New York, NY (US); Bozena Bugaj-Gaweda, Westwood, NJ (US); John Andrew Lippincott, San Diego, CA (US); Dana Yen Mei Duey, San Diego, CA (US)

(73) Assignee: STELEXIS THERAPEUTICS, LLC, New York, NY (US)

(21) Appl. No.: 18/019,164

(22) PCT Filed: Aug. 5, 2021
(86) PCT No.: PCT/US2021/044597

§ 371 Date: Feb. 1, 2023

Related U.S. Application Data
(60) Provisional application No. 63/061,857, filed on Aug. 6, 2020.

(51) Int. Cl. C07K 16/24; A61P 35/00; A61P 29/00

(52) U.S. Cl. CPC C07K 16/244; A61K 2039/505; A61P 29/00; A61P 35/00

(57) ABSTRACT

Described herein are anti-IL-8 antibodies, compositions thereof, and methods of use thereof. The anti-IL-8 antibodies N would be useful in methods of inhibiting tumor formation or growth or a combination thereof. Described herein are also methods of using the anti-IL-8 antibodies for methods of treating a disease, wherein diseases may include cancer, a tumor, or a viral infection.

### SEQUENCE LISTING STATEMENT

The instant application contains a Sequence Listing which has been submitted electronically in ASCII format and is hereby incorporated by reference in its entirety. Said ASCII copy, created on Aug. 4, 2021, is named P-585889-PC_SL.txt and is 80,071 bytes in size.

### FIELD OF DISCLOSURE

Disclosed herein are IL-8 antibodies and uses thereof for treating disease, for example but not limited to cancer or diseases associated with a viral infection, or diseases associated with inflammation.

### BACKGROUND

IL-8 is a potent proinflammatory cytokine, secreted by various cells, primarily macrophages. It binds the seven transmembrane G-protein-coupled receptors (GPCRs), CXCR1 and CXCR2, on the cell-surface of neutrophils and monocytes, and impacts their activation and migration to infected areas.

A growing body of evidence suggests a role of IL-8 signaling in cancer, in both hematological malignancies and solid tumors. Studies have shown that IL-8 is critical for survival, invasion, and proliferation of cancer cells and may be an important regulatory of cancer stem cell activity. In fact, IL-8 is frequently overexpressed in many human cancers and increased IL-8 expression has been correlated with poor prognosis of a subject suffering from an IL-8 expressing cancer. Studies have shown upregulation of IL-8 genes in acute myeloid leukemia (AML) and myelodysplastic syndromes (MDS).

IL-8 has been observed to promote cancer progression by influencing the tumor microenvironment (TME), including for example recruiting neutrophils and myeloid-derived suppressor cells to the TME. In addition, IL-8 is known to function in both paracrine and autocrine modes within the TME. Interestingly, decreased presence of IL-8 receptor CXCR2 on a tumor cell surface prolonged survival of the cancer patient and inhibited tumor angiogenesis in certain cancers.

Thus, targets for treating an IL-8 expressing cancers or tumors include the cancer and tumor cells themselves, and the tumor microenvironment.

Further, IL-8 is known as neutrophil chemotactic factor, inducing chemotaxis in target cells, for example but not limited to neutrophils, wherein the presence of IL-8 may be involved in the migration of these cells towards sites of viral infection. There is increasing evidence that IL-8 plays a role in viral infection leading to increased cytokine release syndrome and cytokine storm during viral infections.

There remains a need to provide therapeutic molecule that targets the negative effects of IL-8 in cancer and viral infections. The anti-IL-8 antibodies provided herein address this need by targeting cancer cells expressing IL-8 and or expressing IL-8 receptors, by regulating activation and or migration of cells, for example neutrophils.

### SUMMARY

In one aspect, disclosed herein is an isolated anti-IL-8 antibody comprising an antibody antigen-binding domain comprising a heavy chain variable region (VH) and a light chain variable region (VL), wherein the amino acid sequences of a VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35.

In one aspect, disclosed herein is a composition comprising an isolated anti-IL-8 antibody comprising an antibody antigen-binding domain comprising a heavy chain variable region (VH) and a light chain variable region (VL), wherein the amino acid sequences of a VH VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35; and a pharmaceutically acceptable carrier.

In one aspect, disclosed herein is an isolated polynucleotide sequence encoding a heavy chain variable region (VH) of an anti-IL-8 antibody and a light chain variable region (VL) of the anti-IL-8 antibody, wherein the amino acid sequence of the VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35.

In a related aspect, a polynucleotide sequence comprises two polynucleotide sequences, a first polynucleotide sequence encoding the VH of the anti-IL-8 antibody and a second polynucleotide sequence encoding the VL of the anti-IL-8 antibody.

In one aspect, disclosed herein is a vector comprising the polynucleotide sequence encoding an anti-IL-8 antibody.

In one aspect, disclosed herein is a host cell comprising a vector comprising the polynucleotide sequence encoding an anti-IL-8 antibody.

In one aspect, disclosed herein is a method of producing an anti-IL-8 antibody comprising a heavy chain variable region (VH) and a light chain variable region (VH), said method comprises the step of culturing the host cell of claim 12 under conditions conducive to expressing said vector in said host cell, and expressing said polynucleotide sequences comprised in said vector, thereby producing the anti-IL-8 antibody comprising a VH and a VL.

In one aspect, disclosed herein is an isolated anti-IL-8 antibody having complementarity determining region (CDR) sequences as set forth in Table 1F, wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, wherein said HCDR1, HCDR2, HCDR3, LCDR1, LCDR2 and LCDR3 for each of said antibody comprise the amino acid sequences as set forth in Table 1F.

In one aspect, disclosed herein is a composition comprising an isolated anti-IL-8 antibody having complementarity determining region (CDR) sequences as set forth in Table 1F, wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, wherein said HCDR1, HCDR2, HCDR3, LCDR1, LCDR2 and LCDR3 for each of said antibody comprise the amino acid sequences as set forth in Table 1F; and a pharmaceutically acceptable carrier.

In one aspect, disclosed herein is an isolated polynucleotide sequence encoding a heavy chain variable region (VH) of an anti-IL-8 antibody comprising the complementarity determining regions (HCDR) of said VH as set forth in Table 1F and a light chain variable region (VL) of an anti-IL-8 antibody comprising the complementarity determining regions (LCDR) of said VL as set forth in Table 1F, wherein said heavy chain variable region comprises heavy chain complementarity determining region (HCDR) 1, HCDR2 and HCDR3, and said light chain variable region comprises light chain complementarity determining region (LCDR) 1, LCDR2 and LCDR3, wherein said HCDR1, HCDR2, HCDR3, LCDR1, LCDR2 and LCDR3 for each of said VH and CL comprise the amino acid sequences as set forth in Table 1F.

In a related aspect, a polynucleotide sequence comprises two polynucleotide sequences, a first polynucleotide sequence encoding the VH of the anti-IL-8 antibody and a second polynucleotide sequence encoding the VL of the anti-IL-8 antibody.

In one aspect, disclosed herein is an isolated polynucleotide sequence encoding a heavy chain variable region (VH) of an anti-IL-8 antibody and a light chain variable region (VL) of the anti-IL-8 antibody, wherein the polynucleotide sequences encoding the VH-VL pair are selected from the paired sequences set forth in SEQ ID NOs: 113 and 114, SEQ ID NOs: 115 and 116, SEQ ID NOs: 117 and 118, SEQ ID NOs: 119 and 120, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39.

In a related aspect, a polynucleotide sequence comprises two polynucleotide sequences, a first polynucleotide sequence encoding the VH of the anti-IL-8 antibody and a second polynucleotide sequence encoding the VL of the anti-IL-8 antibody. In another related aspect, disclosed herein is a vector comprising a polynucleotide sequence encoding a heavy chain variable region (VH) of an anti-IL-8 antibody and a light chain variable region (VL) of the anti-IL-8 antibody.

In a related aspect, an antibody disclosed herein comprises an IgG, an Fv, an scFv, an Fab, an F(ab′)2, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody. In a further related aspect, an IgG comprises an IgG1, IgG2, IgG3, or an IgG4.

In one aspect, disclosed herein is a host cell comprising a vector comprising a polynucleotide sequence encoding an anti-IL-8 antibody VH or VL, or combination thereof.

In one aspect, disclosed herein is a method of producing an anti-IL-8 antibody comprising a heavy chain variable region (VH) and a light chain variable region (VH), said method comprises the step of culturing a host cell under conditions conducive to expressing said vector in said host cell, and expressing said polynucleotide sequences comprised in said vector, thereby producing the anti-IL-8 antibody comprising a VH and a VL.

In one aspect, disclosed herein is a vector comprising a polynucleotide sequence encoding a heavy chain variable region (VH) of an anti-IL-8 antibody comprising a complementarity determining regions (HCDR) of said VH as set forth in Table 1F, and a light chain variable region (VL) of an anti-IL-8 antibody comprising a complementarity determining regions (LCDR) of said VL as set forth in Table 1F.

In one aspect, disclosed herein is a host cell comprising the vector comprising a polynucleotide sequence encoding a heavy chain variable region (VH) of an anti-IL-8 antibody comprising a complementarity determining regions (HCDR) of said VH as set forth in Table 1F, and a light chain variable region (VL) of an anti-IL-8 antibody comprising a complementarity determining regions (LCDR) of said VL as set forth in Table 1F.

In one aspect, disclosed herein is a method of producing an anti-IL-8 antibody having complementarity determining region (CDR) sequences as set forth in Table 1F, said method comprising the step of culturing the host cell of claim 25 under conditions conducive to expressing said vector in said host cell, and expressing said polynucleotide sequences comprised in said vector, thereby producing an anti-IL-8 antibody having complementarity determining region (CDR) sequences as set forth in Table 1F.

In one aspect, disclosed herein is a method of inhibiting tumor or cancer formation or growth or a combination thereof in a subject in need, said method comprising the step of administering to said subject an anti-IL-8 antibody comprising an antibody antigen-binding domain comprising a heavy chain variable region (VH) and a light chain variable region (VL), wherein the amino acid sequences of a VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35, thereby inhibiting tumor formation or growth or a combination thereof in said subject.

In a related aspect, a method of inhibiting, inhibits neutrophil or monocyte activation, or a combination thereof within a tumor microenvironment. In a further related aspect, inhibiting reduces activation of neutrophils or monocytes, or a combination thereof, within a tumor microenvironment. In another further related aspect, inhibiting decreases viability of pre-cancerous stem cells or tumor cells. In another related aspect, pre-cancerous stem cells comprise pre-leukemia stem cells. In yet another related aspect, a cancer or tumor comprises a hematological cancer. In a further related aspect, a hematological cancer comprises leukemia, lymphoma, myeloma, acute myeloid leukemia (AML), acute promyelocytic leukemia, erythroleukemia, biphenotypic B myelomonocytic leukemia, or myelodysplastic syndromes (MDS). In yet another related aspect, a cancer or tumor comprises a solid cancer or tumor. In a further related aspect, a solid cancer or tumor comprises sarcoma, osteosarcoma, squamous cell carcinoma of the head and neck, non-small-cell lung carcinoma, bladder cancer, pancreatic cancer, or pancreatic ductal adenocarcinoma.

In one aspect, disclosed herein is a method of inhibiting tumor or cancer formation or growth or a combination thereof in a subject in need, said method comprising the step of administering to said subject an anti-IL-8 antibody having complementarity determining region (CDR) sequences as set forth in Table 1F, wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, wherein said HCDR1, HCDR2, HCDR3, LCDR1, LCDR2 and LCDR3 for each of said antibody comprise the amino acid sequences as set forth in Table 1F, thereby inhibiting tumor formation or growth or a combination thereof in said subject.

In a related aspect, inhibiting inhibits neutrophil or monocyte activation, or a combination thereof within a tumor microenvironment. In another related aspect, inhibiting inhibits activation of neutrophils or monocytes, or a combination thereof, within a tumor microenvironment. In a further related aspect, inhibiting decreases viability of pre-cancerous stem cells or tumor cells. In yet another related aspect, pre-cancerous stem cells comprise pre-leukemic stem cells. In still another related aspect, a cancer or tumor comprises a hematological cancer. In a further related aspect, a hematological cancer comprises leukemia, lymphoma, myeloma, acute myeloid leukemia (AML), acute promyelocytic leukemia, erythroleukemia, biphenotypic B myelomonocytic leukemia, or myelodysplastic syndromes (MDS). In a further related aspect, a cancer or tumor comprises a solid cancer or tumor. In a further related aspect, a solid cancer or tumor comprises sarcoma, osteosarcoma, squamous cell carcinoma of the head and neck, non-small-cell lung carcinoma, bladder cancer, pancreatic cancer, or pancreatic ductal adenocarcinoma.

In another related aspect to a method of inhibiting tumor or cancer formation or growth, a subject is a human.

In one aspect, disclosed herein is a method of treating a subject suffering from a disease, said method comprising the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35, thereby treating said disease in said subject.

In a related aspect, a cancer or tumor comprises a hematological cancer. In a further related aspect, a hematological cancer comprises leukemia, lymphoma, myeloma, acute myeloid leukemia (AML), acute promyelocytic leukemia, erythroleukemia, biphenotypic B myelomonocytic leukemia, or myelodysplastic syndromes (MDS). In another related aspect, a cancer or tumor comprises a solid cancer or tumor. In a further related aspect, a solid cancer or tumor comprises sarcoma, osteosarcoma, squamous cell carcinoma of the head and neck, non-small-cell lung carcinoma, bladder cancer, pancreatic cancer, or pancreatic ductal adenocarcinoma.

In one aspect, disclosed herein is a method of treating a subject suffering from a disease, said method comprising the step of administering to said subject an anti-IL-8 antibody having complementarity determining region (CDR) sequences as set forth in Table 1F, wherein the disease comprises a cancer or tumor or a viral infection or a disease associated with inflammation or a combination thereof, and wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, wherein said HCDR1, HCDR2, HCDR3, LCDR1, LCDR2 and LCDR3 for each of said antibody comprise the amino acid sequences as set forth in Table 1F, thereby treating said disease in said subject.

In a related aspect, a cancer or tumor comprises a hematological cancer. In a further related aspect, a hematological cancer comprises leukemia, lymphoma, myeloma, acute myeloid leukemia (AML), acute promyelocytic leukemia, erythroleukemia, biphenotypic B myelomonocytic leukemia, or myelodysplastic syndromes (MDS). In another related aspect, a cancer or tumor comprises a solid cancer or tumor. In a further related aspect, a solid cancer or tumor comprises sarcoma, osteosarcoma, squamous cell carcinoma of the head and neck, non-small-cell lung carcinoma, bladder cancer, pancreatic cancer, or pancreatic ductal adenocarcinoma.

In a related aspect to a method of treating a subject suffering from a disease, a subject is a human.

In one aspect, disclosed herein is a method of inhibiting tumor or cancer formation or growth or a combination thereof in a subject in need, said method comprising the step of administering to said subject a polynucleotide encoding an anti-IL-8 antibody comprising an antibody antigen-binding domain comprising a heavy chain variable region (VH) and a light chain variable region (VL), wherein the polynucleotide sequences encoding the VH-VL pair are selected from the paired sequences set forth in SEQ ID NOs: 113 and 114, SEQ ID NOs: 115 and 116, SEQ ID NOs: 117 and 118, SEQ ID NOs: 119 and 120, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39, thereby inhibiting tumor formation or growth or a combination thereof in said subject. In a related aspect, inhibiting inhibits neutrophil or monocyte activation, or a combination thereof within a tumor microenvironment. In another related aspect, inhibiting reduces activation of neutrophils or monocytes, or a combination thereof, within a tumor microenvironment. In yet another related aspect, inhibiting decreases viability of pre-cancerous stem cells or tumor cells. In another related aspect, pre-cancerous stem cells comprise pre-leukemia stem cells. In a further related aspect, a cancer or tumor comprises a hematological cancer. In still a further related aspect, a hematological cancer comprises leukemia, lymphoma, myeloma, acute myeloid leukemia (AML), acute promyelocytic leukemia, erythroleukemia, biphenotypic B myelomonocytic leukemia, or myelodysplastic syndromes (MDS). In a further related aspect, a cancer or tumor comprises a solid cancer or tumor. In still a further related aspect, a solid cancer or tumor comprises sarcoma, osteosarcoma, squamous cell carcinoma of the head and neck, non-small-cell lung carcinoma, bladder cancer, pancreatic cancer, or pancreatic ductal adenocarcinoma.

In one aspect, disclosed herein is a method of treating a subject suffering from a disease comprising the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the polynucleotide sequences encoding the VH-VL pair are selected from the paired sequences set forth in SEQ ID NOs: 113 and 114, SEQ ID NOs: 115 and 116, SEQ ID NOs: 117 and 118, SEQ ID NOs: 119 and 120, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39, thereby treating said disease in said subject.

In a related aspect for a method of treating a subject suffering from a disease, a cancer or tumor comprises a hematological cancer. In a further related aspect, a hematological cancer comprises leukemia, lymphoma, myeloma, acute myeloid leukemia (AML), acute promyelocytic leukemia, erythroleukemia, biphenotypic B myelomonocytic leukemia, or myelodysplastic syndromes (MDS). In another related aspect for a method of treating a subject suffering from a disease, a cancer or tumor comprises a solid cancer or tumor. In a further related aspect, a solid cancer or tumor comprises sarcoma, osteosarcoma, squamous cell carcinoma of the head and neck, non-small-cell lung carcinoma, bladder cancer, pancreatic cancer, or pancreatic ductal adenocarcinoma.

In another related aspect of methods disclosed herein, a subject is a human.

### BRIEF DESCRIPTION OF THE DRAWINGS

The subject matter regarded as the invention is particularly pointed out and distinctly claimed in the concluding portion of the specification. The invention, however, both as to organization and method of operation, together with objects, features, and advantages thereof, may best be understood by reference to the following detailed description when read with the accompanying drawings in which:

FIG. 1: Expression analysis of mAbs. Expression of antibody molecules was studied under non-reducing and reducing conditions.

FIG. 2: Size-exclusion analysis of mAbs. Size-exclusion analysis was conducted in HPLC.

FIG. 3: Binding of mAbs to human IL-8 by ELISA. Binding kinetics of mAbs to human IL-8 was measured by cell-free ELISA.

FIG. 4: Blocking IL-8 binding to its cell surface receptor, CXCR2. Blocking of binding of IL-8 to CXCR2 receptor by mAbs was measured in cell-based assay by flow cytometry.

FIG. 5: Inhibition of IL-8-induced NFκB activity by mAbs. Blocking of IL-8-induced NFκB activity by mAbs was measured in HEK293-CXCR1 cell-based reporter assay.

FIG. 6: Cell surface expression of CXCR1 and CXCR2 in cell lines by IHC. Expression of CXCR1 and CXCR2 receptors was detected at varying levels in osteosarcoma and pancreatic tumor cell lines by IHC. The micrograph images show osteosarcoma cells lines (143B, SaOS-2, OS17) and a pancreatic cancer cell line (PANC-1) expressed both CXCR2 and CXCR1 receptors.

FIG. 7: Cell line IL-8 secretion by ELISA. Secretion of IL-8 was monitored by ELISA in the supernatants of MG-63, U2-OS, SAOS-2 and 143-B osteosarcoma cells.

FIG. 8: Inhibition of IL-8-induced CXCR2 internalization by mAbs. Internalization of CXCR2 receptor was monitored by flow cytometry. In THP-1 cells, CXCR2 receptor internalization was induced by IL-8 treatment, while CXCR2 internalization was blocked in a dose-dependent manner by cotreatment with mAbs.

FIG. 9: Inhibition of IL8 signaling in human neutrophils by STLX18 antibody. STLX18 monoclonal antibody inhibited IL8-induced downstream signaling in human neutrophils, as monitored by Western Blot using phospho-specific antibodies against ERK and AKT. Actin was used as a loading control.

FIG. 10: Neutrophil migration towards IL8 is inhibited by STLX18 antibody. Human neutrophil migration (towards IL8 source) across a porous membrane is inhibited by the STLX18 antibody.

FIGS. 11A and 11B: Monosodium Urate (MSU)-induced knee swelling inhibited by STLX18 antibody. Treatment with STLX18 antibody reduced the swelling of MSU injected joints (FIG. 11B) while control joints infused with saline did not change in size (FIG. 11A) and STLX18 showed no effect on control knee diameter.

### DETAILED DESCRIPTION

In the following detailed description, numerous specific details are set forth in order to provide a thorough understanding of the anti-IL-8 monoclonal antibodies (mAb) described and exemplified herein, and therapeutic uses thereof. However, it will be understood by those skilled in the art that production and use of the IL-8 binding anti-IL-8 may in certain cases, be practiced without these specific details. In other instances, well-known methods, procedures, and components have not been described in detail so as not to obscure the disclosure presented herein.

IL-8 is a potent proinflammatory cytokine, secreted by various cells, primarily macrophages. It binds the seven transmembrane GPCRs, CXCR1 and CXCR2, on cell-surface of neutrophils and monocytes and impacts their activation and migration to infected areas. A growing body of evidence suggests a role of IL-8 signaling in cancer, in both hematological malignancies and solid tumors.

Disclosed herein are humanized IL-8 binding mAbs or fragments thereof. In some embodiments, these anti-IL-8 mAb or fragments thereof may be used in a therapeutic method for inhibiting tumor formation or tumor growth, or a combination thereof. In some embodiments, these anti-IL-8 mAb or fragments thereof may be used in a therapeutic method for treating a subject suffering from a disease such as a cancer or a tumor, or a viral infection.

Throughout this application, various references or publications are cited. Disclosures of these references or publications in their entireties are hereby incorporated by reference into this application in order to more fully describe the state of the art to which this invention pertains.

As used herein, the term “antibody” may be used interchangeably with the terms “immunoglobulin” or “IgG”, having all the same qualities and meanings. An antibody binding domain or an antigen binding site can be a fragment of an antibody or a genetically engineered product of one or more fragments of the antibody, which fragment is involved in specifically binding with a target antigen. By “specifically binding” is meant that the binding is selective for the antigen of interest and can be discriminated from unwanted or nonspecific interactions. For example, an antibody is said to specifically bind an IL-8 epitope when the equilibrium dissociation constant is ≤10⁻⁵, 10⁻⁶, or 10⁻⁷ M. In some embodiments, the equilibrium dissociation constant may be ≤10⁻⁸ M or 10⁻⁹ M. In some further embodiments, the equilibrium dissociation constant may be ≤10⁻¹⁰ M, 10⁻¹¹ M, or 10⁻¹²M. In some embodiments, the equilibrium dissociation constant may be in the range of ≤10⁻⁵ M to 10⁻¹² M.

As used herein, the term “antibody” encompasses an antibody fragment or fragments that retain binding specificity including, but not limited to, IgG, heavy chain variable region (VH), light chain variable region (VL), Fab fragments, F(ab′)2 fragments, scFv fragments, Fv fragments, a multi-specific antibody, a bi-specific antibody, a tri-specific antibody, a single chain antibody, a heavy chain antibody, a nanobody, minibodies, diabodies, triabodies, tetrabodies, and single domain antibodies (see, e.g., Hudson and Souriau, Nature Med. 9: 129-134 (2003)). Also encompassed are humanized, primatized, and chimeric antibodies as these terms are generally understood in the art. In certain embodiments, antibodies disclosed herein are engineered, in other words non-naturally produced antibodies, designed to bind IL-8 and provide certain functional activities.

In certain embodiments, the antibody comprises a heavy chain constant region, such as an IgG1, IgG2, IgG3, IgG4, IgA, IgE, IgM or IgD constant region. In some embodiments, the heavy chain constant region is an IgG1 heavy chain constant region. In some embodiments, the antibody comprises a light chain constant region, either a kappa light chain constant region or a lambda light chain constant region. In some embodiments, an antibody lacks a constant region, for example, a Fab fragment or a single chain Fv fragment.

As used herein, the term “heavy chain variable region” may be used interchangeably with the term “VH domain” or VH region” or the term “VH”, having all the same meanings and qualities. As used herein, the term “light chain variable region” may be used interchangeably with the term “VL domain” or “VL region” or the term “VL”, having all the same meanings and qualities.

A skilled artisan would recognize that a “heavy chain variable region” or “VH” with regard to an antibody encompasses the fragment of the heavy chain that contains three complementarity determining regions (CDRs) interposed between flanking stretches known as framework regions. The framework regions are more highly conserved than the CDRs, and form a scaffold to support the CDRs. Similarly, a skilled artisan would also recognize that a “light chain variable region” or “VL” with regard to an antibody encompasses the fragment of the light chain that contains three CDRs interposed between framework regions.

As used herein, the term “complementarity determining region” or “CDR” refers to the hypervariable region(s) of a heavy or light chain variable region. Proceeding from the N-terminus, each of a heavy or light chain polypeptide has three CDRs denoted as “CDR1,” “CDR2,” and “CDR3”. Crystallographic analysis of a number of antigen-antibody complexes has demonstrated that the amino acid residues of CDRs form extensive contact with a bound antigen, wherein the most extensive antigen contact is with the heavy chain CDR3. Thus, the CDR regions are primarily responsible for the specificity of an antigen-binding site. In one embodiment, an antigen-binding site includes six CDRs, comprising the CDRs from each of a heavy and a light chain variable region. Examples of sets of six CDRs comprised within an IL-8 antigen-binding site are presented in Table 1F (See, Example 2).

As used herein, the term “framework region” or “FR” refers to the four flanking amino acid sequences which frame the CDRs of a heavy or light chain variable region. Some FR residues may contact bound antigen; however, FR residues are primarily responsible for folding the variable region into the antigen-binding site. In some embodiments, the FR residues responsible for folding the variable regions comprise residues directly adjacent to the CDRs. Within FRs, certain amino residues and certain structural features are very highly conserved. In this regard, all variable region sequences contain an internal disulfide loop of around 90 amino acid residues. When a variable region folds into an antigen binding site, the CDRs are displayed as projecting loop motifs that form an antigen-binding surface. It is generally recognized that there are conserved structural regions of FR that influence the folded shape of the CDR loops into certain “canonical” structures regardless of the precise CDR amino acid sequence. Furthermore, certain FR residues are known to participate in non-covalent interdomain contacts which stabilize the interaction of the antibody heavy and light chains.

Wu and Kabat (Tai Te Wu, Elvin A. Kabat. An analysis of the sequences of the variable regions of bence jones proteins and myeloma light chains and their implications for antibody complementarity. Journal of Experimental Medicine, 132, 2, 8 (1970); Kabat E A, Wu T T, Bilofsky H, Reid-Miller M, Perry H. Sequence of proteins of immunological interest. Bethesda: National Institute of Health; 1983. 323 (1983)) pioneered the alignment of antibody peptide sequences, and their contributions in this regard were several-fold: Firstly, through study of sequence similarities between variable domains, they identified correspondent residues that to a greater or lesser extent were homologous across all antibodies in all vertebrate species, inasmuch as they adopted similar three-dimensional structure, played similar functional roles, interacted similarly with neighboring residues, and existed in similar chemical environments. Secondly, they devised a peptide sequence numbering system in which homologous immunoglobulin residues were assigned the same position number. One skilled in the art can unambiguously assign to any variable domain sequence what is now commonly called Kabat numbering without reliance on any experimental data beyond the sequence itself. Thirdly, Kabat and Wu calculated variability for each Kabat-numbered sequence position, by which is meant the finding of few or many possible amino acids when variable domain sequences are aligned. They identified three contiguous regions of high variability embedded within four less variable contiguous regions. Kabat and Wu formally demarcated residues constituting these variable tracts, and designated these “complementarity determining regions” (CDRs), referring to chemical complementarity between antibody and antigen. A role in three-dimensional folding of the variable domain, but not in antigen recognition, was ascribed to the remaining less-variable regions, which are now termed “framework regions”. Fourth, Kabat and Wu established a public database of antibody peptide and nucleic acid sequences, which continues to be maintained and is well known to those skilled in the art.

Chothia and coworkers (Cyrus Chothia, Arthur M. Lesk. Canonical structures for the hypervariable regions of immunoglobulins. Journal of Molecular Biology, 196, 4, 8 (1987)) found that certain sub portions within Kabat CDRs adopt nearly identical peptide backbone conformations, despite having great diversity at the level of amino acid sequence. These sub portions were designated as L1, L2 and L3 or H1, H2 and H3, where the “L” and the “H” designates the light chain and the heavy chains regions, respectively. These regions may be referred to as Chothia CDRs, which have boundaries that overlap with Kabat CDRs.

More recent studies have shown that virtually all antibody binding residues fall within regions of structural consensus. (Kunik, V. et al., PloS Computational Biology 8(2):e1002388 (February 2012)). In some embodiments, these regions are referred to as antibody binding regions. It was shown that these regions can be identified from the antibody sequence as well.

IMGT® is the international ImMunoGeneTics information System®, (See, Nucleic Acids Res. 2015 January; 43 (Database issue):D413-22. doi: 10.1093/nar/gkui056. Epub 2014 Nov. 5 Free article. PMID: 25378316 LIGM:441 and Dev Comp Immunol. 2003 January; 27(1):55-77). IMGT is a unique numbering system for immunoglobulin and T cell receptor variable domains and Ig superfamily V-like domains (Lefranc et al., Dev Comp Immunol. 27: 55-77 (2003)). IMGT® presents a uniform numbering system for these IG and TcR variable domain sequences, based on aligning 5 or more IG and TcR variable region sequences, taking into account and combining the Kabat definition of FRs and CDRs, structural data, and Chothia's characterization of the hypervariable loops. IMGT is considered well known in the art as a universal numbering scheme for antibodies.

In some embodiments, identification of CDR regions uses the IMGT system of analysis. In some embodiments, identification of CDR regions uses a system of analysis based on Kabat.

Antigen binding sequences are conventionally located within the heavy chain and light chain variable regions of an antibody. An antibody may exist in various forms or having various domains including, without limitation, a complementarity determining region (CDR), a variable region (Fv), a VH domain, a VL domain, a single chain variable region (scFv), and a Fab fragment.

A person of ordinary skill in the art would appreciate that a scFv is a fusion polypeptide comprising the variable heavy chain (VH) and variable light chain (VL) regions of an immunoglobulin, connected by a short linker peptide, the linker may have, for example, 10 to about 25 amino acids.

A skilled artisan would also appreciate that the term “Fab” with regard to an antibody generally encompasses that portion of the antibody consisting of a single light chain (both variable and constant regions) bound to the variable region and first constant region of a single heavy chain by a disulfide bond, whereas F(ab′)2 comprises a fragment of a heavy chain comprising a VH domain and a light chain comprising a VL domain.

In some embodiments, an antibody encompasses whole antibody molecules, including monoclonal (mAb) and polyclonal antibodies. In some embodiments, an antibody encompasses an antibody fragment or fragments that retain binding specificity including, but not limited to, variable heavy chain (VH) fragments, variable light chain (VL) fragments, Fab fragments, F(ab′)2 fragments, scFv fragments, Fv fragments, minibodies, diabodies, triabodies, and tetrabodies.

### Anti-IL-8 Antibodies

In some embodiments, described herein is an isolated anti-IL-8 antibody comprising an antibody antigen-binding domain comprising a heavy chain variable region (VH) and a light chain variable region (VL), wherein the amino acid sequences of a VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35. In some embodiments, the amino acid sequence of a VH-VL pair is set forth in SEQ ID NOs: 2 and 3. In some embodiments, the amino acid sequence of a VH-VL pair is set forth in SEQ ID NOs: 4 and 5. In some embodiments, the amino acid sequence of a VH-VL pair is set forth in SEQ ID NOs: 6 and 7. In some embodiments, the amino acid sequence of a VH-VL pair is set forth in SEQ ID NOs: 8 and 9. In some embodiments, the amino acid sequence of a VH-VL pair is set forth in SEQ ID NOs: 10 and 11. In some embodiments, the amino acid sequence of a VH-VL pair is set forth in SEQ ID NOs: 12 and 13. In some embodiments, the amino acid sequence of a VH-VL pair is set forth in SEQ ID NOs: 14 and 15. In some embodiments, the amino acid sequence of a VH-VL pair is set forth in SEQ ID NOs: 16 and 17. In some embodiments, the amino acid sequence of a VH-VL pair is set forth in SEQ ID NOs: 18 and 19. In some embodiments, the amino acid sequence of a VH-VL pair is set forth in SEQ ID NOs: 20 and 21. In some embodiments, the amino acid sequence of a VH-VL pair is set forth in SEQ ID NOs: 22 and 23. In some embodiments, the amino acid sequence of a VH-VL pair is set forth in SEQ ID NOs: 24 and 25. In some embodiments, the amino acid sequence of a VH-VL pair is set forth in SEQ ID NOs: 26 and 27. In some embodiments, the amino acid sequence of a VH-VL pair is set forth in SEQ ID NOs: 28 and 29. In some embodiments, the amino acid sequence of a VH-VL pair is set forth in SEQ ID NOs: 30 and 31. In some embodiments, the amino acid sequence of a VH-VL pair is set forth in SEQ ID NOs: 32 and 33. In some embodiments, the amino acid sequence of a VH-VL pair is set forth in SEQ ID NOs: 34 and 35.

In some embodiments, the amino acid sequence of a VH, and or a VL, is selected from a homolog having at least 80% identity of any of the VH or VL sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35. In some embodiments, the amino acid sequence of a VH, or a VL, is selected from a homolog having at least 80% identity of the VH and or VL sequences set forth in SEQ ID NOs: 2 and 3. In some embodiments, the amino acid sequence of a VH, or a VL, is selected from a homolog having at least 80% identity of the VH and or VL sequences set forth in SEQ ID NOs: 4 and 5. In some embodiments, the amino acid sequence of a VH, or a VL, is selected from a homolog having at least 80% identity of the VH and or VL sequences set forth in SEQ ID NOs: 6 and 7. In some embodiments, the amino acid sequence of a VH, or a VL, is selected from a homolog having at least 80% identity of the VH and or VL sequences set forth in SEQ ID NOs: 8 and 9. In some embodiments, the amino acid sequence of a VH, or a VL, is selected from a homolog having at least 80% identity of the VH and or VL sequences set forth in SEQ ID NOs: 10 and 11. In some embodiments, the amino acid sequence of a VH, or a VL, is selected from a homolog having at least 80% identity of the VH and or VL sequences set forth in SEQ ID NOs: 12 and 13. In some embodiments, the amino acid sequence of a VH, or a VL, is selected from a homolog having at least 80% identity of the VH and or VL sequences set forth in SEQ ID NOs: 14 and 15. In some embodiments, the amino acid sequence of a VH, or a VL, is selected from a homolog having at least 80% identity of the VH and or VL sequences set forth in SEQ ID NOs: 16 and 17. In some embodiments, the amino acid sequence of a VH, or a VL, is selected from a homolog having at least 80% identity of the VH and or VL sequences set forth in SEQ ID NOs: 18 and 19. In some embodiments, the amino acid sequence of a VH, or a VL, is selected from a homolog having at least 80% identity of the VH and or VL sequences set forth in SEQ ID NOs: 20 and 21. In some embodiments, the amino acid sequence of a VH, or a VL, is selected from a homolog having at least 80% identity of the VH and or VL sequences set forth in SEQ ID NOs: 22 and 23. In some embodiments, the amino acid sequence of a VH, or a VL, is selected from a homolog having at least 80% identity of the VH and or VL sequences set forth in SEQ ID NOs: 24 and 25. In some embodiments, the amino acid sequence of a VH, or a VL, is selected from a homolog having at least 80% identity of the VH and or VL sequences set forth in SEQ ID NOs: 26 and 27. In some embodiments, the amino acid sequence of a VH, or a VL, is selected from a homolog having at least 80% identity of the VH and or VL sequences set forth in SEQ ID NOs: 28 and 29. In some embodiments, the amino acid sequence of a VH, or a VL, is selected from a homolog having at least 80% identity of the VH and or VL sequences set forth in SEQ ID NOs: 30 and 31. In some embodiments, the amino acid sequence of a VH, or a VL, is selected from a homolog having at least 80% identity of the VH and or VL sequences set forth in SEQ ID NOs: 32 and 33. In some embodiments, the amino acid sequence of a VH, or a VL, is selected from a homolog having at least 80% identity of the VH and or VL sequences set forth in SEQ ID NOs: 34 and 35.

In some embodiments, the homolog has 100% sequence identity within the three VH CDR (HCDR1, HCDR2, HCDR3). In some embodiments, the homolog has 100% sequence identity within the three VL LCDR1, LCDR2, and LCDR3. In some embodiments, the amino acid sequence of a VH is selected from a homolog having at least 80% identity of any of the sequences set forth in SEQ ID NOS: 2, 4, 6, 8, 10, 12, 14, 16, 18, 20, 22, 24, 26, 28, 30, 32, and 34. In some embodiments, the amino acid sequence of a VH comprises a homolog having at least 80% identity with the sequence set forth in SEQ ID NO: 2. In some embodiments, the amino acid sequence of a VH comprises a homolog having at least 80% identity with the sequence set forth in SEQ ID NO: 4. In some embodiments, the amino acid sequence of a VH comprises a homolog having at least 80% identity with the sequence set forth in SEQ ID NO: 6. In some embodiments, the amino acid sequence of a VH comprises a homolog having at least 80% identity with the sequence set forth in SEQ ID NO: 8. In some embodiments, the amino acid sequence of a VH comprises a homolog having at least 80% identity with the sequence set forth in SEQ ID NO: 10. In some embodiments, the amino acid sequence of a VH comprises a homolog having at least 80% identity with the sequence set forth in SEQ ID NO: 12. In some embodiments, the amino acid sequence of a VH comprises a homolog having at least 80% identity with the sequence set forth in SEQ ID NO: 14. In some embodiments, the amino acid sequence of a VH comprises a homolog having at least 80% identity with the sequence set forth in SEQ ID NO: 16. In some embodiments, the amino acid sequence of a VH comprises a homolog having at least 80% identity with the sequence set forth in SEQ ID NO: 18. In some embodiments, the amino acid sequence of a VH comprises a homolog having at least 80% identity with the sequence set forth in SEQ ID NO: 20. In some embodiments, the amino acid sequence of a VH comprises a homolog having at least 80% identity with the sequence set forth in SEQ ID NO: 22. In some embodiments, the amino acid sequence of a VH comprises a homolog having at least 80% identity with the sequence set forth in SEQ ID NO: 24. In some embodiments, the amino acid sequence of a VH comprises a homolog having at least 80% identity with the sequence set forth in SEQ ID NO: 26. In some embodiments, the amino acid sequence of a VH comprises a homolog having at least 80% identity with the sequence set forth in SEQ ID NO: 28. In some embodiments, the amino acid sequence of a VH comprises a homolog having at least 80% identity with the sequence set forth in SEQ ID NO: 30. In some embodiments, the amino acid sequence of a VH comprises a homolog having at least 80% identity with the sequence set forth in SEQ ID NO: 32 In some embodiments, the amino acid sequence of a VH comprises a homolog having at least 80% identity with the sequence set forth in SEQ ID NO: 34. In some embodiments, the amino acid sequence of a VL is selected from a homolog having at least 80% identity of any of the sequences set forth in SEQ ID NOS: 3, 5, 7, 9, 11, 13, 15, 17, 19, 21, 23, 25 27, 29, 31, 33, and 35. In some embodiments, the amino acid sequence of a VL comprises a homolog having at least 80% identity with the sequence set forth in SEQ ID NO: 3. In some embodiments, the amino acid sequence of a VL comprises a homolog having at least 80% identity with the sequence set forth in SEQ ID NO: 5. In some embodiments, the amino acid sequence of a VL comprises a homolog having at least 80% identity with the sequence set forth in SEQ ID NO: 7. In some embodiments, the amino acid sequence of a VL comprises a homolog having at least 80% identity with the sequence set forth in SEQ ID NO: 9. In some embodiments, the amino acid sequence of a VL comprises a homolog having at least 80% identity with the sequence set forth in SEQ ID NO: 11. In some embodiments, the amino acid sequence of a VL comprises a homolog having at least 80% identity with the sequence set forth in SEQ ID NO: 13. In some embodiments, the amino acid sequence of a VL comprises a homolog having at least 80% identity with the sequence set forth in SEQ ID NO: 15. In some embodiments, the amino acid sequence of a VL comprises a homolog having at least 80% identity with the sequence set forth in SEQ ID NO: 17. In some embodiments, the amino acid sequence of a VL comprises a homolog having at least 80% identity with the sequence set forth in SEQ ID NO: 19. In some embodiments, the amino acid sequence of a VL comprises a homolog having at least 80% identity with the sequence set forth in SEQ ID NO: 21. In some embodiments, the amino acid sequence of a VL comprises a homolog having at least 80% identity with the sequence set forth in SEQ ID NO: 23. In some embodiments, the amino acid sequence of a VL comprises a homolog having at least 80% identity with the sequence set forth in SEQ ID NO: 25. In some embodiments, the amino acid sequence of a VL comprises a homolog having at least 80% identity with the sequence set forth in SEQ ID NO: 27. In some embodiments, the amino acid sequence of a VL comprises a homolog having at least 80% identity with the sequence set forth in SEQ ID NO: 29. In some embodiments, the amino acid sequence of a VL comprises a homolog having at least 80% identity with the sequence set forth in SEQ ID NO: 31. In some embodiments, the amino acid sequence of a VL comprises a homolog having at least 80% identity with the sequence set forth in SEQ ID NO: 33. In some embodiments, the amino acid sequence of a VL comprises a homolog having at least 80% identity with the sequence set forth in SEQ ID NO: 35. The variable chain regions (VH or VL) and the respective CDRs within each of these VH or VL regions may be determined based on Tables 1A-1F as provided in Example 2.

In some embodiments, a VH homolog has at least 85% identity, at least 87%, at least 89%, at least 91%, at least 93%, at least 95%, at least 96%, at least 97%, at least 98%, or at least 99% identity to a VH polypeptide described herein, as determined using BlastP software of the National Center of Biotechnology Information (NCBI) using default parameters. In some embodiments, a VL homolog has at least 85% identity, at least 87%, at least 89%, at least 91%, at least 93%, at least 95%, at least 96%, at least 97%, at least 98%, or at least 99% identity to a VL polypeptide described herein, as determined using BlastP software of the National Center of Biotechnology Information (NCBI) using default parameters. In some embodiments, a VH homolog has at least 85% identity, at least 87%, at least 89%, at least 91%, at least 93%, at least 95%, at least 96%, at least 97%, at least 98%, or at least 99% identity to a VH polypeptide described herein, and a VL homolog has at least 85% identity, at least 87%, at least 89%, at least 91%, at least 93%, at least 95%, at least 96%, at least 97%, at least 98%, or at least 99% identity to a VL polypeptide described herein, as determined using BlastP software of the National Center of Biotechnology Information (NCBI) using default parameters.

A skilled artisan would appreciate that the term “homology”, and grammatical forms thereof, encompasses the degree of similarity between two or more structures. The term “homologous sequences” refers to regions in macromolecules that have a similar order of monomers.

When used in relation to polypeptide (or protein) sequences, the term “homology” refers to the degree of similarity between two or more polypeptide (or protein) sequences (e.g., genes) or fragments thereof. Typically, the degree of similarity between two or more polypeptide (or protein) sequences refers to the degree of similarity of the composition, order, or arrangement of two or more amino acid of the two or more polypeptides (or proteins). The two or more polypeptides (or proteins) may be of the same or different species or group. The term “percent homology” when used in relation to polypeptide (or protein) sequences, refers generally to a percent degree of similarity between the amino acid sequences of two or more polypeptide (or protein) sequences. The term “homologous polypeptides” or “homologous proteins” generally refers to polypeptides or proteins, respectively, that have amino acid sequences and functions that are similar. Such homologous polypeptides or proteins may be related by having amino acid sequences and functions that are similar but are derived or evolved from different or the same species or may be synthesized using genetic engineering techniques well known to the skill artisan.

In some embodiments, described herein is an isolated anti-IL-8 antibody comprising an antibody antigen-binding domain comprising a heavy chain variable region (VH) and a light chain variable region (VL), wherein the heavy chain variable region comprises complementarity determining region 1 (HCDR1), HCDR2 and HCDR3, and the light chain variable region having complementarity determining region 1 (LCDR1), LCDR2 and LCDR3. In some embodiments, an isolated anti-IL-8 antibody comprises complementarity determining region (CDR) sequences as set forth in Table 1F, wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, wherein said HCDR1, HCDR2, HCDR3, LCDR1, LCDR2 and LCDR3 for each of said antibody comprise the amino acid sequences as set forth in Table 1F. In some embodiments, an isolated anti-IL-8 antibody comprises CDR sequences as set forth in Table 1F, wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, said HCDR1, HCDR2, HCDR3, LCDR1, LCDR2 and LCDR3 for said antibody comprising s the amino acid sequences as set forth:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109.

In some embodiments, an isolated anti-IL-8 antibody comprising CDR sequences comprises a heavy chain variable region having heavy chain complementarity determining region (HCDR) 1, HCDR2 and HCDR3 and a light chain variable region having light chain complementarity determining region (LCDR) 1, LCDR2 and LCDR3, said HCDR1, HCDR2, HCDR3, LCDR1, LCDR2 and LCDR3 for said antibody comprising the amino acid sequences as set forth in SEQ ID NO: 40, SEQ ID NO: 43, SEQ ID NO: 50, SEQ ID NO: 57, SEQ ID NO: 64, and SEQ ID NO: 73, respectively. In some embodiments, said HCDR1, HCDR2, HCDR3, LCDR1, LCDR2 and LCDR3 for said antibody comprising the amino acid sequences as set forth in SEQ ID NO: 40, SEQ ID NO: 43, SEQ ID NO: 51, SEQ ID NO: 58, SEQ ID NO: 65, and SEQ ID NO: 74, respectively. In some embodiments, said HCDR1, HCDR2, HCDR3, LCDR1, LCDR2 and LCDR3 for said antibody comprising the amino acid sequences as set forth in SEQ ID NO: 41, SEQ ID NO: 44, SEQ ID NO: 52, SEQ ID NO: 59, SEQ ID NO: 66, and SEQ ID NO: 75, respectively. In some embodiments, said HCDR1, HCDR2, HCDR3, LCDR1, LCDR2 and LCDR3 for said antibody comprising the amino acid sequences as set forth in SEQ ID NO: 41, SEQ ID NO: 44, SEQ ID NO: 53, SEQ ID NO: 60, SEQ ID NO: 67, and SEQ ID NO: 76, respectively. In some embodiments, said HCDR1, HCDR2, HCDR3, LCDR1, LCDR2 and LCDR3 for said antibody comprising the amino acid sequences as set forth in SEQ ID NO: 40, SEQ ID NO: 43, SEQ ID NO: 50, SEQ ID NO: 57, SEQ ID NO: 68, and SEQ ID NO: 73, respectively. In some embodiments, said HCDR1, HCDR2, HCDR3, LCDR1, LCDR2 and LCDR3 for said antibody comprising the amino acid sequences as set forth in SEQ ID NO: 40, SEQ ID NO: 43, SEQ ID NO: 50, SEQ ID NO: 57, SEQ ID NO: 69, and SEQ ID NO: 73, respectively. In some embodiments, said HCDR1, HCDR2, HCDR3, LCDR1, LCDR2 and LCDR3 for said antibody comprising the amino acid sequences as set forth in SEQ ID NO: 41, SEQ ID NO: 45, SEQ ID NO: 53, SEQ ID NO: 61, SEQ ID NO: 70, and SEQ ID NO: 77, respectively. In some embodiments, said HCDR1, HCDR2, HCDR3, LCDR1, LCDR2 and LCDR3 for said antibody comprising the amino acid sequences as set forth in SEQ ID NO: 40, SEQ ID NO: 46, SEQ ID NO: 54, SEQ ID NO: 62, SEQ ID NO: 71, and SEQ ID NO: 78, respectively. In some embodiments, said HCDR1, HCDR2, HCDR3, LCDR1, LCDR2 and LCDR3 for said antibody comprising the amino acid sequences as set forth in SEQ ID NO: 42, SEQ ID NO: 47, SEQ ID NO: 53, SEQ ID NO: 63, SEQ ID NO: 72, and SEQ ID NO: 79, respectively. In some embodiments, said HCDR1, HCDR2, HCDR3, LCDR1, LCDR2 and LCDR3 for said antibody comprising the amino acid sequences as set forth in SEQ ID NO: 41, SEQ ID NO: 48, SEQ ID NO: 53, SEQ ID NO: 60, SEQ ID NO: 67, and SEQ ID NO: 76, respectively. In some embodiments, said HCDR1, HCDR2, HCDR3, LCDR1, LCDR2 and LCDR3 for said antibody comprising the amino acid sequences as set forth in SEQ ID NO: 41, SEQ ID NO: 49, SEQ ID NO: 53, SEQ ID NO: 60, SEQ ID NO: 67, and SEQ ID NO: 76, respectively. In some embodiments, said HCDR1, HCDR2, HCDR3, LCDR1, LCDR2 and LCDR3 for said antibody comprising the amino acid sequences as set forth in SEQ ID NO: 41, SEQ ID NO: 44, SEQ ID NO: 55, SEQ ID NO: 60, SEQ ID NO: 67, and SEQ ID NO: 76, respectively. In some embodiments, said HCDR1, HCDR2, HCDR3, LCDR1, LCDR2 and LCDR3 for said antibody comprising the amino acid sequences as set forth in SEQ ID NO: 41, SEQ ID NO: 44, SEQ ID NO: 56, SEQ ID NO: 60, SEQ ID NO: 67, and SEQ ID NO: 76, respectively. In some embodiments, said HCDR1, HCDR2, HCDR3, LCDR1, LCDR2 and LCDR3 for said antibody comprising the amino acid sequences as set forth in SEQ ID NO: 41, SEQ ID NO: 48, SEQ ID NO: 55, SEQ ID NO: 60, SEQ ID NO: 67, and SEQ ID NO: 76, respectively. In some embodiments, said HCDR1, HCDR2, HCDR3, LCDR1, LCDR2 and LCDR3 for said antibody comprising the amino acid sequences as set forth in SEQ ID NO: 41, SEQ ID NO: 48, SEQ ID NO: 56, SEQ ID NO: 60, SEQ ID NO: 67, and SEQ ID NO: 76, respectively. In some embodiments, said HCDR1, HCDR2, HCDR3, LCDR1, LCDR2 and LCDR3 for said antibody comprising the amino acid sequences as set forth in SEQ ID NO: 41, SEQ ID NO: 49, SEQ ID NO: 55, SEQ ID NO: 60, SEQ ID NO: 67, and SEQ ID NO: 76, respectively. In some embodiments, said HCDR1, HCDR2, HCDR3, LCDR1, LCDR2 and LCDR3 for said antibody comprising the amino acid sequences as set forth in SEQ ID NO: 41, SEQ ID NO: 49, SEQ ID NO: 56, SEQ ID NO: 60, SEQ ID NO: 67, and SEQ ID NO: 76, respectively. In some embodiments, said HCDR1, HCDR2, HCDR3, LCDR1, LCDR2 and LCDR3 for said antibody comprising the amino acid sequences as set forth in SEQ ID NO: 80, SEQ ID NO: 83, SEQ ID NO: 50, SEQ ID NO: 90, SEQ ID NO: 97, and SEQ ID NO: 106, respectively. In some embodiments, said HCDR1, HCDR2, HCDR3, LCDR1, LCDR2 and LCDR3 for said antibody comprising the amino acid sequences as set forth in SEQ ID NO: 80, SEQ ID NO: 83, SEQ ID NO: 51, SEQ ID NO: 91, SEQ ID NO: 98, and SEQ ID NO: 107, respectively. In some embodiments, said HCDR1, HCDR2, HCDR3, LCDR1, LCDR2 and LCDR3 for said antibody comprising the amino acid sequences as set forth in SEQ ID NO: 81, SEQ ID NO: 84, SEQ ID NO: 52, SEQ ID NO: 92, SEQ ID NO: 99, and SEQ ID NO: 108, respectively. In some embodiments, said HCDR1, HCDR2, HCDR3, LCDR1, LCDR2 and LCDR3 for said antibody comprising the amino acid sequences as set forth in SEQ ID NO: 81, SEQ ID NO: 84, SEQ ID NO: 53, SEQ ID NO: 93, SEQ ID NO: 100, and SEQ ID NO: 109, respectively. In some embodiments, said HCDR1, HCDR2, HCDR3, LCDR1, LCDR2 and LCDR3 for said antibody comprising the amino acid sequences as set forth in SEQ ID NO: 80, SEQ ID NO: 83, SEQ ID NO: 50, SEQ ID NO: 90, SEQ ID NO: 101, and SEQ ID NO: 106, respectively. In some embodiments, said HCDR1, HCDR2, HCDR3, LCDR1, LCDR2 and LCDR3 for said antibody comprising the amino acid sequences as set forth in SEQ ID NO: 80, SEQ ID NO: 83, SEQ ID NO: 50, SEQ ID NO: 90, SEQ ID NO: 102, and SEQ ID NO: 106, respectively. In some embodiments, said HCDR1, HCDR2, HCDR3, LCDR1, LCDR2 and LCDR3 for said antibody comprising the amino acid sequences as set forth in SEQ ID NO: 81, SEQ ID NO: 85, SEQ ID NO: 53, SEQ ID NO: 94, SEQ ID NO: 103, and SEQ ID NO: 110, respectively. In some embodiments, said HCDR1, HCDR2, HCDR3, LCDR1, LCDR2 and LCDR3 for said antibody comprising the amino acid sequences as set forth in SEQ ID NO: 80, SEQ ID NO: 86, SEQ ID NO: 54, SEQ ID NO: 95, SEQ ID NO: 104, and SEQ ID NO: 111, respectively. In some embodiments, said HCDR1, HCDR2, HCDR3, LCDR1, LCDR2 and LCDR3 for said antibody comprising the amino acid sequences as set forth in SEQ ID NO: 82, SEQ ID NO: 87, SEQ ID NO: 53, SEQ ID NO: 93, SEQ ID NO: 105, and SEQ ID NO: 112, respectively. In some embodiments, said HCDR1, HCDR2, HCDR3, LCDR1, LCDR2 and LCDR3 for said antibody comprising the amino acid sequences as set forth in SEQ ID NO: 81, SEQ ID NO: 88, SEQ ID NO: 53, SEQ ID NO: 93, SEQ ID NO: 100, and SEQ ID NO: 109, respectively. In some embodiments, said HCDR1, HCDR2, HCDR3, LCDR1, LCDR2 and LCDR3 for said antibody comprising the amino acid sequences as set forth in SEQ ID NO: 81, SEQ ID NO: 89, SEQ ID NO: 53, SEQ ID NO: 93, SEQ ID NO: 100, and SEQ ID NO: 109, respectively. In some embodiments, said HCDR1, HCDR2, HCDR3, LCDR1, LCDR2 and LCDR3 for said antibody comprising the amino acid sequences as set forth in SEQ ID NO: 81, SEQ ID NO: 84, SEQ ID NO: 55, SEQ ID NO: 93, SEQ ID NO: 100, and SEQ ID NO: 109, respectively. In some embodiments, said HCDR1, HCDR2, HCDR3, LCDR1, LCDR2 and LCDR3 for said antibody comprising the amino acid sequences as set forth in SEQ ID NO: 81, SEQ ID NO: 84, SEQ ID NO: 56, SEQ ID NO: 93, SEQ ID NO: 100, and SEQ ID NO: 109, respectively. In some embodiments, said HCDR1, HCDR2, HCDR3, LCDR1, LCDR2 and LCDR3 for said antibody comprising the amino acid sequences as set forth in SEQ ID NO: 81, SEQ ID NO: 88, SEQ ID NO: 55, SEQ ID NO: 93, SEQ ID NO: 100, and SEQ ID NO: 109, respectively. In some embodiments, said HCDR1, HCDR2, HCDR3, LCDR1, LCDR2 and LCDR3 for said antibody comprising the amino acid sequences as set forth in SEQ ID NO: 81, SEQ ID NO: 88, SEQ ID NO: 56, SEQ ID NO: 93, SEQ ID NO: 100, and SEQ ID NO: 109, respectively. In some embodiments, said HCDR1, HCDR2, HCDR3, LCDR1, LCDR2 and LCDR3 for said antibody comprising the amino acid sequences as set forth in SEQ ID NO: 81, SEQ ID NO: 89, SEQ ID NO: 55, SEQ ID NO: 93, SEQ ID NO: 100, and SEQ ID NO: 109, respectively. In some embodiments, said HCDR1, HCDR2, HCDR3, LCDR1, LCDR2 and LCDR3 for said antibody comprising the amino acid sequences as set forth in SEQ ID NO: 81, SEQ ID NO: 89, SEQ ID NO: 56, SEQ ID NO: 93, SEQ ID NO: 100, and SEQ ID NO: 109, respectively.

A skilled artisan would appreciate that an “IL-8 binding antibody” encompasses in its broadest sense an antibody that specifically binds an antigenic determinant of an IL-8 polypeptide. The skilled artisan would appreciate that specificity for binding to IL-8, reflects that the binding is selective for the IL-8 antigen and can be discriminated from unwanted or nonspecific interactions. In certain embodiments, an IL-8 binding antibody comprises an antibody fragment or fragments.

In some embodiments, an antigenic determinant comprises an IL-8 epitope. The term “epitope” includes any determinant, in certain embodiments, a polypeptide determinant, capable of specific binding to an anti-IL-8 binding domain. An epitope is a region of an antigen that is bound by an antibody or an antigen-binding fragment thereof. In some embodiments, an IL-8 antigen-binding fragment of an antibody comprises a heavy chain variable region, a light chain variable region, or a combination thereof as described herein.

In certain embodiments, epitope determinants include chemically active surface groupings of molecules such as amino acids, sugar side chains, phosphoryl or sulfonyl, and may in certain embodiments have specific three-dimensional structural characteristics, and/or specific charge characteristics. In certain embodiments, an IL-8 binding antibody is said to specifically bind an IL-8 epitope when it preferentially recognizes IL-8 in a complex mixture of proteins and/or macromolecules.

In some embodiments, an IL-8 binding antibody is said to specifically bind an IL-8 epitope when the equilibrium dissociation constant is ≤10⁻⁵, 10⁻⁶, or 10⁻⁷ M. In some embodiments, the equilibrium dissociation constant may be ≤10⁻⁸ M or 10⁻⁹ M. In some further embodiments, the equilibrium dissociation constant may be ≤10⁻¹⁰ M, 10⁻¹¹ M, or 10⁻¹² M. In some embodiments, the equilibrium dissociation constant may be in the range of ≤10⁻⁵ M to 10⁻¹²M.

An antibody binding domain can be a fragment of an antibody or a genetically engineered product of one or more fragments of the antibody, which fragment is involved in specifically binding with the antigen. By “specifically binding” is meant that the binding is selective for the antigen of interest, for example for IL-8 in embodiments described herein and can be discriminated from unwanted or nonspecific interactions. As used herein, the term “IL-8 binding antibody” may in certain embodiments, encompass complete immunoglobulin structures, fragments thereof, or domains thereof.

In some embodiments, an IL-8 antibody described herein binds to human IL-8. In some embodiments, an IL-8 antibody described herein binds to cynomolgus IL-8. In some embodiments, an IL-8 antibody described herein binds to both a human and a cynomolgus IL-8. In some embodiments, an IL-8 binding antibody described herein inhibits binding of IL-8 to a CXCR1 receptor. In some embodiments, an IL-8 binding antibody described herein reduces binding of IL-8 to a CXCR1 receptor. In some embodiments, an IL-8 binding antibody described herein inhibits binding of IL-8 to a CXCR2 receptor. In some embodiments, an IL-8 binding antibody described herein reduces binding of IL-8 to a CXCR2 receptor. In some embodiments, an IL-8 binding antibody described herein inhibits binding of IL-8 to CXCR1 and CXCR2 receptors. In some embodiments, an IL-8 binding antibody described herein reduces binding of IL-8 to CXCR1 and CXCR2 receptors.

In some embodiments, an IL-8 binding antibody described herein inhibits IL-8 induced internalization of a CXCR1 receptor. In some embodiments, an IL-8 binding antibody described herein reduces IL-8 induced internalization of a CXCR1 receptor. In some embodiments, an IL-8 binding antibody described herein inhibits IL-8 induced internalization of a CXCR2 receptor. In some embodiments, an IL-8 binding antibody described herein reduces IL-8 induced internalization of a CXCR2 receptor. In some embodiments, an IL-8 binding antibody described herein inhibits IL-8 induced internalization of CXCR1 and CXCR2 receptors. In some embodiments, an IL-8 binding antibody described herein reduces IL-8 induced internalization of a CXCR1 and CXCR2 receptors.

In some embodiments, an IL-8 binding antibody described herein inhibits IL-8 induced NFκB activity. In some embodiments, an IL-8 binding antibody described herein reduces IL-8 induced NFκB activity. In some embodiments, an IL-8 binding antibody described herein inhibits IL-8 activated neutrophil migration. In some embodiments, an IL-8 binding antibody described herein reduces IL-8 neutrophil migration. In some embodiments, an IL-8 binding antibody described herein inhibits IL-8 activated monocyte migration. In some embodiments, an IL-8 binding antibody described herein reduces IL-8 activated monocyte migration.

In some embodiments, an IL-8 binding antibody described herein inhibits IL-8 induced AKT and ERK phosphorylation. In some embodiments, an IL-8 binding antibody described herein reduces IL-8 induced AKT and ERK phosphorylation in neutrophils.

In some embodiments, an IL-8 binding antibody described herein reduces inflammation. In some embodiments, an IL-8 binding antibody described herein eliminates inflammation. In some embodiments, an IL-8 binding antibody described herein reduces swelling in joints. In some embodiments, an IL-8 binding antibody described herein reduces swelling in a moving joint selected from ball and socket, saddle, hinge, condyloid, pivot and gliding joint. In some embodiments, an IL-8 binding antibody described herein reduces swelling in joints selected from a knee, finger, shoulder, elbow, wrist, ankle, toe, or hip joint, or any combination thereof. In some embodiments, an IL-8 binding antibody described herein reduces inflammation. In some embodiments, an IL-8 binding antibody described herein reduces inflammation in a joint. In some embodiments, an IL-8 binding antibody described herein reduces inflammation in a moving joint selected from ball and socket, saddle, hinge, condyloid, pivot and gliding joint. In some embodiments, an IL-8 binding antibody described herein reduces swelling in a joint selected from a knee, finger, shoulder, elbow, wrist, ankle, toe, or hip joint, or any combination thereof.

Examples of antibody binding domains include, without limitation, a complementarity determining region (CDR), a variable region (Fv), a VH domain, a light chain variable region (VL), a heavy chain, a light chain, a single chain variable region (scFv), and a Fab fragment. A skilled artisan would appreciate that an scFv is not actually a fragment of an antibody, but instead is a fusion polypeptide comprising the variable heavy chain (VH) and variable light chain (VL) regions of an immunoglobulin, connected by a short linker peptide of for example but not limited to ten to about 25 amino acids. The skilled artisan would also appreciate that the term “Fab” with regard to an antibody, generally encompasses that portion of the antibody consisting of a single light chain (both variable and constant regions) bound to the variable region and first constant region of a single heavy chain by a disulfide bond.

In some embodiments, an antibody encompasses whole antibody molecules, including monoclonal, polyclonal and multispecific (e.g., bispecific) antibodies. In some embodiments, an antibody encompasses an antibody fragment or fragments that retain binding specificity including, but not limited to, variable heavy chain (VH) fragments, variable light chain (VL) fragments, Fab fragments, F(ab′)2 fragments, scFv fragments, Fv fragments, minibodies, diabodies, triabodies, and tetrabodies (see, e.g., Hudson and Souriau, Nature Med. 9: 129-134 (2003) (hereby incorporated by reference in their entirety)). Also encompassed are humanized, primatized, and chimeric antibodies.

A skilled artisan would appreciate that an “isolated IL-8 binding antibody”, in certain embodiments, encompasses an antibody that (1) is free of at least some other proteins with which it would typically be found in nature or with which it would typically be found during synthesis thereof, (2) is essentially free of other non-identical IL-8 binding antibodies from the same source, (3) may be expressed recombinantly by a cell, (4) has been separated from at least about 50 percent of polynucleotides, lipids, carbohydrates, or other materials with which it is associated in during synthesis, or (5) does not occur in nature, or a combination thereof. Such an isolated antibody may be encoded by genomic DNA, cDNA, mRNA or other RNA, of may be of synthetic origin, or any combination thereof. In certain embodiments, the isolated antibody is substantially free from proteins or polypeptides or other contaminants that would interfere with its use (therapeutic, diagnostic, prophylactic, research or otherwise). As used throughout, the terms “IL-8 antibody”, “IL-8 binding antibody”, and the like, may be used interchangeably having all the same meanings and qualities.

In some embodiments, an IL-8 antibody comprises a recombinant antibody. In some embodiments, an IL-8 antibody comprises a humanized antibody. In some embodiments, an IL-8 antibody comprises an engineered antibody. In certain embodiments, an engineered antibody comprises improved binding compared to available antibodies. In some embodiments, an engineered antibody comprises improved association and dissociation constants (Kₒₙ and K_{off}), compared to available other IL-8 binding antibodies. In some embodiments, an engineered antibody comprises improved stability compared with available IL-8 binding antibodies.

In certain embodiments, the present disclosure provides polypeptides comprising the VH and VL domains which could be dimerized under suitable conditions. For example, the VH and VL domains may be combined in a suitable buffer and dimerized through appropriate interactions such as hydrophobic interactions. In another embodiment, the VH and VL domains may be combined in a suitable buffer containing an enzyme and/or a cofactor which can promote dimerization of the VH and VL domains. In another embodiment, the VH and VL domains may be combined in a suitable vehicle that allows them to react with each other in the presence of a suitable reagent and/or catalyst.

In certain embodiments, the VH and VL domains may be contained within longer polypeptide sequences, that may include for example but not limited to, constant regions, hinge regions, linker regions, Fc regions, or disulfide binding regions, or any combination thereof. A constant domain is an immunoglobulin fold unit of the constant part of an immunoglobulin molecule, also referred to as a domain of the constant region (e.g. CH1, CH2, CH3, CH4, Ck, Cl).

In some embodiments, an anti-IL-8 antibody comprises a mutated immunoglobulin. Examples of mutated immunoglobulins include immunoglobulins where the Fc portion has been engineered. The cellular immune response occurs mostly due to the interactions between the antibody and Fc gamma receptors (FcTRs). Non-limiting examples of immunoglobulins wherein the Fc portion of an immunoglobulin has been engineered is provided in Wang et al., (2018) Protein Cell, 9(1):63-73 (See Table 1 of Wang et al.), incorporated herein in full. Examples of mutated immunoglobulins, wherein binding of an IgG with cellular cytotoxicity (ADCC) components is altered may be found for example in Xu D, Alegre M L, Varga S S, Rothermel A L, Collins A M, Pulito V L, et al. In vitro characterization of five humanized OKT3 effector function variant antibodies. Cell Immunol. (2000) 200:16-26, incorporated herein in full. In some embodiments, an anti-IL8 immunoglobulin comprises an engineered Fc portion such that the interaction between the antibody and an Fc gamma receptor is increased, decreased, or eliminated.

In some embodiments, a mutated anti-IL-8 IgG comprises an IgG1, wherein the Fc region is engineered. In some embodiments, a mutated anti-IL-8 IgG comprises an IgG2, wherein the Fc region is engineered. anti-IL-8 In some embodiments, a mutated anti-IL-8 IgG comprises an IgG4, wherein the Fc region is engineered. In some embodiments, Fc variants comprising mutations within an Fc region exhibit reduced effector function. In certain embodiments, mutations within an Fc region of an antibody abolishes immune effector functions of the antibody. In some embodiments, the Fc portion of an antibody described herein is engineered wherein said engineered antibody comprises improved efficacy or safety or both relative to the human IgG isotype.

In some embodiments, a mutation comprises a missense mutation (substituting at least one amino acid for another), a nonsense mutation (substituting a STOP codon such that translation of the antibody polypeptide is halted early), an insertion mutation (inserting at least one amino acid) or a deletion mutation (deleting at least one amino acid).

In some embodiments, alteration of glycosylation of an IgG affects antibody activity. In some embodiments, a mutated anti-IL-8 IgG comprises an altered glycosylation pattern within the Fc portion of the antibody. In some embodiments, a mutated anti-IL-8 IgG comprising an altered glycosylation pattern has enhanced effector function. In some embodiments, a mutated anti-IL-8 antibody comprises an afucosylated antibody. In some embodiments, a mutated anti-IL-8 IgG comprising an altered glycosylation pattern has decreased effector function.

In some embodiments, modulation of effector function of an anti-IL-8 antibody comprises mutating the Fc region of the antibody such that the antibody-dependent cellular cytotoxicity (ADCC) response is enhanced. In some embodiments, modulation of effector function of an anti-IL-8 antibody comprises mutating the Fc region of the antibody such that the antibody no longer binds ADCC components, in which case the ADCC response is reduced or eliminated. For example, but not limited to, an IgG comprising L234A/L235A (LALA) mutations cannot bind the Fc receptor.

In some embodiments, an anti-IL-8 antibody comprises a mutated Fc region of the antibody such that the antibody-dependent cellular phagocytosis (ADCP) response is enhanced. In some embodiments, an anti-IL-8 antibody comprises a mutated Fc region of the antibody such that the antibody complement-dependent cytotoxicity (CDC) response is enhanced.

In certain embodiments, mutations within an Fc region of an IL-8 antibody increases binding to an Fc gamma receptor. In certain embodiments, mutations within an Fc region of an IL-8 antibody decreases binding to an Fc gamma receptor. In certain embodiments, mutations within an Fc region of an IL-8 antibody eliminates binding to an Fc gamma receptor.

In certain embodiments, mutations within an Fc region of an IL-8 antibody increases the half-life of the IgG. In certain embodiments, mutations within an Fc region of an IL-8 antibody increases co-engagement of antibody with a target antigen and with the Fc gamma receptor. In certain embodiments, mutations within an Fc region of an IL-8 antibody decrease co-engagement of antibody with a target antigen and with the Fc gamma receptor.

In some embodiments, an isolated anti-IL-8 antibody described here, comprises an IgG, an Fv, an scFv, an Fab, an F(ab′)2, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody, or a combination thereof. In some embodiments, an IgG can be of the subclass IgG1, IgG2, IgG3, or an IgG4. In certain embodiments, an IgG comprises an IgG1. In certain embodiments, an IgG comprises an IgG2. In certain embodiments, an IgG comprises an IgG3. In certain embodiments, an IgG comprises an IgG4.

In some embodiments, disclosed herein is an isolated polynucleotide sequence encoding a heavy chain variable region (VH) of an anti-IL-8 antibody and a light chain variable region (VL) of the anti-IL-8 antibody, wherein the amino acid sequence of the VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 2 and 3. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 4 and 5. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 6 and 7. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 8 and 9. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 10 and 11. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 12 and 13. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 14 and 15. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 16 and 17. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 18 and 19. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 20 and 21. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 22 and 23. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 24 and 25. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 26 and 27. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 28 and 29. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 30 and 31. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 32 and 33. In some embodiments, disclosed herein is an isolated polynucleotide sequence encoding a VH of an anti-IL-8 antibody and a VL of the anti-IL-8 antibody, wherein the amino acid sequence of the VH-VL pair comprise the paired sequences set forth in SEQ ID NOs: 34 and 35.

In some embodiments, described herein is an isolated anti-IL-8 antibody comprising an antibody antigen-binding domain comprising a heavy chain variable region (VH) and a light chain variable region (VL), wherein the polynucleotide sequences encoding the VH-VL pair are selected from the paired sequences set forth in SEQ ID NOs: 113 and 114, SEQ ID NOs: 115 and 116, SEQ ID NOs: 117 and 118, SEQ ID NOs: 119 and 120, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39. In some embodiments, the nucleotide sequences encoding a VH-VL pair is set forth in SEQ ID NOs: 113 and 114. In some embodiments, the nucleotide sequence encoding a VH-VL pair is set forth in SEQ ID NOs: 115 and 116. In some embodiments, the nucleotide sequences encoding a VH-VL pair is set forth in SEQ ID NOs: 117 and 118. In some embodiments, the nucleotide sequences encoding a VH-VL pair is set forth in SEQ ID NOs: 119 and 120. In some embodiments, the nucleotide sequences encoding a VH-VL pair is set forth in SEQ ID NOs: 121 and 122. In some embodiments, the nucleotide sequences encoding a VH-VL pair is set forth in SEQ ID NOs: 123 and 124. In some embodiments, the nucleotide sequences encoding a VH-VL pair is set forth in SEQ ID NOs: 125 and 126. In some embodiments, the nucleotide sequences encoding a VH-VL pair is set forth in SEQ ID NOs: 127 and 128. In some embodiments, the nucleotide sequences encoding a VH-VL pair is set forth in SEQ ID NOs: 129 and 130. In some embodiments, the nucleotide sequences encoding a VH-VL pair is set forth in SEQ ID NOs: 131 and 132. In some embodiments, the nucleotide sequences encoding a VH-VL pair is set forth in SEQ ID NOs:133 and 134. In some embodiments, the nucleotide sequences encoding a VH-VL pair is set forth in SEQ ID NOs: 135 and 136. In some embodiments, the nucleotide sequences encoding a VH-VL pair is set forth in SEQ ID NOs: 137 and 138. In some embodiments, the nucleotide sequences encoding a VH-VL pair is set forth in SEQ ID NOs: 139 and 140. In some embodiments, the nucleotide sequences encoding a VH-VL pair is set forth in SEQ ID NOs: 141 and 142. In some embodiments, the nucleotide sequences encoding a VH-VL pair is set forth in SEQ ID NOs: 36 and 37. In some embodiments, the nucleotide sequences encoding a VH-VL pair is set forth in SEQ ID NOs: 38 and 39.

One skilled in the art would appreciate that as used herein, the term “polynucleotide sequence” and “nucleotide sequence” may in certain embodiments, be used interchangeably having all the same meanings and qualities.

In certain embodiments, disclosed herein is an isolated polynucleotide sequence encoding a heavy chain variable region (VH) of an anti-IL-8 antibody comprising the complementarity determining regions (HCDR) of said VH as set forth in Table 1F and a light chain variable region (VL) of an anti-IL-8 antibody comprising the complementarity determining regions (LCDR) of said VL as set forth in Table 1F, wherein said heavy chain variable region comprises heavy chain complementarity determining region (HCDR) 1, HCDR2 and HCDR3, and said light chain variable region comprises light chain complementarity determining region (LCDR) 1, LCDR2 and LCDR3, wherein said HCDR1, HCDR2, HCDR3, LCDR1, LCDR2 and LCDR3 for each of said VH and VL comprise the amino acid sequences as set forth in Table 1F. In some embodiments, disclosed herein is an isolated polynucleotide sequence encoding a heavy chain variable region (VH) of an anti-IL-8 antibody comprising the complementarity determining regions (HCDR) of said VH as set forth in Table 1F and a light chain variable region (VL) of an anti-IL-8 antibody comprising the complementarity determining regions (LCDR) of said VL as set forth in Table 1F, said heavy chain variable region having heavy chain complementarity determining region (HCDR) 1, HCDR2 and HCDR3, and said light chain variable region having light chain complementarity determining region (LCDR) 1, LCDR2 and LCDR3, wherein said HCDR1, HCDR2, HCDR3, LCDR1, LCDR2 and LCDR3 for each of said antibody comprise the amino acid sequences as set forth:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109.

In certain embodiments, disclosed herein is an isolated polynucleotide sequence encoding a heavy chain variable region (VH) of an anti-IL-8 antibody comprising the complementarity determining regions (HCDR) of said VH, wherein the polynucleotide sequence is as set forth in Table 1G wherein the CDR regions are designated therein, and an isolated polynucleotide sequence encoding the light chain variable region (VL) of an anti-IL-8 antibody comprising the complementarity determining regions (LCDR) of said VL, said polynucleotide sequences as set forth in Table 1G, wherein the CDR regions are designated therein, wherein said heavy chain variable region comprises heavy chain complementarity determining region (HCDR) 1, HCDR2 and HCDR3, and said light chain variable region comprises light chain complementarity determining region (LCDR) 1, LCDR2 and LCDR3, wherein said HCDR1, HCDR2, HCDR3, LCDR1, LCDR2 and LCDR3 for each of said VH and VL comprise the nucleotide sequences as set forth in Table 1G.

In some embodiments, a polynucleotide sequence disclosed herein comprises two polynucleotide sequences, a first polynucleotide sequence encoding the VH of the anti-IL-8 antibody and a second polynucleotide sequence encoding the VL of the anti-IL-8 antibody. In some embodiments, a polynucleotide sequence encodes the VH and VL regions of an anti-IL-8 antibody comprising an IgG, an Fv, an scFv, an Fab, an F(ab′)2, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody, or a combination thereof. In some embodiments, a polynucleotide sequence encodes the VH and VL regions of an anti-IL-8 antibody comprising an IgG, wherein said IgG can be of the subclass IgG1, IgG2, IgG3, or IgG4.

In some embodiments, the present disclosure also provides a vector comprising the above-mentioned polynucleotide sequences. In some embodiments, a vector comprises two vectors, one comprising a polynucleotide encoding a VH and one comprising a polynucleotide encoding a VL. In view of the amino acid sequences disclosed herein, one of ordinary skill in the art would readily construct a vector or plasmid to encode for the amino acid sequences.

One skilled in the art would appreciate that the polynucleotides described herein, or fragments thereof, regardless of the length of the coding sequence itself, may be combined with other DNA sequences, such as promoters, polyadenylation signals, additional restriction enzyme sites, multiple cloning sites, other coding segments, and the like, such that their overall length may vary considerably. It is therefore contemplated that a nucleic acid fragment of almost any length may be employed, with the total length preferably being limited by the ease of preparation and use in the intended recombinant DNA protocol. For example, illustrative polynucleotide segments with total lengths of about 10,000, about 5000, about 3000, about 2,000, about 1,000, about 500, about 200, about 100, about 50 base pairs in length, and the like, (including all intermediate lengths) are contemplated to be useful.

In certain embodiments, the isolated polynucleotide is inserted into a vector. The term “vector” as used herein encompasses a vehicle into which a polynucleotide encoding a protein may be covalently inserted so as to bring about the expression of that protein and/or the cloning of the polynucleotide. The isolated polynucleotide may be inserted into a vector using any suitable methods known in the art, for example, without limitation, the vector may be digested using appropriate restriction enzymes and then may be ligated with the isolated polynucleotide having matching restriction ends.

Examples of suitable vectors include, without limitation, plasmids, phagemids, cosmids, artificial chromosomes such as yeast artificial chromosome (YAC), bacterial artificial chromosome (BAC), or P1-derived artificial chromosome (PAC), bacteriophages such as lambda phage or M13 phage, and animal viruses. Examples of categories of animal viruses useful as vectors include, without limitation, retrovirus (including lentivirus), adenovirus, adeno-associated virus, herpesvirus (e.g., herpes simplex virus), poxvirus, baculovirus, papillomavirus, and papovavirus (e.g., SV40). In some embodiments, said vector comprises an expression vector.

In some embodiments, an expression vector comprises a nucleic acid construct described herein. Suitable vectors can be chosen or constructed, containing appropriate regulatory sequences, including promoter sequences, terminator sequences, polyadenylation sequences, enhancer sequences, marker genes and other sequences as appropriate. Regulatory sequences may be operably linked to the nucleic acid sequence(s) comprised within a nucleic acid construct. Vectors may be plasmids, viral e.g. ‘phage, or phagemid, as appropriate. For further details see, for example, Molecular Cloning: a Laboratory Manual: 3rd edition, Sambrook and Russell, 2001, Cold Spring Harbor Laboratory Press. Many known techniques and protocols for manipulation of nucleic acid, for example in preparation of nucleic acid constructs, mutagenesis, sequencing, introduction of DNA into cells and gene expression, and analysis of proteins, are described in detail in Current Protocols in Molecular Biology, Second Edition, Ausubel et al. eds., John Wiley & Sons, 1988, Short Protocols in Molecular Biology: A Compendium of Methods from Current Protocols in Molecular Biology, Ausubel et al. eds., John Wiley & Sons, 4.sup.th edition 1999. The disclosures of Sambrook et al. and Ausubel et al. (both) are incorporated herein by reference.

The vector can be introduced to the host cell using any suitable methods known in the art, including, without limitation, DEAE-dextran mediated delivery, calcium phosphate precipitate method, cationic lipids mediated delivery, liposome mediated transfection, electroporation, microprojectile bombardment, receptor-mediated gene delivery, delivery mediated by polylysine, histone, chitosan, and peptides. Standard methods for transfection and transformation of cells for expression of a vector of interest are well known in the art.

For expression of the IL-8 antibody or components thereof, the vector may be introduced into a host cell to allow expression of the polypeptide within the host cell. The expression vectors may contain a variety of elements for controlling expression, including without limitation, promoter sequences, transcription initiation sequences, enhancer sequences, selectable markers, and signal sequences. These elements may be selected as appropriate by a person of ordinary skill in the art. In some embodiments, these elements may be considered “control” elements.

A skilled artisan would appreciate that the term “control sequence” may encompass polynucleotide sequences that can affect expression, processing or intracellular localization of coding sequences to which they are ligated or operably linked. The nature of such control sequences may depend upon the host organism. In particular embodiments, transcription control sequences for prokaryotes may include a promoter, ribosomal binding site, and transcription termination sequence. In other particular embodiments, transcription control sequences for eukaryotes may include promoters comprising one or a plurality of recognition sites for transcription factors, transcription enhancer sequences, transcription termination sequences and polyadenylation sequences. In certain embodiments, “control sequences” can include leader sequences and/or fusion partner sequences.

In some embodiments, for example but not limited to, the promoter sequences may be selected to promote the transcription of the polynucleotide in the vector. Suitable promoter sequences include, without limitation, T7 promoter, T3 promoter, SP6 promoter, beta-actin promoter, EF1a promoter, CMV promoter, and SV40 promoter. Enhancer sequences may be selected to enhance the transcription of the polynucleotide. Selectable markers may be selected to allow selection of the host cells inserted with the vector from those not, for example, the selectable markers may be genes that confer antibiotic resistance. Signal sequences may be selected to allow the expressed polypeptide to be transported outside of the host cell.

A vector may also include materials to aid in its entry into the cell, including but not limited to a viral particle, a liposome, or a protein coating.

In some embodiments, an expression vector comprises an isolated polynucleotide sequence encoding an IL-8 antibody or a component thereof, for example but not limited to a VH domain, a VL domain, a combined VH-VL domain as may be present in Fab elements, F(ab′)2 elements, an IgG, an Fv, or an scFv. In some embodiments, an expression vector comprises a polynucleotide sequence encoding an IL-8 VH domain or VL domain, or a combination thereof, wherein the polynucleotide sequence is selected from those presented in Table 1G. In some embodiments, an expression vector comprises a polynucleotide sequence encoding an IL-8 VH domain or VL domain, or a combination thereof, wherein the polynucleotide sequence(s) is selected from the sequences set forth in SEQ ID NOs: 113-142 and 36-39. In some embodiments, an isolated polynucleotide sequence encodes a component of an anti-IL-8 antibody component of a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody, or a combination thereof, as described above. IL-8 binding domains and the components thereof have been described in detail above.

In some embodiments, an expression vector comprises an isolated polynucleotide sequence encoding a VH domain. In some embodiments, an expression vector comprises an isolated nucleic acid sequence encoding a VL domain. In some embodiments, an expression vector comprises an isolated nucleic acid sequence encoding a VH and a VL domain. In some embodiments, an expression vector comprises an isolated nucleic acid sequence encoding set of CDR's of a VH region. In some embodiments, an expression vector comprises an isolated nucleic acid sequence encoding set of CDR's of a VL region. In some embodiments, an expression vector comprises an isolated nucleic acid sequence encoding set of CDR's of a VH region and a VL region. In some embodiments, the CDR regions of the VH and VL regions are those indicated in Table 1G.

In another embodiment, the present disclosure also provides a host cell comprising the vector provided herein. Depending on the uses and experimental conditions, one of skill in the art would readily employ a suitable host cell to carry and/or express the above-mentioned polynucleotide sequences.

For cloning of the polynucleotide, the vector may be introduced into a host cell (an isolated host cell) to allow replication of the vector itself and thereby amplify the copies of the polynucleotide contained therein. The cloning vectors may contain sequence components generally include, without limitation, an origin of replication, promoter sequences, transcription initiation sequences, enhancer sequences, and selectable markers. These elements may be selected as appropriate by a person of ordinary skill in the art. For example, the origin of replication may be selected to promote autonomous replication of the vector in the host cell.

In certain embodiments, the present disclosure provides isolated host cells containing the vector provided herein. The host cells containing the vector may be useful in expression or cloning of the polynucleotide(s) contained in the vector.

In some embodiments, a recombinant host cell comprises one or more constructs as described above. A polynucleotide encoding any CDR or set of CDR's or VH domain or VL domain or antibody antigen-binding site or antibody molecule, for example but not limited to an IgG, an Fv, an scFv, an Fab, an F(ab′)₂, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody, or a combination thereof. In some embodiments, a host cell comprises one or more constructs as described above encoding an IgG subclass selected from an IgG1, IgG2, IgG3, and IgG4.

In some embodiments, disclosed herein is a method of production of the encoded product, which method comprises expression from the polynucleotide constructs. In some embodiments, a polynucleotide construct comprises a polynucleotide sequence selected from SEQ ID NO: 113-142 and 36-39 or the CDR portions thereof. Expression may in some embodiments, be achieved by culturing under appropriate conditions recombinant host cells containing the nucleic acid construct. Following production by expression, an antibody or an IL-8 antigen-binding fragment thereof, may be isolated and/or purified using any suitable technique, then used as appropriate, for example in methods of treatment as described herein.

In some embodiments, systems for cloning and expression of a polypeptide in a variety of different host cells are well known. Suitable host cells can include, without limitation, prokaryotic cells, fungal cells, yeast cells, or higher eukaryotic cells such as insect cells or mammalian cells.

Suitable prokaryotic cells for this purpose include, without limitation, eubacteria, such as Gram-negative or Gram-positive organisms, for example, Enterobactehaceae such as Escherichia, e.g., E. coli, Enterobacter, Erwinia, Klebsiella, Proteus, Salmonella, e.g., Salmonella typhimurium, Serratia, e.g., Serratia marcescans, and Shigella, as well as Bacilli such as B. subtilis and B. licheniformis, Pseudomonas such as P. aeruginosa, and Streptomyces.

The expression of antibodies and antigen-binding fragments in prokaryotic cells such as E. coli is well established in the art. For a review, see for example Pluckthun, A. Bio/Technology 9: 545-551 (1991). Expression in eukaryotic cells in culture is also available to those skilled in the art as an option for production of antibodies or antigen-binding fragments thereof, see recent reviews, for example Ref, M. E. (1993) Curr. Opinion Biotech. 4: 573-576; Trill J. J. et al. (1995) Curr. Opinion Biotech 6: 553-560.

Suitable fungal cells for this purpose include, without limitation, filamentous fungi and yeast. Illustrative examples of fungal cells include, Saccharomyces cerevisiae, common baker's yeast, Schizosaccharomyces pombe, Kluyveromyces hosts such as, eg., K. lactis, K. fragilis (ATCC 12,424), K. bulgaricus (ATCC 16,045), K. wickeramii (ATCC 24,178), K. waltii (ATCC 56,500), K. drosophilarum (ATCC 36,906), K. thermotolerans, and K. marxianus; yarrowia (EP 402,226); Pichia pastoris (EP 183,070); Candida; Trichoderma reesia (EP 244,234); Neurospora crassa; Schwanniomyces such as Schwanniomyces occidentalis; and filamentous fungi such as, e.g., Neurospora, Penicillium, Tolypocladium, and Aspergillus hosts such as A. nidulans and A. niger.

Higher eukaryotic cells, in particular, those derived from multicellular organisms can be used for expression of glycosylated VH and VL domains, as provided herein. Suitable higher eukaryotic cells include, without limitation, invertebrate cells and insect cells, and vertebrate cells. Examples of invertebrate cells include plant and insect cells. Numerous baculoviral strains and variants and corresponding permissive insect host cells from hosts such as Spodoptera frugiperda (caterpillar), Aedes aegypti (mosquito), Aedes albopictus (mosquito), Drosophila melanogaster (fruit fly), and Bombyx mori have been identified. A variety of viral strains for transfection are publicly available, e.g., the K-1 variant of Autographa californica NPV and the Bm-5 strain of Bombyx mori NPV, and such viruses may be used as the virus herein as described herein, particularly for transfection of Spodoptera frugiperda cells. Plant cell cultures of cotton, corn, potato, soybean, petunia, tomato, and tobacco can also be utilized as hosts. Mammalian cell lines available in the art for expression of a heterologous polypeptide include Chinese hamster ovary (CHO) cells, HeLa cells, baby hamster kidney cells, NS0 mouse melanoma cells, YB2/0 rat myeloma cells, human embryonic kidney cells, human embryonic retina cells and many others. Non-limiting examples of vertebrate cells include, mammalian host cell lines such as monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); ExpiCHO-S™ cells (ThermoFisher Scientific cat. #A29133); mouse Sertoli cells (TM4, Mather, Biol. Reprod. 23:243-251 (1980)); monkey kidney cells (CV1 ATCC CCL 70); African green monkey kidney cells (VERO-76, ATCC CRK-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2).

A non-limiting example of an expression system well known in the art is the Lonza (USA) GS Gene Expression System®. In some embodiments, a vector encoding a polypeptide described herein comprises a GS® vector of Lonza (USA), for example but not limited to pXC-IgG1zaDK (based on pXC-18.4) and pXC-Kappa (based on pXC-17.4). These GS® vectors and other similar vectors known in the art, include a range of vector choices comprising Universal base vectors, IgG constant region vectors, IgG site-specific conjugation vectors, pXC Multigene vectors, and GS piggyBac™ vectors (+transposase). In some embodiments, a host cell from which an encoded polypeptide described herein may be expressed comprises a GS Xceed® CHOK1SV GS-KO® cell line or other similar cell known known in the art or created for the purpose of optimizing protein expression. In some embodiments, the combination of vector and host cell optimizes expression of IL-8 antibody polypeptides or IL-8 binding fragments thereof.

In some embodiments, provided herein is a host cell containing nucleic acid as disclosed herein. Such a host cell may be in vitro and may be in culture. Such a host cell may be in vivo. In vivo presence of the host cell may allow intracellular expression of IL-8 binding antibodies described herein, as “intrabodies” or intracellular antibodies. Intrabodies may be used for gene therapy.

In certain embodiments, the host cells comprise a first vector encoding a first polypeptide, e.g., a VH domain, and a second vector encoding a second polypeptide, e.g., a VL domain. In certain embodiments, the host cells comprise a vector encoding a first polypeptide, e.g., a VH domain, and a second polypeptide, e.g., a VL domain.

In certain embodiments, the host cells comprise a first vector encoding a VH domain and a second vector encoding a VL domain. In certain embodiments, the host cells comprise a single vector encoding a VH domain and a VL domain.

In some embodiments, an isolated cell comprises an isolated nucleic acid sequence, as disclosed herein. In some embodiments, an isolated cell comprises two isolated nucleic acid sequences as disclosed herein, wherein one nucleic acid encodes a VH domain and the other nucleic acid encodes a VL domain. In some embodiments, an isolated cell comprises a single isolated nucleic acid sequences as disclosed herein, that encodes a VH domain and a VL domain.

In certain embodiments, a first vector and a second vector may or may not be introduced simultaneously. In certain embodiments, the first vector and the second vector may be introduced together into the host cell. In certain embodiments, the first vector may be introduced first into the host cell, and then the second vector may be introduced. In certain embodiments, the first vector may be introduced into the host cell, which is then established into a stable cell line expressing the first polypeptide, and then the second vector may be introduced into the stable cell line.

The introduction may be followed by causing or allowing expression from the nucleic acid, e.g. by culturing host cells under conditions for expression of the gene. In certain embodiments, the present disclosure provides methods of expressing the polypeptide provided herein, comprising culturing the host cell containing the vector under conditions in which the inserted polynucleotide in the vector is expressed.

In some embodiments, the nucleic acid is integrated into the genome (e.g. chromosome) of the host cell. Integration may be promoted by inclusion of sequences which promote recombination with the genome, in accordance with standard techniques. In some embodiments, the nucleic acid construct is not integrated into the genome and the vector is episomal.

In some embodiments, disclosed herein is a method which comprises using a construct as stated above in an expression system in order to express an IL-8 binding antibody or fragment thereof, as described herein above.

Suitable conditions for expression of the polynucleotide may include, without limitation, suitable medium, suitable density of host cells in the culture medium, presence of necessary nutrients, presence of supplemental factors, suitable temperatures and humidity, and absence of microorganism contaminants. A person with ordinary skill in the art can select the suitable conditions as appropriate for the purpose of the expression.

In some embodiments, IL-8 binding antibodies described herein may be prepared and isolated and/or purified, in substantially pure or homogeneous form. In some embodiments, disclosed herein is a method of producing an anti-IL-8 antibody comprising a heavy chain variable region (VH) and a light chain variable region (VH), comprises the step of culturing a host cell under conditions conducive to expressing a vector in said host cell, thereby expressing a polynucleotide sequence comprised in the vector and thereby producing an anti-IL-8 antibody or an IL-8 antigen binding domain thereof. In some embodiments, disclosed herein is a method of producing an anti-IL-8 antibody comprising a heavy chain variable region (VH) and a light chain variable region (VH), comprises the step of culturing a host cell comprising a vector comprising an isolated polynucleotide sequence encoding the heavy chain variable region (VH) of an anti-IL-8 antibody and the light chain variable region (VL) of the anti-IL-8 antibody, wherein the amino acid sequence of the VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35; under conditions conducive to expressing a vector in said host cell, thereby expressing a polynucleotide sequence comprised in the vector and thereby producing an anti-IL-8 antibody comprising a VH and VL or an IL-8 antigen binding domain thereof. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 2 and 3. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 4 and 5.

In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 6 and 7. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 8 and 9. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 10 and 11. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 12 and 13. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 14 and 15. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 16 and 17. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 18 and 19. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 20 and 21. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 22 and 23. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 24 and 25. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 26 and 27. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 28 and 29. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 30 and 31. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 32 and 33. In some embodiments, a method of producing an anti-IL-8 antibody comprises the step of culturing a host cell comprising a vector comprising an isolated polynucleotide sequence encoding the VH and the VL of an anti-IL-8 antibody, wherein the amino acid sequence of the VH-VL pair comprise the paired sequences set forth in SEQ ID NOs: 34 and 35.

In some embodiments, disclosed herein is a method of producing an anti-IL-8 antibody comprising a heavy chain variable region (VH) and a light chain variable region (VH), comprises the step of culturing a host cell comprising a vector comprising an isolated polynucleotide sequence encoding the heavy chain variable region (VH) of an anti-IL-8 antibody and the light chain variable region (VL) of the anti-IL-8 antibody, wherein the polynucleotide sequences encoding the VH-VL pair are selected from the paired sequences set forth in SEQ ID NOs: 113 and 114, SEQ ID NOs: 115 and 116, SEQ ID NOs: 117 and 118, SEQ ID NOs: 119 and 120, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39; under conditions conducive to expressing a vector in said host cell, thereby expressing a polynucleotide sequence comprised in the vector and thereby producing an anti-IL-8 antibody comprising a VH and VL or an IL-8 antigen binding domain thereof. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 113 and 114. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 115 and 116. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 117 and 118. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 119 and 120. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 121 and 122. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 123 and 124. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 125 and 126. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 127 and 128. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 129 and 130. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 131 and 132. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 133 and 134. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 135 and 136. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 137 and 138. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 139 and 140. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 141 and 142. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 36 and 37. In some embodiments, a method of producing an anti-IL-8 antibody comprises the step of culturing a host cell comprising a vector comprising an isolated polynucleotide sequence encoding the VH and the VL of the anti-IL-8 antibody, wherein the polynucleotide sequences encoding the VH-VL comprise the paired sequences set forth in SEQ ID NOs: 38 and 39.

In some embodiments, disclosed herein is a method of producing an anti-IL-8 antibody comprising complementarity determining region (CDR) sequences as set forth in Table 1F, the method comprising the step of culturing a host cell comprising a vector comprising an isolated polynucleotide sequence encoding a heavy chain variable region (VH) of an anti-IL-8 antibody comprising the complementarity determining regions (HCDR) of said VH as set forth in Table 1F and a light chain variable region (VL) of an anti-IL-8 antibody comprising the complementarity determining regions (LCDR) of said VL as set forth in Table 1F, said heavy chain variable region having heavy chain complementarity determining region (HCDR) 1, HCDR2 and HCDR3, and said light chain variable region having light chain complementarity determining region (LCDR) 1, LCDR2 and LCDR3, wherein said HCDR1, HCDR2, HCDR3, LCDR1, LCDR2 and LCDR3 for said antibody comprise the amino acid sequences as set forth in:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109;
   under conditions conducive to expressing said vector in said host cell, and expressing said polynucleotide sequences comprised in said vector, thereby producing an anti-IL-8 antibody having complementarity determining region (CDR) sequences as set forth in Table 1F.

In some embodiments of a method for producing an IL-8 antibody, the antibody is produced in vivo. In some embodiments of a method for producing an IL-8 antibody, the antibody is produced in vitro. In some embodiments of a method for producing an IL-8 antibody, when the antibody is produced in vitro it may in a further step be isolated.

### Compositions of Use

The anti-IL-8 antibodies disclosed herein can be administered to a subject (e.g. a human or an animal) alone, or in combination with a carrier, i.e., a pharmaceutically acceptable carrier. By pharmaceutically acceptable is meant a material that is not biologically or otherwise undesirable, i.e., the material can be administered to a subject without causing any undesirable biological effects or interacting in a deleterious manner with any of the other components of the pharmaceutical composition in which it is contained. As would be well-known to one of ordinary skill in the art, the carrier is selected to minimize any degradation of the polypeptides disclosed herein and to minimize any adverse side effects in the subject. A skilled artisan would appreciate that the term “physiologically acceptable carrier, diluent or excipient”, may in some embodiments be used interchangeably with the term “pharmaceutically acceptable carrier” having all the same means and qualities.

The pharmaceutical compositions may be prepared by methodology well known in the pharmaceutical art.

In some embodiments, disclosed herein is a composition comprising an isolated anti-IL-8 antibody comprising an antibody antigen-binding domain comprising a heavy chain variable region (VH) and a light chain variable region (VL), wherein the amino acid sequences of a VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35; and a pharmaceutically acceptable carrier. In some embodiments, disclosed herein is a composition comprising an isolated anti-IL-8 antibody having complementarity determining region (CDR) sequences as set forth in Table 1F, wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, wherein said HCDR1, HCDR2, HCDR3, LCDR1, LCDR2 and LCDR3 for each of said antibody comprise the amino acid sequences as set forth in Table 1F; and a pharmaceutically acceptable carrier. In certain embodiments, a composition comprises an isolated anti-IL-8 antibody comprising CDR sequences as set forth in Table 1F, wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, said HCDR1, HCDR2, HCDR3, LCDR1, LCDR2 and LCDR3 for each antibody comprise the amino acid sequences as set forth:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109.

In some embodiments of a composition described herein, an antibody comprises an IgG, an Fv, an scFv, an Fab, an F(ab′)₂, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody, or a combination thereof. In certain embodiments, a composition comprises an anti-il-8 antibody comprising an IgG subclass selected from an IgG1, IgG2, IgG3, or an IgG4.

A skilled artisan would recognize that in some embodiments, the term “IL-8 binding antibody” may be used interchangeably with the term “drug” or “agent” having all the same meanings and qualities. In some embodiments, a drug comprising an IL-8 binding antibody comprises a pharmaceutical composition.

In some embodiments, an anti-IL-8 antibody disclosed herein can be administered to a subject (e.g. a human or an animal) alone or as part of a combination therapy with an additional therapeutic agent. In some embodiments, a composition described herein comprises an anti-IL-8 antibody in combination with an additional therapeutic agent. In some embodiments, an additional therapeutic agent comprises a chemotherapeutic agent, an anti-viral agent, an immune modulator, or an additional therapeutic antibody, or any combination thereof.

In some embodiments, an anti-IL-8 antibody as described herein, and an additional therapeutic agent are comprised in the same composition. In some embodiments, an anti-IL-8 antibody as described herein, and an additional therapeutic agent are comprised in different compositions. In some embodiments, administration of a combination of an anti-IL-8 antibody as described herein and an additional therapeutic agent, or composition(s) thereof are concurrent. In some embodiments, administration of a combination of an anti-IL-8 antibody as described herein and an additional therapeutic agent, or composition(s) thereof, comprises administration of an anti-IL-8 antibody or a composition thereof, prior to the administration of the additional therapeutic agent. In some embodiments, administration of a combination of an anti-IL-8 antibody as described herein and an additional therapeutic agent, or composition(s) thereof comprises administration of an anti-IL-8 antibody or a composition thereof, following administration of the additional therapeutic agent.

The above pharmaceutical compositions comprising an anti-IL-8 antibody disclosed herein can be administered (e.g., to a mammal, a cell, or a tissue) in any suitable manner depending on whether local or systemic treatment is desired. For example, the composition can be administered topically (e.g. ophthalmically, vaginally, rectally, intranasally, transdermally, and the like), orally, by inhalation, or parenterally (including by intravenous drip or subcutaneous, intracavity, intraperitoneal, intradermal, or intramuscular injection). Topical intranasal administration refers to delivery of the compositions into the nose and nasal passages through one or both of the nares. The composition can be delivered by a spraying mechanism or droplet mechanism, or through aerosolization. Delivery can also be directed to any area of the respiratory system (e.g., lungs) via intubation. Alternatively, administration can be intratumoral, e.g. local or intravenous injection.

If the composition is to be administered parenterally, the administration is generally by injection. Injectables can be prepared in conventional forms, either as liquid solutions or suspensions, solid forms suitable for suspension in liquid prior to injection, or as emulsions. Additionally, parental administration can involve preparation of a slow-release or sustained-release system so as to maintain a constant dosage.

### Methods of Use

The anti-IL-8 antibodies disclosed herein may be used in therapeutic methods. In some embodiments, an anti-IL-8 antibody can be used as an immunotherapeutic agent, for example, for treating a subject suffering from a disease, wherein the disease comprises a cancer or a tumor or a viral infection, as described herein. The anti-IL-8 antibodies disclosed herein may be used in therapeutic methods. In some embodiments, an anti-IL-8 antibody can be used as an immunotherapeutic agent, for example, for treating a subject suffering from a disease, wherein the disease comprises a cancer or a tumor or a viral infection or inflammation or a combination thereof, as described herein. In some embodiments, uses of an anti-IL-8 antibody described herein include use as an immunotherapeutic agent. In some embodiments, an anti-IL-8 antibody can be used as an immunotherapeutic agent for example, for responding to a cytokine release syndrome or a cytokine storm. In some embodiments, use of an anti-IL-8 antibody reduces the severity of a cytokine release syndrome or a cytokine storm. In some embodiments, use of an anti-IL-8 antibody reduces the severity of the impact on a subject of a cytokine release syndrome or a cytokine storm. In some embodiments, use of an anti-IL-8 antibody reduces the harmful impact of a cytokine release syndrome or a cytokine storm on organs or tissues affected by a viral infection. In some embodiments, an anti-IL-8 antibody described herein is used to treat a subject suffering from a cytokine release syndrome or a cytokine storm. In some embodiments, uses of an anti-IL-8 antibody described herein include use as an anti-inflammatory agent. In some embodiments, methods of use of an IL-8 antibody comprise treating inflammation in a subject in need, for example but not limited to for the reduction of swelling in a joint or joints of said subject.

In some embodiments, an anti-IL-8 antibody can be used as an immunotherapeutic agent, for example, for inhibiting tumor formation or growth, or a combination thereof. In some embodiments, an anti-IL-8 antibody can be used as an immunotherapeutic agent, for example, for differential inhibition of activation of immune cells as described herein. In some embodiments, an anti-IL-8 antibody can be used as immunotherapeutic agents, inhibiting neutrophil or monocyte activation, or a combination thereof. In some embodiments, an anti-IL-8 antibody can be used as immunotherapeutic agents, inhibiting neutrophil or monocyte migration into a tumor microenvironment, or a combination thereof. In some embodiments, an anti-IL-8 antibody can be used as immunotherapeutic agents, inhibiting neutrophil or monocyte migration into a viral infected microenvironment, or a combination thereof. In some embodiments, an anti-IL-8 antibody inhibits neutrophil and monocyte activation, and migration into tissues and organs affected by the viral infection. In some embodiments, these tissues and or organs are not infected with the virus but are impacted negatively through an indirect effect.

In some embodiments, an anti-IL-8 antibody blocks the ability of IL-8 to bind to its cell-surface receptor(s) and thereby interferes with the ability of IL-8 to transduce a cellular signal. In some embodiments, an anti-IL-8 antibody can be used as an immunotherapeutic agent, wherein the anti-IL-8 antibody inhibits cell growth, for example but not limited to inhibition of tumor cell growth. In some embodiments, an anti-IL-8 antibody can be used as an immunotherapeutic agent, wherein the anti-IL-8 antibody reduces growth, for example but not limited to reduction of tumor cell growth. In some embodiments, an anti-IL-8 antibody can be used as an immunotherapeutic agent, wherein the anti-IL-8 antibody inhibits cancer or tumor cell metastasis. In some embodiments, an anti-IL-8 antibody can be used as an immunotherapeutic agent, wherein the anti-IL-8 antibody reduces cancer or tumor cell metastasis.

In some embodiments, disclosed herein is a method of inhibiting tumor formation or growth or a combination thereof in a subject in need, the method comprising the step of administering to said subject an anti-IL-8 antibody as disclosed herein comprising an antibody antigen-binding domain comprising a heavy chain variable region (VH) and a light chain variable region (VL), wherein the amino acid sequences of a VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35, thereby inhibiting tumor formation or growth or a combination thereof in said subject. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 2 and 3. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 4 and 5. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 6 and 7. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 8 and 9. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 10 and 11. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 12 and 13. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 14 and 15. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 16 and 17. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 18 and 19. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 20 and 21. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 22 and 23. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 24 and 25. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 26 and 27. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 28 and 29. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 30 and 31. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 32 and 33. In some embodiments, a method of inhibiting tumor formation or growth or a combination thereof in a subject in need, comprises the step of administering to said subject an anti-IL-8 antibody comprising an antibody antigen-binding domain comprising a heavy chain variable region (VH) and a light chain variable region (VL), wherein the amino acid sequences of a VH-VL pair comprises the paired sequences set forth in SEQ ID NOs: 34 and 35.

In some embodiments, disclosed herein is a method of inhibiting tumor formation or growth or a combination thereof in a subject in need, comprising the step of administering to said subject an anti-IL-8 antibody having complementarity determining region (CDR) sequences as set forth in Table 1F, wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, wherein said HCDR1, HCDR2, HCDR3, LCDR1, LCDR2 and LCDR3 for each of said antibody comprises the amino acid sequences as set forth in Table 1F, thereby inhibiting tumor formation or growth or a combination thereof in said subject. In some embodiments, disclosed herein is a method of inhibiting tumor formation or growth or a combination thereof in a subject in need, comprising the step of administering to said subject an anti-IL-8 antibody having complementarity determining region (CDR) sequences as set forth in Table 1F, wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, said HCDR1, HCDR2, HCDR3, LCDR1, LCDR2 and LCDR3 for each of said antibody comprises the amino acid sequences as set forth:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109;
   thereby inhibiting tumor formation or growth or a combination thereof in said subject.

In some embodiments, a method of inhibiting tumor formation or growth or both inhibits tumor formation. In some embodiments, a method of inhibiting tumor formation or growth or both reduces the rate of tumor formation. In some embodiments, a method of inhibiting tumor formation or growth or both inhibits tumor growth. In some embodiments a method of inhibiting tumor formation or growth or both reduces the rate of tumor growth. In some embodiments, a method of inhibiting tumor formation or growth or both halts tumor growth. In some embodiments, a method of inhibiting tumor formation or growth or both inhibits tumor formation de novo and reduces the growth of a tumor. In some embodiments, a method of inhibiting tumor formation or growth or both reduces the rate of tumor formation de novo and reduces the growth of a tumor. In some embodiments, a method of inhibiting tumor formation or growth or both inhibits tumor formation de novo, inhibits the growth of a tumor, and inhibits metastasis. In some embodiments, a method of inhibiting tumor formation or growth or both reduces the rate of tumor formation de novo, reduces the growth of a tumor, and reduces the rate of tumor metastasis. In some embodiments, a method of inhibiting tumor formation or growth or both inhibits tumor metastasis. In some embodiments a method of inhibiting tumor formation or growth or both reduces the rate of tumor metastasis.

In certain embodiments, a method of inhibiting tumor formation or growth or both, inhibits neutrophil or monocyte activation, or a combination thereof within a tumor microenvironment. In certain embodiments, a method of inhibiting tumor formation or growth or both, reduces activation of neutrophils or monocytes, or a combination thereof, within a tumor microenvironment. A skilled artisan would appreciate that the terms “tumor microenvironment”, “cancer microenvironment”, “TME”, and “tumor milieu” may be used interchangeably having the same qualities and meanings and encompassing the microenvironment to tumor development. While the normal cellular microenvironment can inhibit malignant cell growth, the modifications that occur in the tumor microenvironment may synergistically support cell proliferation.

Tumors shape their microenvironment and support the development of both tumor cells and non-malignant cells. The tumor microenvironment affects angiogenesis by interfering with the signaling pathways required for cell recruitment and vascular construction. Endothelial progenitor cells (EPCs) that are recruited under hypoxic conditions for angiogenesis have been associated as well with metastasis. In addition. proteins such as IL-8 may be secreted by a tumor or cancer, wherein the presence of the secreted protein may modify the microenvironment by contributing growth factors and proteases that degrade the extracellular matrix and affect cell motility and adhesion.

In some embodiments, a method of inhibiting tumor formation or growth or both, decreases viability of pre-cancerous stem cells or tumor cells. Cell viability may be assessed by known techniques, such as trypan blue exclusion assays. Viability or conversely, toxicity, may also be measured based on cell viability, for example the viability of normal and cancerous cell cultures exposed to the anti-IL8 antibody may be compared. Toxicity may also be measured based on cell lysis, for example the lysis of normal and cancerous cell cultures exposed to the anti-IL-8 antibody may be compared. Cell lysis may be assessed by known techniques, such as Chromium (Cr) release assays or dead cell indicator dyes (propidium Iodide, TO-PRO-3 Iodide).

In some embodiments of a method of inhibiting tumor formation or growth or both, the pre-cancerous stem cells comprise pre-hematological cancer stem cells. In some embodiments of a method of inhibiting tumor formation or growth or both, said tumor cells comprise hematological cancer cells. In some embodiments, hematological tumors are cancer types affecting blood, bone marrow, and lymph nodes. Hematological tumors may derive from either of the two major blood cell lineages: myeloid and lymphoid cell lines. The myeloid cell line normally produces granulocytes, erythrocytes, thrombocytes, macrophages, and masT-cells, whereas the lymphoid cell line produces B, T, and plasma cells. Lymphomas (e.g. Hodgkin's Lymphoma), lymphocytic leukemias, and myeloma are derived from the lymphoid line, while acute and chronic myelogenous leukemia (AML, CML), myelodysplastic syndromes and myeloproliferative diseases are myeloid in origin. In some embodiments of a method of inhibiting tumor formation or growth or both, pre-cancerous stem cells comprise pre-leukemic cancer stem cells. In some embodiments, a hematological cancer comprises leukemia, lymphoma, myeloma, acute myeloid leukemia (AML), acute promyelocytic leukemia, erythroleukemia, biphenotypic B myelomonocytic leukemia, or myelodysplastic syndromes (MDS).

In some embodiments of a method of inhibiting tumor formation or growth or both, the pre-cancerous stem cells comprise a solid cancer or tumor. In some embodiments, the solid cancer or tumor comprises sarcoma, osteosarcoma, squamous cell carcinoma of the head and neck, non-small-cell lung carcinoma, bladder cancer, pancreatic cancer, or pancreatic ductal adenocarcinoma.

In some embodiments of a method of inhibiting tumor formation or growth or both, said subject is a human.

In some embodiments, disclosed herein is a method of treating a subject suffering from a disease, comprising the step of administering to said subject an anti-IL-8 antibody comprising an antibody antigen-binding domain comprising a heavy chain variable region (VH) and a light chain variable region (VL), wherein the disease comprises a cancer or tumor or a viral infection, or a disease associated with inflammation, or a combination thereof, and wherein the amino acid sequences of a VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35, thereby treating said disease in said subject. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 2 and 3.

In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 4 and 5. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 6 and 7. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 8 and 9. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 10 and 11. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 12 and 13. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 14 and 15. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 16 and 17. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 18 and 19. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 20 and 21. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 22 and 23. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 24 and 25. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 26 and 27. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 28 and 29. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 30 and 31. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 32 and 33. In some embodiments, a method of treating a subject suffering from a disease, comprises the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair comprises the paired sequences set forth in SEQ ID NOs: 34 and 35. In some embodiments, a method of treating a subject suffering from a disease, comprises treating a cancer or tumor. In some embodiments, a method of treating a subject suffering from a disease, comprises treating a viral infection. In some embodiments, a method of treating a subject suffering from a disease, comprises treating a disease associated with inflammation, or a combination thereof,

In certain embodiments, disclosed herein is a method of treating a subject suffering from a disease, said method comprising the step of administering to said subject an anti-IL-8 antibody having complementarity determining region (CDR) sequences as set forth in Table 1F, wherein the disease comprises a cancer or tumor or a viral infection, or a disease associated with inflammation, or a combination thereof, and wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, wherein said HCDR1, HCDR2, HCDR3, LCDR1, LCDR2 and LCDR3 for each of said antibody comprises the amino acid sequences as set forth in Table 1F, thereby treating said disease in said subject. In certain embodiments, disclosed herein is a method of treating a subject suffering from a disease, said method comprising the step of administering to said subject an anti-IL-8 antibody having complementarity determining region (CDR) sequences as set forth in Table 1F, wherein the disease comprises a cancer or tumor or a viral infection or a disease associated with inflammation or a combination thereof, and wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, said HCDR1, HCDR2, HCDR3, LCDR1, LCDR2 and LCDR3 for each of said antibody comprises the amino acid sequences as set forth:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109;
   thereby treating said disease in said subject.

In some embodiments, a method of treating disclosed herein reduces the minimal residual disease, increases remission, increases remission duration, reduces tumor relapse rate, prevents metastasis of the tumor or the cancer, or reduces the rate of metastasis of the tumor or the cancer, reduces the severity of the viral infection, improves the immune response to a viral infection, reduces inflammation, or reduces swelling, or any combination thereof, in the treated subject compared with a subject not administered with the anti-IL-8 antibody or a pharmaceutical composition thereof.

A skilled artisan would appreciate that the term “treating” and grammatical forms thereof, may in some embodiments encompass both therapeutic treatment and prophylactic or preventative measures with respect to a tumor or cancer or viral infection, as described herein, wherein the object is to prevent or lessen the targeted tumor or cancer or viral infection as described herein. Thus, in some embodiments of methods disclosed herein, treating may include directly affecting or curing, suppressing, inhibiting, preventing, reducing the severity of, delaying the onset of, reducing symptoms associated with the disease, disorder or condition, or a combination thereof; for example, when said disease or disorder comprises a cancer or tumor or a viral infection or a disease associated with inflammation or a combination thereof. Thus, in some embodiments, “treating” encompasses preventing, delaying progression, inhibiting the growth of, delaying disease progression, reducing tumor load, reducing the incidence of, expediting remission, inducing remission, augmenting remission, speeding recovery, increasing efficacy of or decreasing resistance to alternative therapeutics, reducing the impact of the infection, improving the immune response to the infection, or a combination thereof. In some embodiments, “preventing” encompasses delaying the onset of symptoms, preventing relapse to a disease, decreasing the number or frequency of relapse episodes, increasing latency between symptomatic episodes, preventing the effect of cytokine release syndrome or cytokine storm, reducing cytokine release syndrome or cytokine storm, or a combination thereof. In some embodiments, “suppressing” or “inhibiting”, encompass reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, or a combination thereof.

In some embodiments of a method of treating disclosed herein, the size of a cancer or tumor is reduced. In some embodiments, the growth rate of a cancer or tumor is reduced. In some embodiments, the size or the growth rate or a combination thereof, of a cancer or tumor is reduced. In some embodiments, the negative impact of a viral infection is reduced, for example but not limited to reduction of a cytokine release syndrome or a cytokine storm in an affected tissue or organ. In some embodiments of a method of treating, administration of an anti-IL-8 antibody decreases the time period of a viral infection. In some embodiments of a method of treating, administration of an anti-IL-8 antibody increases clearance of viral infection. In some embodiments, the survival of the subject in need is increased.

A number of diseases and cancer are known to be caused by viruses. Examples of disease-causing viruses include, but are not limited to, norovirus; rotavirus; hepatitis virus A, B, C, D, or E; rabies virus, West Nile virus, enterovirus, echovirus, coxsackievirus, herpes simplex virus (HSV), HSV-2, varicella-zoster virus, mosquito-borne viruses, arbovirus, St. Louis encephalitis virus, California encephalitis virus, lymphocytic choriomeningitis virus, human immunodeficiency virus (HIV), poliovirus, zika virus, rubella virus, cytomegalovirus, human papillomavirus (HPV), enterovirus D68, severe acute respiratory syndrome (SARS) coronavirus, Middle East respiratory syndrome coronavirus, SARS coronavirus 2, Epstein-Barr virus, influenza virus, respiratory syncytial virus, polyoma viruses (such as JC virus, BK virus), Ebola virus, Dengue virus, or any combination thereof. In some embodiments of a method of treating a disease, the disease comprises a viral infection.

In some embodiments of a method of treating a disease, the disease comprises a cancer or tumor. In some embodiments of a method of treating a cancer or tumor, the cancer or tumor comprises a hematological cancer. In some embodiments, a hematological cancer comprises leukemia, lymphoma, myeloma, acute myeloid leukemia (AML), acute promyelocytic leukemia, erythroleukemia, biphenotypic B myelomonocytic leukemia, or myelodysplastic syndromes (MDS).

In some embodiments of a method of treating a cancer or tumor, the cancer or tumor comprises a solid cancer or tumor. In some embodiments of a method of treating a cancer or tumor, the solid cancer or tumor comprises a sarcoma, osteosarcoma, squamous cell carcinoma of the head and neck, non-small-cell lung carcinoma, bladder cancer, pancreatic cancer, or pancreatic ductal adenocarcinoma.

A number of diseases are known to be associated with inflammation, including but not limited to asthma, cancer, chronic inflammatory diseases, atherosclerosis, diabetes, and autoimmune and degenerative diseases, arthritis, and rheumatoid arthritis.

In some embodiments, a method of treating a disease associated with inflammation comprises treating A number of diseases are known to be associated with inflammation, including but not limited to asthma, cancer, chronic inflammatory diseases, atherosclerosis, diabetes, and autoimmune and degenerative diseases, arthritis, or rheumatoid arthritis, or any combination thereof.

In some embodiments of treating a disease associated with inflammation, said treating reduces or eliminates swelling in a joint. In some embodiments, disclosed herein is a method of treating a disease associated with inflammation comprising administration of an IL-8 antibody disclosed herein, wherein said treating reduces or eliminates the swelling in a joint.

In some embodiments, methods of treating a disease associated with inflammation reduces swelling in a moving joint selected from ball and socket, saddle, hinge, condyloid, pivot and gliding joint. In some embodiments, methods of treating a disease associated with inflammation reduces swelling in joints selected from a knee, finger, shoulder, elbow, wrist, ankle, toe, or hip joint, or any combination thereof. In some embodiments, methods of treating a disease associated with inflammation reduces inflammation in a subject in need. In some embodiments, methods of treating a disease associated with inflammation reduces inflammation in a moving joint selected from ball and socket, saddle, hinge, condyloid, pivot and gliding joint.

In some embodiments of a method of treating a disease comprising a cancer, a tumor, or a viral infection, or a disease associated with inflammation, or a combination thereof, the subject is a human.

In some embodiments of a method of treating a disease comprising a cancer, a tumor, or a viral infection, or a disease associated with inflammation, or a combination thereof, administration comprises administering an anti-IL-8 antibody. In some embodiments of a method of treating a disease comprising a cancer, a tumor, or a viral infection, or a disease associated with inflammation, or a combination thereof, administration comprises administering a pharmaceutical composition comprising an anti-IL-8 antibody. In some embodiments of a method of treating a disease comprising a cancer, a tumor, or a viral infection, or a disease associated with inflammation, or a combination thereof, administration comprises administering a polynucleotide encoding an anti-IL-8 antibody. In some embodiments of a method of treating a disease comprising a cancer, a tumor, or a viral infection, or a disease associated with inflammation, or a combination thereof, administration comprises administering a pharmaceutical composition comprising a polynucleotide encoding an anti-IL-8 antibody.

In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a heavy chain variable region (VH) and a light chain variable region (VL), wherein the polynucleotide sequences encoding the VH-VL pair are selected from the paired sequences set forth in SEQ ID NOs: 113 and 114, SEQ ID NOs: 115 and 116, SEQ ID NOs: 117 and 118, SEQ ID NOs: 119 and 120, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 113 and 114. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 115 and 116 In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 117 and 118. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 119 and 120. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 121 and 122. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 123 and 124. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 125 and 126. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 127 and 128. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 129 and 130. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 131 and 132. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 133 and 134. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 135 and 136. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 137 and 138. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 139 and 140. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 141 and 142. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 36 and 37. In some embodiments of a method of treating a disease comprising a cancer, a tumor, or a viral infection, or a disease associated with inflammation, or a combination thereof, administration comprises administering a polynucleotide encoding an anti-IL-8 antibody comprising an antibody antigen-binding domain comprising a VH and a VL, wherein the polynucleotide sequences encoding the VH-VL pair comprise the paired sequences set forth in SEQ ID NOs: 38 and 39.

In certain embodiments, anti-IL-8 antibodies can be administered to a subject directly, or by administering to the subject a polynucleotide sequence encoding the polypeptides, such nucleic acid sequence may be carried by a vector.

The exact amount of the present anti-IL-8 antibodies or compositions thereof required to elicit the desired effects will vary from subject to subject, depending on the species, age, gender, weight, and general condition of the subject, the particular polypeptides, the route of administration, and whether other drugs are included in the regimen. Thus, it is not possible to specify an exact amount for every composition. However, an appropriate amount can be determined by one of ordinary skill in the art using routine experimentation. Dosages can vary, and the polypeptides can be administered in one or more (e.g., two or more, three or more, four or more, or five or more) doses daily, for one or more days. Guidance in selecting appropriate doses for antibodies can be readily found in the literature.

In some embodiments of a method described herein tumor or cancer cells secrete IL-8. In some embodiments of a method described herein, tumor or cancer cells have increased secretion of IL-8 compared with a non-tumor or non-cancer cells of the same cell type. In some embodiments of a method described herein tumor or cancer cells express CXCR1 and/or CXCR2 receptors on their cell surface. In some embodiments of a method described herein tumor or cancer cells express CXCR1 and/or CXCR2 receptors on their cell surface compared to non-tumor or non-cancerous cells of the same cell type.

In some embodiments of a method inhibiting tumor formation or growth or a combination thereof, the anti-IL-8 antibody comprises an IgG, an Fv, an scFv, an Fab, an F(ab′)₂, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody, or a combination thereof. In certain embodiments of a method inhibiting tumor formation or growth or a combination thereof, an anti-IL-8 antibody comprising an IgG subclass is selected from an IgG1, IgG2, IgG3, or an IgG4.

In some embodiments of a method treating a subject suffering from a disease comprising a cancer or tumor or viral infection, the anti-IL-8 antibody comprises an IgG, an Fv, an scFv, an Fab, an F(ab′)₂, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody, or a combination thereof. In certain embodiments of a method inhibiting tumor formation or growth or a combination thereof, an anti-IL-8 antibody comprising an IgG subclass is selected from an IgG1, IgG2, IgG3, or an IgG4.

In some embodiments of methods disclosed herein, administration comprises administering a pharmaceutical composition comprising an anti-IL-8 antibody.

In certain embodiments, a polynucleotide sequence encoding an anti-IL-8 antibody as described herein is used in a method of inhibiting tumor formation or growth or a combination thereof, wherein the polynucleotide sequence encodes an antibody comprising a heavy chain variable region (VH) of the anti-IL-8 antibody and a light chain variable region (VL) of the anti-IL-8 antibody, wherein the amino acid sequence of the VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35. In certain embodiments, a polynucleotide sequence encoding an anti-IL-8 antibody as described herein is used in a method of inhibiting tumor formation or growth or a combination thereof, wherein the polynucleotide encodes a heavy chain variable region (VH) of an anti-IL-8 antibody comprising the complementarity determining regions (HCDR) of said VH as set forth in Table 1F and a light chain variable region (VL) of an anti-IL-8 antibody comprising the complementarity determining regions (LCDR) of said VL as set forth in Table 1F, said heavy chain variable region having heavy chain complementarity determining region (HCDR) 1, HCDR2 and HCDR3, and said light chain variable region having light chain complementarity determining region (LCDR) 1, LCDR2 and LCDR3, wherein said HCDR1, HCDR2, HCDR3, LCDR1, LCDR2 and LCDR3 for each of said antibody comprises the amino acid sequences as set forth:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109.

In certain embodiments, a polynucleotide sequence encoding an anti-IL-8 antibody as described herein is used in a method of treating a disease comprising a cancer or a tumor or a viral infection, wherein the polynucleotide sequence encodes an antibody comprising a heavy chain variable region (VH) of the anti-IL-8 antibody and a light chain variable region (VL) of the anti-IL-8 antibody, wherein the amino acid sequence of the VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35. In certain embodiments, a polynucleotide sequence encoding an anti-IL-8 antibody as described herein is used in a method of treating a disease comprising a cancer or a tumor or a viral infection, wherein the polynucleotide sequence encodes a heavy chain variable region (VH) of an anti-IL-8 antibody comprising the complementarity determining regions (HCDR) of said VH as set forth in Table 1F and a light chain variable region (VL) of an anti-IL-8 antibody comprising the complementarity determining regions (LCDR) of said VL as set forth in Table 1F, said heavy chain variable region having heavy chain complementarity determining region (HCDR) 1, HCDR2 and HCDR3, and said light chain variable region having light chain complementarity determining region (LCDR) 1, LCDR2 and LCDR3, wherein said HCDR1, HCDR2, HCDR3, LCDR1, LCDR2 and LCDR3 for each of said antibody comprises the amino acid sequences as set forth:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109.

In some embodiments of a method disclosed herein, the polynucleotide encoding the anti-IL-8 antibody may encode an IgG, a Fv, a scFv, a Fab, or a F(ab′)2. The IgG can be of the subclass of IgG1, IgG2, IgG3, or IgG4. In some embodiments of a method disclosed herein, the polynucleotide encoding the anti-IL-8 antibody may encode a part of a minibody, a diabody, a triabody, a nanobody, or a single domain antibody.

In one embodiment, the subject is a mammal, e.g., a human suffering from one or more IL-18-associated diseases including cancer or viral infections or inflammation associated diseases, or a combination thereof.

In some embodiments, a method of inhibiting tumor formation or growth or a combination thereof comprises the use of an anti-IL-8 antibody disclosed herein as part of a combination therapy with an additional therapeutic agent. In some embodiments, a method of inhibiting tumor formation or growth or a combination thereof comprises the use of a composition described herein comprising an anti-IL-8 antibody as described herein, in combination with an additional therapeutic agent. In some embodiments, in a method of inhibiting tumor formation or growth or a combination thereof, an additional therapeutic agent comprises a chemotherapeutic agent, an anti-viral agent, an immune modulator, or an additional therapeutic antibody, or any combination thereof.

In some embodiments, a method of inhibiting tumor formation or growth or a combination thereof comprises the use of composition comprising an anti-IL-8 antibody as described herein, and an additional therapeutic agent, comprised in the same composition. In some embodiments, a method of inhibiting tumor formation or growth or a combination thereof comprises the use of an anti-IL-8 antibody as described herein, and an additional therapeutic agent, comprised in different compositions. In some embodiments, a method of inhibiting tumor formation or growth or a combination thereof comprises the use of composition or compositions comprising a combination of an anti-IL-8 antibody as described herein and an additional therapeutic agent, wherein administration is concurrent. In some embodiments, a method of inhibiting tumor formation or growth or a combination thereof comprises the use of an anti-IL-8 antibody as described herein and an additional therapeutic agent, or compositions thereof, wherein administration of an anti-IL-8 antibody or a composition thereof, is prior to administration of the additional therapeutic agent. In some embodiments, a method of inhibiting tumor formation or growth or a combination thereof comprises the use of an anti-IL-8 antibody as described herein and an additional therapeutic agent, or compositions thereof wherein administration of an anti-IL-8 antibody or a composition thereof, is following administration of the additional therapeutic agent.

In some embodiments, a method of treating a subject suffering from a disease comprises the use of an anti-IL-8 antibody disclosed herein as part of a combination therapy with an additional therapeutic agent. In some embodiments, a method of treating a subject suffering from a disease comprises the use of a composition described herein comprising an anti-IL-8 antibody as described herein, in combination with an additional therapeutic agent. In some embodiments, in a method of treating a subject suffering from a disease, an additional therapeutic agent comprises a chemotherapeutic agent, an anti-viral agent, an immune modulator, or an additional therapeutic antibody, or any combination thereof.

In some embodiments, a method of treating a subject suffering from a disease comprises the use of composition comprising an anti-IL-8 antibody as described herein, and an additional therapeutic agent, comprised in the same composition. In some embodiments, a method of treating a subject suffering from a disease comprises the use of an anti-IL-8 antibody as described herein, and an additional therapeutic agent, comprised in different compositions.

In some embodiments, a method of treating a subject suffering from a disease comprises the use of composition or compositions comprising a combination of an anti-IL-8 antibody as described herein and an additional therapeutic agent, wherein administration is concurrent. In some embodiments, a method of treating a subject suffering from a disease comprises the use of an anti-IL-8 antibody as described herein and an additional therapeutic agent, or compositions thereof, wherein administration of an anti-IL-8 antibody or a composition thereof, is prior to administration of the additional therapeutic agent. In some embodiments, a method of treating a subject suffering from a disease comprises the use of an anti-IL-8 antibody as described herein and an additional therapeutic agent, or compositions thereof wherein administration of an anti-IL-8 antibody or a composition thereof, is following administration of the additional therapeutic agent.

The precise dosage and duration of treatment is a function of the disease being treated and may be determined empirically using known testing protocols or by testing the compositions in model systems known in the art and extrapolating therefrom. Controlled clinical trials may also be performed. Dosages may also vary with the severity of the condition to be alleviated. A pharmaceutical composition is generally formulated and administered to exert a therapeutically useful effect while minimizing undesirable side effects. The composition may be administered one time or may be divided into a number of smaller doses to be administered at intervals of time. For any particular subject, specific dosage regimens may be adjusted over time according to the individual need.

As used herein, the singular form “a”, “an” and “the” include plural references unless the context clearly dictates otherwise. For example, the term “an immunoglobulin” or “at least one immunoglobulin” may include a plurality of immunoglobulins, including mixtures thereof.

Throughout this application, various embodiments may be presented in a range format. It should be understood that the description in range format is merely for convenience and brevity and should not be construed as an inflexible limitation on the scope of the invention. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Embodiments of the IL-8 antibodies and uses thereof include the following.

An isolated anti-IL-8 antibody comprising an antibody antigen-binding domain comprising a heavy chain variable region (VH) and a light chain variable region (VL), wherein the amino acid sequences of a VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35.

A composition comprising an isolated anti-IL-8 antibody comprising an antibody antigen-binding domain comprising a heavy chain variable region (VH) and a light chain variable region (VL), wherein the amino acid sequences of a VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35; and a pharmaceutically acceptable carrier.

An isolated anti-IL-8 antibody having complementarity determining region (CDR) sequences as set forth in Table 1F, wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, wherein said HCDR1, HCDR2, HCDR3, LCDR1, LCDR2 and LCDR3 for each of said antibody comprise the amino acid sequences as set forth in Table 1F.

An isolated anti-IL-8 antibody comprising an IgG, an Fv, an scFv, an Fab, an F(ab′)2, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody.

An isolated anti-IL-8 antibody comprising an IgG1, IgG2, IgG3, or an IgG4.

A composition comprising an isolated anti-IL-8 antibody having complementarity determining region (CDR) sequences as set forth in Table 1F, wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, wherein said HCDR1, HCDR2, HCDR3, LCDR1, LCDR2 and LCDR3 for each of said antibody comprise the amino acid sequences as set forth in Table 1F; and a pharmaceutically acceptable carrier.

An isolated polynucleotide sequence encoding a heavy chain variable region (VH) of an anti-IL-8 antibody and a light chain variable region (VL) of the anti-IL-8 antibody, wherein the amino acid sequence of the VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35.

An isolated polynucleotide sequence encoding a heavy chain variable region (VH) of an anti-IL-8 antibody comprising the complementarity determining regions (HCDR) of said VH as set forth in Table 1F and a light chain variable region (VL) of an anti-IL-8 antibody comprising the complementarity determining regions (LCDR) of said VL as set forth in Table 1F, wherein said heavy chain variable region comprises heavy chain complementarity determining region (HCDR) 1, HCDR2 and HCDR3, and said light chain variable region comprises light chain complementarity determining region (LCDR) 1, LCDR2 and LCDR3, wherein said HCDR1, HCDR2, HCDR3, LCDR1, LCDR2 and LCDR3 for each of said VH and CL comprise the amino acid sequences as set forth in Table 1F:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109.

An isolated polynucleotide sequence comprising two polynucleotide sequences, a first polynucleotide sequence encoding the VH of the anti-IL-8 antibody and a second polynucleotide sequence encoding the VL of the anti-IL-8 antibody.

A vector comprising a polynucleotide sequence encoding a heavy chain variable region (VH) of an anti-IL-8 antibody and a light chain variable region (VL) of the anti-IL-8 antibody.

A host cell comprising a vector of claim comprising a polynucleotide sequence encoding a heavy chain variable region (VH) of an anti-IL-8 antibody and a light chain variable region (VL) of the anti-IL-8 antibody.

A method of producing an anti-IL-8 antibody comprising a heavy chain variable region (VH) and a light chain variable region (VH), said method comprises the step of culturing the host cell comprising a vector comprising a polynucleotide sequence encoding a heavy chain variable region (VH) of an anti-IL-8 antibody and a light chain variable region (VL) of the anti-IL-8 antibody, under conditions conducive to expressing said vector in said host cell, and expressing said polynucleotide sequences comprised in said vector, thereby producing the anti-IL-8 antibody comprising a VH and a VL.

A method of inhibiting tumor or cancer formation or growth or a combination thereof in a subject in need, said method comprising the step of administering to said subject an anti-IL-8 antibody comprising an antibody antigen-binding domain comprising a heavy chain variable region (VH) and a light chain variable region (VL), wherein the amino acid sequences of a VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35, thereby inhibiting tumor formation or growth or a combination thereof in said subject.

A method of inhibiting tumor or cancer formation or growth or a combination thereof in a subject in need, said method comprising the step of administering to said subject an anti-IL-8 antibody having complementarity determining region (CDR) sequences as set forth in Table 1F, wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, wherein said HCDR1, HCDR2, HCDR3, LCDR1, LCDR2 and LCDR3 for each of said antibody comprise the amino acid sequences as set forth in Table 1F:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109;
thereby inhibiting tumor formation or growth or a combination thereof in said subject.

A method of inhibiting tumor or cancer formation or growth or a combination thereof in a subject in need comprising the step of administering to said subject an anti-IL-8 antibody, wherein said inhibiting inhibits neutrophil or monocyte activation, or a combination thereof within a tumor microenvironment.

A method of inhibiting tumor or cancer formation or growth or a combination thereof in a subject in need comprising the step of administering to said subject an anti-IL-8 antibody, wherein said inhibiting reduces activation of neutrophils or monocytes, or a combination thereof, within a tumor microenvironment.

A method of inhibiting tumor or cancer formation or growth or a combination thereof in a subject in need comprising the step of administering to said subject an anti-IL-8 antibody, wherein said inhibiting decreases viability of pre-cancerous stem cells or tumor cells. In some embodiments, the pre-cancerous stem cells comprise pre-leukemia stem cells.

A method of inhibiting tumor or cancer formation or growth or a combination thereof in a subject in need comprising the step of administering to said subject an anti-IL-8 antibody, wherein cancer or tumor comprises a hematological cancer.

A method of inhibiting tumor or cancer formation or growth or a combination thereof in a subject in need comprising the step of administering to said subject an anti-IL-8 antibody, wherein the cancer comprised a hematological cancer comprising leukemia, lymphoma, myeloma, acute myeloid leukemia (AML), acute promyelocytic leukemia, erythroleukemia, biphenotypic B myelomonocytic leukemia, or myelodysplastic syndromes (MDS).

A method of inhibiting tumor or cancer formation or growth or a combination thereof in a subject in need comprising the step of administering to said subject an anti-IL-8 antibody, wherein said cancer or tumor comprises a solid cancer or tumor.

A method of inhibiting tumor or cancer formation or growth or a combination thereof in a subject in need comprising the step of administering to said subject an anti-IL-8 antibody, wherein said cancer or tumor comprises a solid cancer or tumor, and wherein the solid cancer or tumor comprises sarcoma, osteosarcoma, squamous cell carcinoma of the head and neck, non-small-cell lung carcinoma, bladder cancer, pancreatic cancer, or pancreatic ductal adenocarcinoma.

A method of inhibiting tumor or cancer formation or growth or a combination thereof in a subject in need comprising the step of administering to said subject an anti-IL-8 antibody, wherein said subject is a human.

A method of inhibiting tumor or cancer formation or growth or a combination thereof in a subject in need comprising the step of administering to said subject an anti-IL-8 antibody, wherein said inhibiting inhibits neutrophil or monocyte activation, or a combination thereof within a tumor microenvironment.

A method of inhibiting tumor or cancer formation or growth or a combination thereof in a subject in need comprising the step of administering to said subject an anti-IL-8 antibody, wherein said inhibiting inhibits activation of neutrophils or monocytes, or a combination thereof, within a tumor microenvironment.

A method of inhibiting tumor or cancer formation or growth or a combination thereof in a subject in need comprising the step of administering to said subject an anti-IL-8 antibody, wherein said inhibiting decreases viability of pre-cancerous stem cells or tumor cells. In some embodiments, the pre-cancerous stem cells comprise pre-leukemic stem cells.

A method of inhibiting tumor or cancer formation or growth or a combination thereof in a subject in need comprising the step of administering to said subject an anti-IL-8 antibody, wherein said subject is a human, and wherein
(a) said inhibiting inhibits neutrophil or monocyte activation, or a combination thereof within a tumor microenvironment; or
(b) said inhibiting inhibits activation of neutrophils or monocytes, or a combination thereof, within a tumor microenvironment; or
(c) said inhibiting decreases viability of pre-cancerous stem cells or tumor cells; or
(d) any combination thereof.

A method of treating a subject suffering from a disease, said method comprising the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the amino acid sequences of a VH-VL pair are selected from the paired sequences set forth in SEQ ID NOs: 2 and 3, SEQ ID NOs: 4 and 5, SEQ ID NOs: 6 and 7, SEQ ID NOs: 8 and 9, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35, thereby treating said disease in said subject.

A method of treating a subject suffering from a disease, said method comprising the step of administering to said subject an anti-IL-8 antibody having complementarity determining region (CDR) sequences as set forth in Table 1F, wherein the disease comprises a cancer or tumor or a viral infection or a disease associated with inflammation or a combination thereof, and wherein each antibody comprises a heavy chain variable region having heavy chain complementarity determining region (HCDR) 1, HCDR2 and HCDR3, and a light chain variable region having light chain complementarity determining region (LCDR) 1, LCDR2 and LCDR3, wherein said HCDR1, HCDR2, HCDR3, LCDR1, LCDR2 and LCDR3 for each of said antibody comprise the amino acid sequences as set forth in Table 1F:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109;
   thereby treating said disease in said subject.

A method of treating a subject suffering from a disease, said method comprising the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, wherein said cancer or tumor comprises a hematological cancer.

A method of treating a subject suffering from a disease, said method comprising the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, wherein said hematological cancer comprises leukemia, lymphoma, myeloma, acute myeloid leukemia (AML), acute promyelocytic leukemia, erythroleukemia, biphenotypic B myelomonocytic leukemia, or myelodysplastic syndromes (MDS).

A method of treating a subject suffering from a disease, said method comprising the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, wherein said cancer or tumor comprises a solid cancer or tumor. In some embodiments, the method treats a solid cancer or tumor comprising a sarcoma, osteosarcoma, squamous cell carcinoma of the head and neck, non-small-cell lung carcinoma, bladder cancer, pancreatic cancer, or pancreatic ductal adenocarcinoma.

A method of treating a subject suffering from a disease, said method comprising the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation, or a combination thereof, wherein said subject is a human.

An isolated polynucleotide sequence encoding a heavy chain variable region (VH) of an anti-IL-8 antibody and a light chain variable region (VL) of the anti-IL-8 antibody, wherein the polynucleotide sequences encoding the VH-VL pair are selected from the paired sequences set forth in SEQ ID NOs: 113 and 114, SEQ ID NOs: 115 and 116, SEQ ID NOs: 117 and 118, SEQ ID NOs: 119 and 120, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39.

An isolated polynucleotide sequence encoding a heavy chain variable region (VH) of an anti-IL-8 antibody and a light chain variable region (VL) of the anti-IL-8 antibody, wherein said polynucleotide sequence comprises two polynucleotide sequences, a first polynucleotide sequence encoding the VH of the anti-IL-8 antibody and a second polynucleotide sequence encoding the VL of the anti-IL-8 antibody.

An isolated polynucleotide sequence encoding a heavy chain variable region (VH) of an anti-IL-8 antibody and a light chain variable region (VL) of the anti-IL-8 antibody, wherein the anti-IL-8 antibody comprises an IgG, an Fv, an scFv, an Fab, an F(ab′)2, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody. In some embodiments, the IgG comprises an IgG1, IgG2, IgG3, or an IgG4.

A vector comprising a polynucleotide sequence encoding a heavy chain variable region (VH) of an anti-IL-8 antibody and a light chain variable region (VL) of the anti-IL-8 antibody, wherein the polynucleotide sequences encoding the VH-VL pair are selected from the paired sequences set forth in SEQ ID NOs: 113 and 114, SEQ ID NOs: 115 and 116, SEQ ID NOs: 117 and 118, SEQ ID NOs: 119 and 120, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39.

A host cell comprising a vector comprising a polynucleotide sequence encoding a heavy chain variable region (VH) of an anti-IL-8 antibody and a light chain variable region (VL) of the anti-IL-8 antibody, wherein the polynucleotide sequences encoding the VH-VL pair are selected from the paired sequences set forth in SEQ ID NOs: 113 and 114, SEQ ID NOs: 115 and 116, SEQ ID NOs: 117 and 118, SEQ ID NOs: 119 and 120, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39.

A method of producing an anti-IL-8 antibody comprising a heavy chain variable region (VH) and a light chain variable region (VH), said method comprises the step of culturing the host cell comprising a vector comprising a polynucleotide sequence encoding a heavy chain variable region (VH) of an anti-IL-8 antibody and a light chain variable region (VL) of the anti-IL-8 antibody, wherein the polynucleotide sequences encoding the VH-VL pair are selected from the paired sequences set forth in SEQ ID NOs: 113 and 114, SEQ ID NOs: 115 and 116, SEQ ID NOs: 117 and 118, SEQ ID NOs: 119 and 120, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39. under conditions conducive to expressing said vector in said host cell, and expressing said polynucleotide sequences comprised in said vector, thereby producing the anti-IL-8 antibody comprising a VH and a VL.

A method of inhibiting tumor or cancer formation or growth or a combination thereof in a subject in need, said method comprising the step of administering to said subject a polynucleotide encoding an anti-IL-8 antibody comprising an antibody antigen-binding domain comprising a heavy chain variable region (VH) and a light chain variable region (VL), wherein the polynucleotide sequences encoding the VH-VL pair are selected from the paired sequences set forth in SEQ ID NOs: 113 and 114, SEQ ID NOs: 115 and 116, SEQ ID NOs: 117 and 118, SEQ ID NOs: 119 and 120, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39, thereby inhibiting tumor formation or growth or a combination thereof in said subject.

A method of inhibiting tumor or cancer formation or growth or a combination thereof in a subject in need, said method comprising the step of administering to said subject a polynucleotide encoding an anti-IL-8 antibody comprising an antibody antigen-binding domain comprising a heavy chain variable region (VH) and a light chain variable region (VL), wherein said inhibiting inhibits neutrophil or monocyte activation, or a combination thereof within a tumor microenvironment.

A method of inhibiting tumor or cancer formation or growth or a combination thereof in a subject in need, said method comprising the step of administering to said subject a polynucleotide encoding an anti-IL-8 antibody comprising an antibody antigen-binding domain comprising a heavy chain variable region (VH) and a light chain variable region (VL), wherein said inhibiting reduces activation of neutrophils or monocytes, or a combination thereof, within a tumor microenvironment.

A method of inhibiting tumor or cancer formation or growth or a combination thereof in a subject in need, said method comprising the step of administering to said subject a polynucleotide encoding an anti-IL-8 antibody comprising an antibody antigen-binding domain comprising a heavy chain variable region (VH) and a light chain variable region (VL), wherein said inhibiting decreases viability of pre-cancerous stem cells or tumor cells.

A method of inhibiting tumor or cancer formation or growth or a combination thereof in a subject in need, said method comprising the step of administering to said subject a polynucleotide encoding an anti-IL-8 antibody comprising an antibody antigen-binding domain comprising a heavy chain variable region (VH) and a light chain variable region (VL), wherein said pre-cancerous stem cells comprise pre-leukemia stem cells.

A method of inhibiting tumor or cancer formation or growth or a combination thereof in a subject in need, said method comprising the step of administering to said subject a polynucleotide encoding an anti-IL-8 antibody comprising an antibody antigen-binding domain comprising a heavy chain variable region (VH) and a light chain variable region (VL), wherein said cancer or tumor comprises a hematological cancer. In some embodiments, the hematological cancer comprises leukemia, lymphoma, myeloma, acute myeloid leukemia (AML), acute promyelocytic leukemia, erythroleukemia, biphenotypic B myelomonocytic leukemia, or myelodysplastic syndromes (MDS).

A method of inhibiting tumor or cancer formation or growth or a combination thereof in a subject in need, said method comprising the step of administering to said subject a polynucleotide encoding an anti-IL-8 antibody comprising an antibody antigen-binding domain comprising a heavy chain variable region (VH) and a light chain variable region (VL), wherein said cancer or tumor comprises a solid cancer or tumor. In some embodiments, the solid cancer or tumor comprises sarcoma, osteosarcoma, squamous cell carcinoma of the head and neck, non-small-cell lung carcinoma, bladder cancer, pancreatic cancer, or pancreatic ductal adenocarcinoma.

A method of inhibiting tumor or cancer formation or growth or a combination thereof in a subject in need, said method comprising the step of administering to said subject a polynucleotide encoding an anti-IL-8 antibody comprising an antibody antigen-binding domain comprising a heavy chain variable region (VH) and a light chain variable region (VL), wherein said subject is a human.

A method of treating a subject suffering from a disease, said method comprising the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation or a combination thereof, and wherein the polynucleotide sequences encoding the VH-VL pair are selected from the paired sequences set forth in SEQ ID NOs: 113 and 114, SEQ ID NOs: 115 and 116, SEQ ID NOs: 117 and 118, SEQ ID NOs: 119 and 120, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39, thereby treating said disease in said subject.

A method of treating a subject suffering from a disease, said method comprising the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation, or a combination thereof, and wherein said cancer or tumor comprises a hematological cancer. In some embodiments, the hematological cancer comprises leukemia, lymphoma, myeloma, acute myeloid leukemia (AML), acute promyelocytic leukemia, erythroleukemia, biphenotypic B myelomonocytic leukemia, or myelodysplastic syndromes (MDS).

A method of treating a subject suffering from a disease, said method comprising the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation, or a combination thereof, and wherein said cancer or tumor comprises a solid cancer or tumor. In some embodiments, the solid cancer or tumor comprises sarcoma, osteosarcoma, squamous cell carcinoma of the head and neck, non-small-cell lung carcinoma, bladder cancer, pancreatic cancer, or pancreatic ductal adenocarcinoma.

A method of treating a subject suffering from a disease, said method comprising the step of administering to said subject an anti-IL-8 antibody comprising an antibody antigen-binding domain comprising a heavy chain variable region (VH) and a light chain variable region (VL), wherein the disease comprises a cancer or tumor or a viral infection or a disease associated with inflammation, or a combination thereof, wherein said subject is a human.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

### EXAMPLES

### Example 1: Materials and Methods

Immunization, Hybridoma Generation and Antibody Recovery:

Recombinant human IL-8 (aa 28-99; SEQ ID NO: 1) (Sino Biological, Cat #10098-HNCH2) was used for immunization following confirmation binding to anti-IL-8 antibody (R&D, Cat #mAb208) by ELISA. A group of 24 Alivamab mice (AMM-KL) (Ablexis transgenic mice—Ablexis, LLC, San Francisco, Calif.) were immunized with human IL-8 following the AMMPD-4 immunization protocol (ADS) and the titers were assessed twice. Additionally, 14 AMM-KL and 3 AMM-LO Alivamab mice were immunized following the AMMPD-2 immunization protocol (ADS) and the titers were assessed once at the end of the process. On day 10 high titers were observed and samples (lymph nodes and spleens) from 41 mice in total were collected and pooled into groups based on immunization strategy, mouse strain and titer results. Samples were processed for B-cell enrichment using magnetic negative selection and hybridomas were generated using electrofusion. Hybridomas were plated in 30×384-well plates at concentrations ranging from 1-3 hybridomas per well and remaining material was cryopreserved. Grown hybridoma colonies were counted on day 7 and saturated supernatants were collected for screening on day 8. Positive hybridomas were expanded to 96 well plates on day 11.

The anti-IL-8 mAb clones used in throughout the Examples below were recombinant IgG1 antibodies comprising the heavy and light chain CDR regions present in positive hybridomas.

Binding Antibodies to IL-8 by ELISA:

Recombinant human or cynomolgus IL-8 (Sino Biological, Cat #10098-HNCH2 and Kingfisher, Cat #RP1321Y-100, respectively) was coated at 1 ug/mL in PBS pH 7.4 on Nunc Maxisorp high-binding black 384-well plates (Thermo Fisher, Cat #460518) overnight at 4° C. After 3× washes with wash buffer (0.05% Tween-20 in 1×PBS, pH 7.5) plates were blocked (1% BSA in PBS) for 1 hour at room temperature. Following, plates were washed 4× and incubated with dilution series of monoclonal antibodies (mAb) (in 0.02% Tween-20 in PBS) for 1 hour at room temperature. The mAb were tested at the highest concentration of 100 nM followed by seven serial half log dilutions. After 4× washes, plates were incubated with anti-human-Fc-HRP detection antibody (Chimerigen, Cat #HF-2208, 1:10,000, in 0.02% Tween-20 in PBS). Following, plates were washed 5×, Supersignal ELISA Pico substrate (Thermofisher, Cat #37069) was added and chemiluminescent signal was read on Sprectramax L for 200 ms/well.

Binding Antibodies to IL-8 by Octet:

mAb were loaded in 3 point dilution series (25 nM, 12.5 nM, 6.25 nM) against either human or cynomolgus IL-8 (Sino Biological, Cat #10098-H01H1 and Kingfisher, Cat #RP1321Y, respectively) onto 16 channel anti-human-Fc capture sensors (Fortebio, Cat #1810172). Human or cynomolgus IL-8 were titrated starting from the highest concentration of 50 nM and followed by 1:1 dilution for 4 or 3 points, respectively. The experimental parameters followed to determine the kinetic constants were Baseline for 60s, Loading (antibody to sensor) for 180 s, Association (analyte to antibody) for 120 s, Dissociation for 1200 s and Regeneration for 4×30 s.

IL-8 Interaction with its Receptors by Flow Cytometry:

mAbs were mixed in serial dilutions with IL-8-Fc (aa28-99 SEQ ID NO: 1, Sino Biological, Cat #10098-H01H). The highest concentration of mAbs tested was 1×10⁻⁷M followed by 9 serial dilutions (1:3.3) and IL-8 was used at the concentration of 10 nM. The mixtures were coated on 96-well plate and incubated for 15 minutes at room temperature. Separately, HEK293-CXCR2 cells were dissociated with cell stripper, filtered through 40 μm and washed 1× with FACS buffer (1% BSA/2 mM EDTA/0.01% NAN3 in PBS pH 7.4). Following, cell suspensions (25 μl) at concentration of 4×10⁶/ml were mixed with each mAb dilution and incubated initially for 15 minutes at room temperature followed by 30 minutes at 4° C. Cells were than washed with FACS buffer and stained with anti-huFc647 (Jackson, Code #109607008, 1:2000) and HelixBlue (1:1000) for 30 min at 4° C. After 2× washes cells were resuspended into FACS buffer (25 μl) and samples were analyzed by flow cytometry using FlowJo software.

IL-8-induced NFkB Reporter Assay:

The cDNA sequences of CXCR1 and CXCR2 (R&D systems, Cat #RDC00025 and Cat #RDC00027, respectively) were cloned into expression vectors (ADS) and constructs were transfected into HEK293 cells. Cells were placed under antibiotic selection (hygromycin, 50 μg/ml) and expression of CXCR1 and CXCR2 was confirmed by flow cytometry (anti-CXCR1 antibody, Biolegend, Cat #320605 and anti-CXCR2 antibody, Biolegend, Cat #149305). Cells recombinantly expressing CXCR1 and CXCR2 were cloned to establish stable cells lines and IL-8 binding was confirmed by flow cytometry. The CXCR1-NFκB reporter cells were generated upon transduction of lentiviral NFκB-dependent luciferase construct (G&P Biosciences, Cat #LTR004) into the stable CXCR1 clone. Cells were placed under antibiotics selection (hygromycin, 50 μg/ml and puromycin, 2.5 μg/ml) and the NFκB reporter system was confirmed to monitor IL-8-dependent luciferase activity by Bright-Glo (Promega, Cat #E2620).

The HEK293-CXCR1-NFκB reporter cells were plated at density of 1×10⁴/well in 1% FBS/OptiMEM (50 μl) in 96-well plate and incubated overnight in 37° C./5% CO₂ humified incubator. Separately, the mAbs were mixed in serial dilutions with IL-8 (aa28-99—SEQ ID NO: 1, Sino Biological, Cat #10098-HNCH2) for 15 minutes at room temperature. The highest concentration of each mAb tested was 1×10⁻⁷M followed by 11 serial dilutions (1:2) and the final concentration of IL-8 in the mixture was 10 nM. The mixtures were added to the cells and the plates were incubated for 5.5 hours in 37° C./5% CO₂ humified incubator. Supernatants from each well (50 μl) were mixed with equal volumes of Bright-Glo and luminescent signal was measured with SpectraMax L.

Detection of CXCR1 and CXCR2 on Cell Surface by Flow Cytometry:

Cell were counted and 1×10⁶ cells were collected in round-bottom polystyrene tubes. Cells were washed with 0.5% BSA in PBS and blocked with FcR blocking reagent (Miltenyi Biotec, Inc, Cat #130-059-901) according to manufacturer's recommendations. Following, cells were stained with anti-CXCR1 or anti-CXCR2 antibody (Biolegend, Cat #320605 and Biolegend, Cat #320714, respectively, 5:100) for 30 minutes at 4° C. in the dark. The expression of CXCR1 and CXCR2 receptor on cell surface was evaluated by flow cytometry on BD Facs Canto II or BD Facs Aria and data were analyzed by FlowJo software.

Detection of CXCR1 and CXCR2 on Cell Surface by Immunohistochemistry:

Cells were seeded on Nunc™ Lab-Tek™ II 8 well chamber Slide™ (ThermoFisher, Cat #154534PK) at concentration of 0.03×10⁶/well (500 l) and incubated overnight in 37° C./5% CO₂ humified incubator. Following, cells were pre-fixed with culture medium (250 l) and equal volume of pre-chilled 4% paraformaldehyde (Fisher Scientific, Cat #AAJ19943K2) for 10 minutes on ice. Cells were then washed 2× with pre-chilled PBS, fixed with pre-chilled 4% paraformaldehyde (250 l) for 10 minutes on ice and washed again 3× with pre-chilled PBS. All samples were analyzed in duplicates following the mouse and rabbit specific HRP/DAB (ABC) detection IHC kit (Abcam, Cat #ab64264). Cells were covered with hydrogen peroxide drops and incubated for 10 minutes at room temperature. After 2× washes (in PBS), protein block drops were applied for 10 minutes at room temperature. Cells were then washed 4× and incubated with anti-CXCR1 or anti-CXCR2 antibodies (Boster Cat #PA2080, 1:500 for CXCR1, Abcam Cat #89251, 1:200 for CXCR1 and ProteinTech Cat #20634-1-AP, 1:200 for CXCR2) in 1% BSA in TBS overnight at 4° C. Following, cells were washed 4× and incubated with biotinylated goat anti-polyvalent for 10 minutes at room temperature. Cells were then washed 4× and incubated with streptavidin peroxidase for 10 minutes at room temperature. The slides were rinsed 4× with PBS followed by incubation with DAB Chromogen for 1-10 minutes. Later, cells were rinsed 4× in PBS and incubated with hematoxylin (Abcam Cat #220365, 1:2 in ddH₂O) for 1-5 minutes. Finally, cells were rinsed with tap water, mounting media was applied on the slide, cover slip was added, and images were observed with Cytation™ Cell Imaging Multi-Mode Reader (BioTek).

IL-8-Induced CXCR2 Internalization:

THP-1 cells were counted and for each condition 1×10⁶ cells were collected in round-bottom polystyrene tubes. Cells were treated with either IL-8 alone (12.5 nM) or IL-8 together with three different concentrations of each mAb (1, 10 or 100 nM) for 30 minutes in 37° C./5% CO₂ humified incubator. After incubation, cells were harvested, washed with 0.5% BSA in PBS and blocked with FcR reagent (Miltenyi Biotec, Inc, Cat #130-059-901) according to manufacturer's recommendations. Following, cells were stained with anti-CXCR2 antibody (Biologend, Cat #320714, 5 μl antibody per million cells per 100 μl) for 30 minutes on ice in the dark. CXCR2 expression was detected by flow cytometry using BD Facs Canto and data were analyzed by FlowJo software.

Antibody Assessment by SDS-PAGE:

mAb samples (2 μg) were mixed with 4× loading buffer and either N-ethyl maleimide (1 μl) for non-reducing conditions or DTT (1 μl of 1M) for reducing conditions. Samples prepared in reducing conditions were boiled at 95° C. for 5 minutes and cooled to 4° C. prior to loading onto a RunBlue SDS Gel 4-20% (Expedeon, Cat #NXG42012). All samples were run using diluted RunBlue 20×SDS running buffer (Expedeon, Cat #NXB50500) at constant 200V for 50 minutes. Following, gels were washed for 1 minute with water and stained with InstantBlue (Expedeon, Cat #ISB1L) for 6 hours. Gels were then detained in water and images were captured with Azure Biosystems c200 on visible light setting.

Antibody Size-Exclusion Analysis:

Size-exclusion analysis was conducted in HPLC (Agilent 1260) equilibrated in isocratic running buffer (0.1M Na₂PO₄ and 0.1M Na₂O₄, pH 6.7). mAb samples (10 μg) were injected with refrigerated autosampler and 40 μl injection loop onto TSKgel SuperSW3000 (TOSOH, Cat #18675) at a flow rate of 0.35 ml/min. Absorbance was measured at 280 nm, 254 nm, and 215 nm via diode array detector and the % main peak was calculated by AUC in Chemstation software.

Detection of IL-8 Secretion Level of Cancer Cell Lines by ELISA:

Cells were seeded on 12-well plates (USA Scientific, Cat #CC7682-7512) at a density of 1×10⁶/well and were cultured in 1 ml media for 72 hours in 37° C./5% CO₂ humified incubator. Culture media was collected from wells, transferred to 1.5 ml microcentrifuge tubes and centrifuged at 10,000 rpm for 5 minutes at room temperature. Supernatants were collected in new 1.5 ml microcentrifuge tubes and immediately analyzed by ELISA or stored in −80° C. freezer. High-binding 96-well plates were coated with capture antibody (1:250 dilution) in PBS overnight at 4° C. Following, plates were washed 1× (0.02% Tween-80 in PBS) and blocked with assay diluent/blocking buffer (BD, Cat #555213) for 30 minutes at room temperature. Plates were washed again 3× and incubated with cell supernatants (undiluted or 1:10 diluted) for 2 hours at room temperature. Standards of IL-8 were used as reference at the highest concentration of 200 μg/ml followed by 2-fold dilution series. Plates were then washed 4× and incubated with primary and detection antibody (both 1:250 dilution) for 1 hour at room temperature. Capture antibody, primary/secondary mix, and IL-8 standards were provided in IL-8-detection kit (BD OptEIA, Cat #555244). Finally, plates were washed 5× and incubated with TMB substrate (Sigma, Cat #T0440, 100 μl/well). After color development, sulfuric acid (50 μl from 0.16M) was added to stop the reaction and plates were read at 450 nm using Cytation5 (Biotek) plate-reader.

Cell Viability Assay of Patient-Derived AML Samples:

Primary patient-derived acute myeloid leukemia (AML) cells were seeded at a density of 20,000 cells/well in 96-well plate on Study Day 0. The enriched media (75 μl) contained StemSpan™ SFEM media (STEMCELLTechnologies, Cat #09650), 2% FBS (heat inactivated), StemSpan™ CC110 (STEMCELL Technologies, Cat #02697) and recombinant human IL3 (R&D Systems, Cat #203/IL-010/CF). Cells were treated on Day 0 with mAbs at concentration of 40 g/ml followed by 5-fold serial dilutions. Plates were kept at 37° C./5% CO₂ in humified incubator and media was not changed during the duration of the assay. On Day 6, plates were removed from incubator and equilibrated to room temperature for up to 30 minutes. Then CellTiter-Glo was added to wells (100 μl) and plates were mixed for 2 minutes on plate rocker, followed by 10 minutes incubation at room temperature. Luminescent signal was recorded using Tecan plate reader.

Cell Line Viability Assay:

Omniscreen cells lines were seeded at density of 4×10³/well (90 μl) in five 96-well plates (Corning, Cat #3340) (plates A, B, C, D, and E) and were incubated overnight at 37° C./5% CO₂ in humidified incubator. At time point 0, culture medium (10 μl) was added to each well of Plate A and equilibrated for 30 minutes at room temperature. Following, CellTiter-Glo reagent (Promega, Cat #G7572, 50l), was added to each well, mixed for 5 minutes on orbital shaker and incubated for 20 minutes at room temperature. Luminescence was measured using Envision Multi Label Reader (Perkin Elmer, Equip ID: TAREA0020). Cells on Plates B, C and D were treated in triplicates with antibodies (10 μl) at concentration of 200 μg/ml followed by 5-fold serial dilutions to achieve 9 testing concentrations. Cells on Plate E were treated with reference controls. After 72 hours incubation at 37° C./5% CO₂ in a humidified incubator, CellTiter-Glo Reagent was added and plates were read as described for the first time point.

### Example 2: Characterization of Anti-IL-8 mAb Clones

Objective: To examine the physical properties of the recombinant anti-IL-8 mAbs generated, and to characterize the mAb clones.

Methods: mAbs were generated as described in Example 1. Briefly, antibodies were generated following immunization of the AlivaMab mouse, hybridoma fusion, and screens to select for high affinity functional antibodies. Recombinant human IgG1 antibodies were generated, having either human kappa or lambda light chains. Methods to analyze the physical properties of the recombinant anti-IL-8 antibodies were those well known in the field including gel migration under non-reducing and reducing conditions, and HPLC size-exclusion analysis. The recombinant antibody clones used throughout these Examples are fully human IgG1 antibodies, wherein the CDR domains of the monoclonal antibodies are those generated in the Alivamab mice.

The variable heavy chain regions (VH) and variable light chain regions (VL) were sequenced using techniques well known in the field, and nucleotide sequences generated to encode the VH and VL amino sequences of the anti-IL-8 clones.

Results: Illustrative amino acid sequences of variable heavy chain domain (VH) and variable light chain domain (VL) pair are provided in Table 1A below.

TABLE 1A
Variable Heavy chain (VH) domain and Variable Light chain (VL)
domain amino acid sequences of Anti-IL-8 Antibody Clone

| | SEQ | |
|---|---|---|
| Clone | ID | |
| VH/VL | NO: | Amino Acid Sequence |
| STLX3 | 2 | EVQLVQSGAEVKKAGESLKISCKGSGYSFTSYWIGWVRQMP |
| VH | | GKGLEWMGIIYPGDSDTRYSPSFQGQVTISADKSISTAYLQW |
| | | SSLKASDTAMYYCARDITGNFDYWGQGTLVTVSS |
| STLX3 | 3 | DIQMTQSPSSLSASVGDRVTITCQASQDISNYLNWYQQKPGK |
| VL | | APKVLIYDASNLETGVPSRFSGSGSGTDFTFTISSLQPEDIATY |
| | | YCQQYDNFPQLTFGGGTKVEIK |
| STLX5 | 4 | QVQLQESGPGLVKPSETLSLTCTVSGGPISSYYWSWIRQPPG |
| VH | | KGLEWIGYIYYIGSTDYNPSLKSRVTISKDTSKNQFSLKLSS |
| | | VTAADTAVYYCARWELHAFDIWGQGTMVTVSS |
| STLX5 | 5 | DIQMTQSPSSLSASVGDRVTITCQASQDISNYLNWYQQKPGK |
| VL | | APKLLIYDASNLETGVPSRFSGSGSGTDFTFTISSLQPEDIATY |
| | | YCQQYDNLFTFGPGTKVDIK |
| STLX14 | 6 | EVQLVESGGGLVQPGGSLRLSCAASGFTFSSYDMDWVRQAT |
| VH | | GKGLEWVSTIGTAGDTYYPGSVKGRFTISRENAKNSLYLQMN |
| | | SLRAGDTAVYYCARGRAPHWYFDLWGRGTLVTVSS |
| STLX14 | 7 | SYVLTQPPSVSVAPGQTARITCGGNNIGSKSVHWYQQKPGQA |
| VL | | PVLVVYDDSDRPSGIPERFSGSNSGNTATLTISRVEAGDEADY |
| | | YCQVWDSSSDVVFGGGTKLTVL |
| STLX18 | 8 | EVQLVESGGGLVQPGGSLRLSCAASGFTFSSYDMNWVRQAT |
| VH | | GKGLEWVSAIGPAGDTYYPGSVKGRFTISRENAKNSLYLQMNSLR |
| | | AGDTAVYYCARERWPGYFDLWGRGTLVTVSS |
| STLX18 | 9 | SYVLTQPPSVSVAPGQTARITCGGNNIGSKSVHWYQQKPGQA |
| VL | | PVLVVYDDSDRPSGIPERFSGSNSGNTATLTISRVEAGDEADY |
| | | YCQVWDSSSDHVVFGGGTKLTVL |
| STLX21 | 10 | EVQLVQSGAEVKKAGESLKISCKGSGYSFTSYWIGWVRQMPGKGL |
| VH | | EWMGIIYPGSSDTRYSPSFQGQVTISADKSISTAYLQWSSLKASDTA |
| | | MYYCARDITGNFDYWGQGTLVTVSS |
| STLX21 | 11 | DIQMTQSPSSLSASVGDRVTITCQASQDISNYLNWYQQKPGKAPKV |
| VL | | LIYDASNLETGVPSRFSGSGSGTDFTFTISSLQPEDIATYYCQQYDNF |
| | | PQLTFGGGTKVEIK |
| STLX22 | 12 | EVQLVQSGAEVKKAGESLKISCKGSGYSFTSYWIGWVRQMPGKGL |
| VH | | EWMGIIYPGTSDTRYSPSFQGQVTISADKSISTAYLQWSSLKASDTA |
| | | MYYCARDITGNFDYWGQGTLVTVSS |
| STLX22 | 13 | DIQMTQSPSSLSASVGDRVTITCQASQDISNYLNWYQQKPGKAPKV |
| VL | | LIYDASNLETGVPSRFSGSGSGTDFTFTISSLQPEDIATYYCQQYDNF |
| | | PQLTFGGGTKVEIK |
| STLX31 | 14 | EVQLVESGGGLVKPGGSLRLSCAASGFTFSSFSMSWVRQAPGKGLE |
| VH | | WVSSISSSSSYIDYADSVKGRFTISRDNAKNSLYLQMNSLTAEDTAV |
| | | YFCARDVGPYWYFDLWGRGTLVTVSS |
| STLX31 | 15 | SYVLTQPPSVSVAPGQTARITCGGNNIGSKSVHWYQQKPGQAPVLV |
| VL | | VYDESDRPSGILEGFSGSNLGNTATLTISRVEAGDEADYYCQVWDS |
| | | SSDHVVFGGGTKLTVL |
| STLX35 | 16 | QVQLVESGGGEVQPGRSLRLSCPASGFTFSSYGMHWVRQAPGKGL |
| VH | | EWVALISYDGGNKYYADSVKGRFTISRDNSKNTLYLQMNSLRPED |
| | | TAVYYCARDRVGILDYWGQGTLVTVSS |
| STLX35 | 17 | DIQMTQSPSSLSASVGDRVTITCQASQDISNYLNWFQQKPGKAPKLL |
| VL | | IYGASNLETGVPSRFSGSGSGTDFTFTISSLQPEDIATYYCQQYDTLT |
| | | FGGGTKVEIK |
| STLX38 | 18 | QVQLVQSGTEVKKPGASVKVSCKASGYTFTNYYMHWVRQAPGQG |
| VH | | LEWMGVINPSGGGTNYAQKFQGRVTMTRDTSTSTVYMELSSLRSE |
| | | DTAVYYCARDKAPYYGMDVWGQGTTVTVSS |
| STLX38 | 19 | SYVLTQPPSVSVAPGQTARITCGGNKIGSKSVHWYQQKPGQAPVLV |
| VL | | VYEDSDRPSGIPERFSGSNSGNTAALTISRVEAGDEADYYCQVWDS |
| | | SSDHVVFGGGTKLTVL |
| STLX41 | 20 | EVQLVESGGGLVQPGGSLRLSCAASGFTFSSYDMNWVRQATGKGL |
| VH | | EWVSAIGPAGDTYYPGSVKGRFTISRENAKNSLYLQMNSLRAGDT |
| | | AVYYCARERWPGYFDLWGRGTLVTVSS |
| STLX41 | 21 | SYVLTQPPSVSVAPGQTARITCGGNNIGSKSVHWYQQKPGQAPVLV |
| VL | | VYDTSDRPSGIPERFSGSNSGNTATLTISRVEAGDEADYYCQVWDSS |
| | | SDHVVFGGGTKLTVL |
| STLX42 | 22 | EVQLVESGGGLVQPGGSLRLSCAASGFTFSSYDMNWVRQATGKGL |
| VH | | EWVSAIGPAGDTYYPGSVKGRFTISRENAKNSLYLQMNSLRAGDT |
| | | AVYYCARERWPGYFDLWGRGTLVTVSS |
| STLX42 | 23 | SYVLTQPPSVSVAPGQTARITCGGNNIGSKSVHWYQQKPGQAPVLV |
| VL | | VYDDNDRPSGIPERFSGSNSGNTATLTISRVEAGDEADYYCQVWDS |
| | | SSDHVVFGGGTKLTVL |
| STLX43 | 24 | EVQLVESGGGLVQPGGSLRLSCAASGFTFSSYDMNWVRQATGKGL |
| VH | | EWVSAIGPAGDTYYPGSVKGRFTISRENAKNSLYLQMNSLRAGDT |
| | | AVYYCARERWPGYFDLWGRGTLVTVSS |
| STLX43 | 25 | SYVLTQPPSVSVAPGQTARITCGGNNIGSKSVHWYQQKPGQAPVLV |
| VL | | VYDDSDRPSGIPERFSGSNSGNTATLTISRVEAGDEADYYCQVWESS |
| | | SDHVVFGGGTKLTVL |
| STLX44 | 26 | EVQLVESGGGLVQPGGSLRLSCAASGFTFSSYDMNWVRQATGKGL |
| VH | | EWVSAIGPAGDTYYPGSVKGRFTISRENAKNSLYLQMNSLRAGDT |
| | | AVYYCARERWPGYFDLWGRGTLVTVSS |
| STLX44 | 27 | SYVLTQPPSVSVAPGQTARITCGGNNIGSKSVHWYQQKPGQAPVLV |
| VL | | VYDDSDRPSGIPERFSGSNSGNTATLTISRVEAGDEADYYCQVWTSS |
| | | SDHVVFGGGTKLTVL |
| STLX45 | 28 | EVQLVESGGGLVQPGGSLRLSCAASGFTFSSYDMNWVRQATGKGL |
| VH | | EWVSAIGPAGDTYYPGSVKGRFTISRENAKNSLYLQMNSLRAGDT |
| | | AVYYCARERWPGYFDLWGRGTLVTVSS |
| STLX45 | 29 | SYVLTQPPSVSVAPGQTARITCGGNNIGSKSVHWYQQKPGQAPVLV |
| VL | | VYDTSDRPSGIPERFSGSNSGNTATLTISRVEAGDEADYYCQVWESS |
| | | SDHVVFGGGTKLTVL |
| STLX46 | 30 | EVQLVESGGGLVQPGGSLRLSCAASGFTFSSYDMNWVRQATGKGL |
| VH | | EWVSAIGPAGDTYYPGSVKGRFTISRENAKNSLYLQMNSLRAGDT |
| | | AVYYCARERWPGYFDLWGRGTLVTVSS |
| STLX46 | 31 | SYVLTQPPSVSVAPGQTARITCGGNNIGSKSVHWYQQKPGQAPVLV |
| VL | | VYDTSDRPSGIPERFSGSNSGNTATLTISRVEAGDEADYYCQVWTSS |
| | | SDHVVFGGGTKLTVL |
| STLX47 | 32 | EVQLVESGGGLVQPGGSLRLSCAASGFTFSSYDMNWVRQATGKGL |
| VH | | EWVSAIGPAGDTYYPGSVKGRFTISRENAKNSLYLQMNSLRAGDT |
| | | AVYYCARERWPGYFDLWGRGTLVTVSS |
| STLX47 | 33 | SYVLTQPPSVSVAPGQTARITCGGNNIGSKSVHWYQQKPGQAPVLV |
| VL | | VYDDNDRPSGIPERFSGSNSGNTATLTISRVEAGDEADYYCQVWES |
| | | SSDHVVFGGGTKLTVL |
| STLX48 | 34 | EVQLVESGGGLVQPGGSLRLSCAASGFTFSSYDMNWVRQATGKGL |
| VH | | EWVSAIGPAGDTYYPGSVKGRFTISRENAKNSLYLQMNSLRAGDT |
| | | AVYYCARERWPGYFDLWGRGTLVTVSS |
| STLX48 | 35 | SYVLTQPPSVSVAPGQTARITCGGNNIGSKSVHWYQQKPGQAPVLV |
| VL | | VYDDNDRPSGIPERFSGSNSGNTATLTISRVEAGDEADYYCQVWTS |
| | | SSDHVVFGGGTKLTVL |

Complementarity-determining regions (CDR) for each of the recombinant mAb clones were calculated using two different methods. Tables 1B and 1C present Modeled CDR determinations according to Kabat with some minor modification based on their internal modeling (Kabat, E. A. et al., In: Sequences of Proteins of Immunological Interest, NIH Publication, 91-3242 (1991)). (Table 1B (light chain CDRs) and Table 1C (heavy chain CDRs)). Tables 1D and 1E present Modeled CDR determinations according to the information found at the Immuno Gene Tics website (http://www.imgt.org/IMGTScientificChart/Numbering/IMGTnumbering.html (Table 1D (light chain CDRs) and Table 1E (heavy chain CDRs)).

TABLE 1B
Anti-IL-8 mAb: Kabat Modeled CDR Determinations Variable Light
Chain CDR Amino Acid Sequences

| | | | SEQ | | SEQ | | SEQ |
|---|---|---|---|---|---|---|---|
| | | | ID | | ID | | ID |
| IgG | LC | CDR-L1 | NO | CDR-L2 | NO | CDR-L3 | NO |
| STLX3 | Kappa | QASQDISNYLN | 40 | DASNLET | 43 | QQYDNFPQLT | 50 |
| STLX5 | Kappa | QASQDISNYLN | 40 | DASNLET | 43 | QQYDNLFT | 51 |
| STLX14 | Lambda | GGNNIGSKSVH | 41 | DDSDRPS | 44 | QVWDSSSDVV | 52 |
| STLX18 | Lasabda | GGNNIGSKSVH | 41 | DDSDRPS | 44 | QVWDSSSDHVV | 53 |
| STLX21 | Kappa | QASQDISNYLN | 40 | DASNLET | 43 | QQYDNFPQLT | 50 |
| STLX22 | Kappa | QASQDISNYLN | 40 | DASNLET | 43 | QQYDNFPQLT | 50 |
| STLX31 | Lambda | GGNNIGSKSVH | 41 | DESDRPS | 45 | QVWDSSSDHVV | 53 |
| STLX35 | Kappa | QASQDISNYLN | 40 | GASNLET | 46 | QQYDTLT | 54 |
| STLX38 | Lambda | GGNKIGSKSVH | 42 | EDSDRPS | 47 | QVWDSSSDHVV | 53 |
| STLX41 | Lambda | GGNNIGSKSVH | 41 | DTSDRPS | 48 | QVWDSSSDHVV | 53 |
| STLX42 | Lambda | GGNNIGSKSVH | 41 | DDNDRPS | 49 | QVWDSSSDHVV | 53 |
| STLX43 | Lambda | GGNNIGSKSVH | 41 | DDSDRPS | 44 | QVWESSSDHVV | 55 |
| STLX44 | Lambda | GGNNIGSKSVH | 41 | DDSDRPS | 44 | QVWTSSSDHVV | 56 |
| STLX45 | Lambda | GGNNIGSKSVH | 41 | DTSDRPS | 48 | QVWESSSDHVV | 55 |
| STLX46 | Lambda | GGNNIGSKSVH | 41 | DTSDRPS | 48 | QVWTSSSDHVV | 56 |
| STLX47 | Lambda | GGNNIGSKSVH | 41 | DDNDRPS | 49 | QVWESSSDHVV | 55 |
| STLX48 | Lambda | GGNNIGSKSVH | 41 | DDNDRPS | 49 | QVWTSSSDHVV | 56 |

TABLE 1C
Anti-IL-8 mAb: Kabat Modeled CDR Determinations Variable
Heavy Chain CDR Amino Acid Sequences

| | | SEQ | | SEQ | | SEQ |
|---|---|---|---|---|---|---|
| | | ID | | ID | | ID |
| IgG | CDR-H1 | NO | CDR-H2 | NO | CDR-H3 | NO |
| STLX3 | SYWIG | 57 | IIYPGDSDTRYSPSPQG | 64 | DITGNEDY | 73 |
| STLX5 | SYYWS | 58 | YIYYIGSTDYNPSLKS | 65 | WELHAFDI | 74 |
| STLX14 | SYDMD | 59 | TIGTAGDTYYPGSVKG | 66 | GRAPHWYFDL | 75 |
| STLX18 | SYDMN | 60 | AIGPAGDTYYPGSVKG | 67 | ERWPGYFDL | 76 |
| STLX21 | SYWIG | 57 | IIYPGSSDTRYSPSFQG | 68 | DITGNFDY | 73 |
| STLX22 | SYWIG | 57 | IIYPGTSDTRYSPSFQG | 69 | DITGNFDY | 73 |
| STLX31 | SFSMS | 61 | SISSSSSYIDYADSVKG | 70 | DVGPYWYEDL | 77 |
| STLX35 | SYGMH | 62 | LISYDGGNKYYADSVKG | 71 | DRVGILDY | 78 |
| STLX38 | NYYMH | 63 | VINPSGGGINYAQKFQG | 72 | DKAPYYGMDV | 79 |
| STLX41 | SYDMN | 60 | AIGPAGDTYYPGSVKG | 67 | ERWPGYFDL | 76 |
| STLX42 | SYDMN | 60 | AIGPAGDTYYPGSVKG | 67 | ERWPGYFDL | 76 |
| STLX43 | SYDMN | 60 | AIGPAGDTYYPGSVKG | 67 | ERWPGYFDL | 76 |
| STLX44 | SYDMN | 60 | AIGPAGDTYYPGSVKG | 67 | ERWPGYFDL | 76 |
| STLX45 | SYDMN | 60 | AIGPAGDTYYPGSVKG | 67 | ERWPGYFDL | 76 |
| STLX46 | SYDMN | 60 | AIGPAGDTYYPGSVKG | 67 | ERWPGYFDL | 76 |
| STLX47 | SYDMN | 60 | AIGPAGDTYYPGSVKG | 67 | ERWPGYFDL | 76 |
| STLX48 | SYDMN | 60 | AIGPAGDTYYPGSVKG | 67 | ERWPGYFDL | 76 |

TABLE 1D
Anti-IL-8 mAb: IMGT Modeled CDR Determinations Variable
Light Chain CDR Amino Acid Sequences

| | | | SEQ | | SEQ | | SEQ |
|---|---|---|---|---|---|---|---|
| | | | ID | | ID | | ID |
| IgG | LC | CDR-L1 | NO | CDR-L2 | NO | CDR-L3 | NO |
| STLX3 | Kappa | QDISNY | 80 | DAS | 83 | QQYDNFPQLT | 50 |
| STLX5 | Kappa | QDISNY | 80 | DAS | 83 | QQYDNLFT | 51 |
| STLX14 | Lambda | NIGSKS | 81 | DDS | 84 | QVWDSSSDVV | 52 |
| STLX18 | Lambda | NIGSKS | 81 | DDS | 84 | QVWDSSSDHVV | 53 |
| STLX21 | Kappa | QDISNY | 80 | DAS | 83 | QQYDNFPQLT | 50 |
| STLX22 | Kappa | QDISNY | 80 | DAS | 83 | QQYDNFPQLT | 50 |
| STLX31 | Lambda | NIGSKS | 81 | DES | 65 | QVWDSSSDHVV | 53 |
| STLX35 | Kappa | QDISNY | 80 | GAS | 86 | QQYDTLT | 54 |
| STLX38 | Lambda | KIGSKS | 82 | EDS | 87 | QVWDSSSDHVV | 53 |
| STLX41 | Lambda | NIGSKS | 81 | DTS | 88 | QVWDSSSDHVV | 53 |
| STLX42 | Lambda | NIGSKS | 81 | DON | 89 | QVWDSSSDHVV | 53 |
| STLX43 | Lambda | NIGSKS | 81 | DDS | 84 | QVWESSSDHVV | 54 |
| STLX44 | Lambda | NIGSKS | 81 | DDS | 84 | QVWTSSSDHVV | 56 |
| STLX45 | Lambda | NIGSKS | 81 | DTS | 84 | QVWESSSDHVV | 55 |
| STLX46 | Lambda | NIGSKS | B1 | DTS | 88 | QVWTSSSDHVV | 56 |
| STLX47 | Lambda | NIGSKS | 81 | DDN | 89 | QVWESSSDHVV | 55 |
| STLX48 | Lambda | NIGSKS | 81 | DDN | 89 | QVWTSSSDHVV | 56 |

TABLE 1E
Anti-IL-8 mAb: IMGT Modeled CDR Determinations
Variable Heavy Chain CDR Amino Acid Sequences

| | | SEQ | | SEQ | | SEQ |
|---|---|---|---|---|---|---|
| | | ID | | ID | | ID |
| IgG | CDR-H1 | NO | CDR-H2 | NO | CDR-H3 | NO |
| STLX3 | GYSFTSYW | 90 | IYPGDSDT | 97 | ARDITGNEDY | 106 |
| STLX3 | GGPISSYY | 91 | IYYIGST | 98 | ARWELHAFDI | 107 |
| STLX14 | GFTFSSYD | 92 | IGTAGDT | 99 | ARGRAPHWYFDL | 108 |
| STLX18 | GFTFSSYD | 93 | IGPAGDT | 100 | ARERWPGYFDL | 109 |
| STLX21 | GYSFTSYW | 90 | IYPGSSDT | 101 | ARDITGNFDY | 106 |
| STLX22 | GYSPISYW | 90 | IYPGTSDT | 102 | ARDITGNFDY | 106 |
| STLX31 | GFTFSSFS | 94 | ISSSSSYI | 103 | ARDVGPYWYFDL | 118 |
| STLX35 | GFTFSSYG | 95 | ISYDGGNK | 104 | ARDRVGILDY | 111 |
| STLX38 | GYTFINYY | 96 | INPSGGGT | 105 | ARDKAPYYGMDV | 112 |
| STLX41 | GFTFSSYD | 93 | IGPAGDT | 100 | ARERWPGYFDL | 109 |
| STLX42 | GFTFSSYD | 93 | IGPAGDT | 100 | ARERWPGYFDL | 109 |
| STLX43 | GFTFSSYD | 93 | IGPAGDT | 100 | ARERWPGYFDL | 109 |
| STLX44 | GFTFSSYD | 93 | IGPAGDT | 100 | ARERWPGYFDL | 109 |
| STLX45 | GFTFSSYD | 93 | IGPAGDT | 100 | ARERWPGYEDL | 109 |
| STLX46 | GFTFSSYD | 93 | IGPAGDT | 100 | ARERWPGYFDL | 109 |
| STLX47 | GFTFSSYD | 93 | IGPAGDT | 100 | ARERWPGYFDL | 109 |
| STLX48 | GFTFSSYD | 93 | IGPAGDT | 100 | ARERWPGYFDL | 109 |

The light chains of an antibody can be classified as either kappa (κ) or lambda (λ) type. Tables 1B and 1D identify for each clone whether the light chain is a kappa or lambda type. The set of six CDR regions present in each recombinant mAb may be determined based on the CDR sequences provided in Tables 1B and 1C or Tables 1D and 1E, and is provided here in Table 1F below.

TABLE 1F
Amino Acid Sequences of CDR Regions for each Anti-IL-8 mAb.

| | LCDR1 | LCDR2 | LCDR3 | HCDR1 | HCDR2 | HCDR3 |
|---|---|---|---|---|---|---|
| | SEQ ID | SEQ ID | SEQ ID | SEQ ID | SEQ ID | SEQ ID |
| | NO: | NO: | NO: | NO: | NO: | NO: |
| STLX18 | 41 | 44 | 53 | 60 | 67 | 76 |
| STLX18 | 81 | 84 | 53 | 93 | 100 | 109 |
| STLX3 | 40 | 43 | 50 | 57 | 64 | 73 |
| STLX5 | 40 | 43 | 51 | 58 | 65 | 74 |
| STLX14 | 41 | 44 | 52 | 59 | 66 | 75 |
| STLX21 | 40 | 43 | 50 | 57 | 68 | 73 |
| STLX22 | 40 | 43 | 50 | 57 | 69 | 73 |
| STLX31 | 41 | 45 | 53 | 61 | 70 | 77 |
| STLX35 | 40 | 46 | 54 | 62 | 71 | 78 |
| STLX38 | 42 | 47 | 53 | 63 | 72 | 79 |
| STLX41 | 41 | 48 | 53 | 60 | 67 | 76 |
| STLX42 | 41 | 49 | 53 | 60 | 67 | 76 |
| STLX43 | 41 | 44 | 55 | 60 | 67 | 76 |
| STLX44 | 41 | 44 | 56 | 60 | 67 | 76 |
| STLX45 | 41 | 48 | 55 | 60 | 67 | 76 |
| STLX46 | 41 | 48 | 56 | 60 | 67 | 76 |
| STLX47 | 41 | 49 | 55 | 60 | 67 | 76 |
| STLX48 | 41 | 49 | 56 | 60 | 67 | 76 |
| STLX3 | 80 | 83 | 50 | 90 | 97 | 106 |
| STLX5 | 80 | 83 | 51 | 91 | 98 | 107 |
| STLX14 | 81 | 84 | 52 | 92 | 99 | 108 |
| STLX21 | 80 | 83 | 50 | 90 | 101 | 106 |
| STLX22 | 80 | 83 | 50 | 90 | 102 | 106 |
| STLX31 | 81 | 85 | 53 | 94 | 103 | 110 |
| STLX35 | 80 | 86 | 54 | 95 | 104 | 111 |
| STLX38 | 82 | 87 | 53 | 96 | 105 | 112 |
| STLX41 | 81 | 88 | 53 | 93 | 100 | 109 |
| STLX42 | 81 | 89 | 53 | 93 | 100 | 109 |
| STLX43 | 81 | 84 | 55 | 93 | 100 | 109 |
| STLX44 | 81 | 84 | 56 | 93 | 100 | 109 |
| STLX45 | 81 | 88 | 55 | 93 | 100 | 109 |
| STLX46 | 81 | 88 | 56 | 93 | 100 | 109 |
| STLX47 | 81 | 89 | 55 | 93 | 100 | 109 |
| STLX48 | 81 | 89 | 56 | 93 | 100 | 109 |

TABLE 1G
Nucleotide Sequences encoding the Variable
Heavy chain (VH) domain and Variable Light
chain (VL) domain of Anti-IL-8 Antibody Clones.

| | SEQ | |
|---|---|---|
| Clone | ID | |
| VH/VL | NO: | Nucleotide Sequence |
| STLX3 VH | 113 | gaagttcagctggttcagtctggcg |
| | | ccgaagtgaagaaggctggcgagtc |
| | | cctgaagatctcctgcaaaggctcc |
| | | ggctactccttcacctcctactgga |
| | | tcggctgggtccgacagatgcctgg |
| | | caaaggcttggagtggatgggcatc |
| | | atctaccccggcgactctgatacca |
| | | gatactcccctagcttccagggcca |
| | | agtgaccatctccgccgacaagtct |
| | | atctccaccgcctacctgcagtggt |
| | | cctctctgaaggcttctgacaccgc |
| | | catgtactactgcgccagagacatc |
| | | accggcaacttcgactattggggcc |
| | | agggcacactggtcaccgtgtcctc |
| | | t |
| STLX3 VL | 114 | gacatccagatgacccagtctccat |
| | | cctctctgtccgcctctgtgggcga |
| | | cagagtgaccattacctgtcaggcc |
| | | agccaggacatctccaactacct |
| | | gaactggtatcagcagaagcccggc |
| | | aaggcccctaaggtgctgatctacg |
| | | atgcctccaacctggaaaccggcgt |
| | | gccctctagattctccggctctggc |
| | | tctggcaccgactttacctttacaa |
| | | tctccagcctgcagcctgaggatat |
| | | cgccacctactactgccagcagtac |
| | | gacaacttccctcagctgacctttg |
| | | gcggaggcaccaaggtggaaatcaa |
| | | g |
| STLX5 VH | 115 | caggttcagctgcaagagtctggcc |
| | | ctggcctggtcaagccttccgaaac |
| | | actgtctctgacctgcaccgtgtct |
| | | ggcggccctatctcctcctactact |
| | | ggtcctggatcagacagcctcctgg |
| | | caaaggcctggaatggatcggctac |
| | | atctactacatcggctccaccgact |
| | | acaaccccagcctgaagtccagagt |
| | | gaccatctccaaggacacctccaag |
| | | aaccagttctccctgaagctgtcct |
| | | ccgtgaccgctgctgataccgccgt |
| | | gtactactgtgccagatgggagctg |
| | | cacgccttcgatatctggggccaggg |
| | | cacaatggtcaccgtgtcctct |
| STLX5 VL | 116 | gacatccagatgacccagtctccat |
| | | cctctctgtccgcctctgtgggcga |
| | | cagagtgaccattacctgtcaggcc |
| | | agccaggacatctccaactacct |
| | | gaactggtatcagcagaagcccggc |
| | | aaggcccctaagctgctgatctacg |
| | | atgcctccaacctggaaaccggcgt |
| | | gccctctagattctccggctctggc |
| | | tctggcaccgactttacctttacaa |
| | | tctccagcctgcagcctgaggatat |
| | | cgccacctactactgccagcagtac |
| | | gacaacctgttcaccttcggacccg |
| | | gcaccaaggtggacatcaaa |
| STLX14 VH | 117 | gaggtgcagctggtggagtctgggg |
| | | gaggcttggtacagcctggggggtc |
| | | cctgagactctcctgtgcagcctct |
| | | ggattcaccttcagtagctacgaca |
| | | tggactgggtccgccaagctacagg |
| | | aaaaggtctggagtgggtctcaact |
| | | attggtactgctggtgacacatact |
| | | atccaggctccgtgaagggccgatt |
| | | caccatctccagagaaaatgccaag |
| | | aactccttgtatcttcaaatgaaca |
| | | gcctgagagccggggacacggctgt |
| | | gtattactgtgcaagagggagggct |
| | | cctcactggtacttcgatctctggg |
| | | gccgtggcaccctggtcactgtctc |
| | | ctca |
| STLX14 VL | 118 | tcctatgtgctgactcagccaccct |
| | | cggtgtcagtggccccaggacagac |
| | | ggccaggattacctgtgggggaaac |
| | | aacattggaagtaaaagtgtg |
| | | cactggtaccagcagaagccaggcc |
| | | aggcccctgtgctggtcgtctatga |
| | | tgatagcgaccggccctcagggatc |
| | | cctgagcgattctctggctccaact |
| | | ctgggaacacggccaccctgaccat |
| | | cagcagggtcgaagccggggatgag |
| | | gccgactattactgtcaggtgtggg |
| | | atagtagtagtgatgtggtattcgg |
| | | cggagggaccaagctgaccgtccta |
| STLX18 VH | 119 | gaagtgcagctggttgaatctggcg |
| | | gcggattggttcagcctggcggatc |
| | | tctgagactgtcttgtgccgcctcc |
| | | ggcttcaccttctccagctacgata |
| | | tgaactgggtccgacaggccaccgg |
| | | caaaggactggaatgggtgtccgct |
| | | attggccctgccggcgacacatatt |
| | | accccggctctgtgaagggcagatt |
| | | caccatcagcagagagaacgccaag |
| | | aactccctgtacctgcagatgaaca |
| | | gcctgagagccggcgataccgccgt |
| | | gtactactgtgccagagagagatgg |
| | | cccggctacttcgatctgtggggca |
| | | gaggaacactggtcaccgtgtctag |
| | | c |
| STLX18 VL | 120 | tcctacgtgctgacccagcctcctt |
| | | ccgtgtctgttgctcctggccagac |
| | | cgccagaatcacctgtggcggaaac |
| | | aacatcggctccaagtccgtgca |
| | | ctggtatcagcagaagcctggacag |
| | | gctcctgtgctggtggtgtacgacg |
| | | actccgatagaccctctggcatccc |
| | | tgagagattctccggctccaacagc |
| | | ggcaataccgccacactgaccatct |
| | | ccagagtggaagctggcgacgaggc |
| | | cgactactactgccaagtgtgggac |
| | | tcctcctccgatcacgtggtgtttg |
| | | gcggcggaacaaagctgacagtgct |
| | | g |
| STLX21 VH | 121 | gaagttcagctggttcagtctggcg |
| | | ccgaagtgaagaaggctggcgagtc |
| | | cctgaagatctcctgcaaaggctcc |
| | | ggctactccttcacctcctactgga |
| | | tcggctgggtccgacagatgcctgg |
| | | caaaggcttggagtggatgggcatc |
| | | atctaccccggctcctctgatacca |
| | | gatactcccctagcttccagggcca |
| | | agtgaccatctccgccgacaagtct |
| | | atctccaccgcctacctgcagtggt |
| | | cctctctgaaggcttctgacaccgc |
| | | catgtactactgcgccagagacatc |
| | | accggcaacttcgactattggggcc |
| | | agggcacactggtcaccgtttctag |
| | | t |
| STLX21 VL | 122 | gacatccagatgacccagtctccat |
| | | cctctctgtccgcctctgtgggcga |
| | | cagagtgaccattacctgtcaggcc |
| | | agccaggacatctccaactacctga |
| | | actggtatcagcagaagcccggcaa |
| | | ggcccctaaggtgctgatctacgat |
| | | gcctccaacctggaaaccggcgtgc |
| | | cctctagattctccggctctggctc |
| | | tggcaccgactttacctttacaatc |
| | | tccagcctgcagcctgaggatatcg |
| | | ccacctactactgccagcagtacga |
| | | caacttccctcagctgacctttggc |
| | | ggaggcaccaaggtggaaatcaag |
| STLX22 VH | 123 | gaagttcagctggttcagtctggcg |
| | | ccgaagtgaagaaggctggcgagtc |
| | | cctgaagatctcctgcaaaggctcc |
| | | ggctactccttcacctcctactgga |
| | | tcggctgggtccgacagatgcctgg |
| | | caaaggcttggagtggatgggcatc |
| | | atctaccccggcacctctgatacca |
| | | gatactcccctagcttccagggcca |
| | | agtgaccatctccgccgacaagtct |
| | | atctccaccgcctacctgcagtggt |
| | | cctctctgaaggcttctgacaccgc |
| | | catgtactactgcgccagagacatc |
| | | accggcaacttcgactattggggcc |
| | | agggcacactggtcaccgtttctag |
| | | t |
| STLX22 VL | 124 | gacatccagatgacccagtctccat |
| | | cctctctgtccgcctctgtgggcga |
| | | cagagtgaccattacctgtcaggcc |
| | | agccaggacatctccaactacctga |
| | | actggtatcagcagaagcccggcaa |
| | | ggcccctaaggtgctgatctacgat |
| | | gcctccaacctggaaaccggcgtgc |
| | | cctctagattctccggctctggctc |
| | | tggcaccgactttacctttacaatc |
| | | tccagcctgcagcctgaggatatcg |
| | | ccacctactactgccagcagtacga |
| | | caacttccctcagctgacctttggc |
| | | ggaggcaccaaggtggaaatcaag |
| STLX31 VH | 125 | gaggttcagctggtggagtctgggg |
| | | gaggcctggtcaagcctggggggtc |
| | | cctgagactctcctgtgcagcctct |
| | | ggattcaccttcagtagctttagca |
| | | tgagctgggtccgccaggctccagg |
| | | gaaggggctggagtgggtctcatcc |
| | | ataagtagtagtagtagttacatag |
| | | actacgcagactcagtgaagggccg |
| | | attcaccatctccagagacaacgcc |
| | | aagaactcactgtatctgcaaatga |
| | | acagcctgacagccgaggacacggc |
| | | tgtgtatttctgtgcgagagatgta |
| | | ggcccctattggtacttcgatctct |
| | | ggggccgtggcaccctggtcactgt |
| | | ctcctca |
| STLX31 VL | 126 | tcctatgtgctgactcagccaccct |
| | | cggtgtcagtggccccaggacagac |
| | | ggccaggattacctgtgggggaaac |
| | | aacattggaagtaaaagtgtgcatt |
| | | ggtaccagcagaagccaggccaggc |
| | | ccctgtgttggtagtttatgatgaa |
| | | agcgaccggccttcagggatccttg |
| | | agggattttcgggttccaatttggg |
| | | gaacacggccaccctgaccatcagc |
| | | agggtcgaagccggggatgaggccg |
| | | actattactgtcaggtgtgggatag |
| | | tagtagtgatcatgtggtattcggc |
| | | ggagggaccaagctgaccgtccta |
| STLX35 VH | 127 | caggtgcagctggtggagtctgggg |
| | | gaggcgaggtccagcctgggaggtc |
| | | cctgagactctcctgtccagcctct |
| | | ggattcaccttcagtagctatggca |
| | | tgcactgggtccgccaggctccagg |
| | | caaggggctggagtgggtggcactt |
| | | atatcatatgatggaggtaataaat |
| | | actatgcagactccgtgaagggccg |
| | | attcaccatctccagagacaattcc |
| | | aagaacacgctgtatctgcaaatga |
| | | acagcctgagacctgaggacacggc |
| | | tgtatattactgtgcgagagataga |
| | | gtggggatccttgactattggggcc |
| | | agggaaccctggtcaccgtctcctc |
| | | a |
| STLX35 VL | 128 | gacatccagatgacccagtctccat |
| | | cctccctgtctgcatctgtaggaga |
| | | cagagtcaccatcacttgccaggcg |
| | | agtcaggacattagcaactatttga |
| | | attggtttcagcagaaaccagggaa |
| | | agcccctaagctcctgatctacggt |
| | | gcatccaatttggaaacaggggtcc |
| | | catcaaggttcagtggaagtggatc |
| | | tgggacagattttactttcaccatc |
| | | agcagcctgcagcctgaagatattg |
| | | caacatattactgtcaacagtatga |
| | | taccctcactttcggcggagggacc |
| | | aaggtggagatcaaa |
| STLX38 VH | 129 | caggttcagctggtgcagtctggca |
| | | ccgaagtgaagaaacctggcgcctc |
| | | cgtgaaggtgtcctgcaaggcttct |
| | | ggctacacctttaccaactactaca |
| | | tgcactgggtccgacaggcccctgg |
| | | acaaggattggaatggatgggcgtg |
| | | atcaacccttctggcggaggcacca |
| | | attacgcccagaaattccagggcag |
| | | agtgaccatgaccagagacacctcc |
| | | accagcaccgtgtacatggaactgt |
| | | ccagcctgagatccgaggacaccgc |
| | | cgtgtactactgcgccagagataag |
| | | gccccttactacggcatggatgtgt |
| | | ggggccagggcaccacagtgacagt |
| | | gtcctct |
| STLX38 VL | 130 | tcctacgtgctgacccagcctcctt |
| | | ccgtgtctgttgctcctggccagac |
| | | cgccagaatcacctgtggcggaaac |
| | | aagatcggctccaagtccgtgcact |
| | | ggtatcagcagaagcctggacaggc |
| | | tcctgtgctggtggtgtacgaggac |
| | | tccgatagaccctctggcatccctg |
| | | agagattctccggctccaacagcgg |
| | | caataccgccgctctgaccatctcc |
| | | agagttgaggctggcgacgaggccg |
| | | actactactgccaagtgtgggactc |
| | | ctcctccgatcacgtggtgtttggc |
| | | ggcggaacaaagctgacagtgctg |
| STLX41 VH | 131 | gaagtgcagctggttgaatctggcg |
| | | gcggattggttcagcctggcggatc |
| | | tctgagactgtcttgtgccgcctcc |
| | | ggcttcaccttctccagctacgata |
| | | tgaactgggtccgacaggccaccgg |
| | | caaaggactggaatgggtgtccgct |
| | | attggccctgccggcgacacatatt |
| | | accccggctctgtgaagggcagatt |
| | | caccatcagcagagagaacgccaag |
| | | aactccctgtacctgcagatgaaca |
| | | gcctgagagccggcgataccgccgt |
| | | gtactactgtgccagagagagatgg |
| | | cccggctacttcgatctgtggggca |
| | | gaggaacactggtcaccgtgtctag |
| | | c |
| STLX41 VL | 132 | tcctacgtgctgacccagcctcctt |
| | | ccgtgtctgttgctcctggccagac |
| | | cgccagaatcacctgtggcggaaac |
| | | aacatcggctccaagtccgtgcact |
| | | ggtatcagcagaagcctggacaggc |
| | | tcctgtgctggtggtgtacgacacc |
| | | tccgatagaccctctggcatccctg |
| | | agagattctccggctccaacagcgg |
| | | caataccgccacactgaccatctcc |
| | | agagtggaagctggcgacgaggccg |
| | | actactactgccaagtgtgggactc |
| | | ctcctccgatcacgtggtgtttggc |
| | | ggcggaacaaagctgacagtgctg |
| STLX42 VH | 133 | gaagtgcagctggttgaatctggcg |
| | | gcggattggttcagcctggcggatc |
| | | tctgagactgtcttgtgccgcctcc |
| | | ggcttcaccttctccagctacgata |
| | | tgaactgggtccgacaggccaccgg |
| | | caaaggactggaatgggtgtccgct |
| | | attggccctgccggcgacacatatt |
| | | accccggctctgtgaagggcagatt |
| | | caccatcagcagagagaacgccaag |
| | | aactccctgtacctgcagatgaaca |
| | | gcctgagagccggcgataccgccgt |
| | | gtactactgtgccagagagagatgg |
| | | cccggctacttcgatctgtggggca |
| | | gaggaacactggtcaccgtgtctag |
| | | c |
| STLX42 VL | 134 | tcctacgtgctgacccagcctcctt |
| | | ccgtgtctgttgctcctggccagac |
| | | cgccagaatcacctgtggcggaaac |
| | | aacatcggctccaagtccgtgcact |
| | | ggtatcagcagaagcctggacaggc |
| | | tcctgtgctggtggtgtacgacgac |
| | | aacgacagaccctctggcatccctg |
| | | agagattctccggctccaacagcgg |
| | | caataccgccacactgaccatctcc |
| | | agagtggaagctggcgacgaggccg |
| | | actactactgccaagtgtgggactc |
| | | ctcctccgatcacgtggtgtttggc |
| | | ggcggaacaaagctgacagtgctg |
| STLX43 VH | 135 | gaagtgcagctggttgaatctggcg |
| | | gcggattggttcagcctggcggatc |
| | | tctgagactgtcttgtgccgcctcc |
| | | ggcttcaccttctccagctacgata |
| | | tgaactgggtccgacaggccaccgg |
| | | caaaggactggaatgggtgtccgct |
| | | attggccctgccggcgacacatatt |
| | | accccggctctgtgaagggcagatt |
| | | caccatcagcagagagaacgccaag |
| | | aactccctgtacctgcagatgaaca |
| | | gcctgagagccggcgataccgccgt |
| | | gtactactgtgccagagagagatgg |
| | | cccggctacttcgatctgtggggca |
| | | gaggaacactggtcaccgtgtctag |
| | | c |
| STLX43 VL | 136 | tcctacgtgctgacccagcctcctt |
| | | ccgtgtctgttgctcctggccagac |
| | | cgccagaatcacctgtggcggaaac |
| | | aacatcggctccaagtccgtgcact |
| | | ggtatcagcagaagcctggacaggc |
| | | tcctgtgctggtggtgtacgacgac |
| | | tccgatagaccctctggcatccctg |
| | | agagattctccggctccaacagcgg |
| | | caataccgccacactgaccatctcc |
| | | agagtggaagctggcgacgaggccg |
| | | actactactgccaagtgtgggagtc |
| | | ctcctccgatcacgtggtgtttggc |
| | | ggcggaacaaagctgacagtgctg |
| STLX44 VH | 137 | gaagtgcagctggttgaatctggcg |
| | | gcggattggttcagcctggcggatc |
| | | tctgagactgtcttgtgccgcctcc |
| | | ggcttcaccttctccagctacgata |
| | | tgaactgggtccgacaggccaccgg |
| | | caaaggactggaatgggtgtccgct |
| | | attggccctgccggcgacacatatt |
| | | accccggctctgtgaagggcagatt |
| | | caccatcagcagagagaacgccaag |
| | | aactccctgtacctgcagatgaaca |
| | | gcctgagagccggcgataccgccgt |
| | | gtactactgtgccagagagagatgg |
| | | cccggctacttcgatctgtggggca |
| | | gaggaacactggtcaccgtgtctag |
| | | c |
| STLX44 VL | 138 | tcctacgtgctgacccagcctcctt |
| | | ccgtgtctgttgctcctggccagac |
| | | cgccagaatcacctgtggcggaaac |
| | | aacatcggctccaagtccgtgcact |
| | | ggtatcagcagaagcctggacaggc |
| | | tcctgtgctggtggtgtacgacgac |
| | | tccgatagaccctctggcatccctg |
| | | agagattctccggctccaacagcgg |
| | | caataccgccacactgaccatctcc |
| | | agagtggaagctggcgacgaggccg |
| | | actactactgccaagtgtggacctc |
| | | ctcctccgatcacgtggtgtttggc |
| | | ggcggaacaaagctgacagtgctg |
| STLX45 VH | 139 | gaagtgcagctggttgaatctggcg |
| | | gcggattggttcagcctggcggatc |
| | | tctgagactgtcttgtgccgcctcc |
| | | ggcttcaccttctccagctacgata |
| | | tgaactgggtccgacaggccaccgg |
| | | caaaggactggaatgggtgtccgct |
| | | attggccctgccggcgacacatatt |
| | | accccggctctgtgaagggcagatt |
| | | caccatcagcagagagaacgccaag |
| | | aactccctgtacctgcagatgaaca |
| | | gcctgagagccggcgataccgccgt |
| | | gtactactgtgccagagagagatgg |
| | | cccggctacttcgatctgtggggca |
| | | gaggaacactggtcaccgtgtctag |
| | | c |
| STLX45 VL | 140 | tcctacgtgctgacccagcctcctt |
| | | ccgtgtctgttgctcctggccagac |
| | | cgccagaatcacctgtggcggaaac |
| | | aacatcggctccaagtccgtgcact |
| | | ggtatcagcagaagcctggacaggc |
| | | tcctgtgctggtggtgtacgacacc |
| | | tctgatagaccctctggcatccctg |
| | | agagattctccggctccaacagcgg |
| | | caataccgccacactgaccatctcc |
| | | agagtggaagctggcgacgaggccg |
| | | actactactgccaagtgtgggagtc |
| | | ctcctccgatcacgtggtgtttggc |
| | | ggcggaacaaagctgacagtgctg |
| STLX46 VH | 141 | gaagtgcagctggttgaatctggcg |
| | | gcggattggttcagcctggcggatc |
| | | tctgagactgtcttgtgccgcctcc |
| | | ggcttcaccttctccagctacgata |
| | | tgaactgggtccgacaggccaccgg |
| | | caaaggactggaatgggtgtccgct |
| | | attggccctgccggcgacacatatt |
| | | accccggctctgtgaagggcagatt |
| | | caccatcagcagagagaacgccaag |
| | | aactccctgtacctgcagatgaaca |
| | | gcctgagagccggcgataccgccgt |
| | | gtactactgtgccagagagagatgg |
| | | cccggctacttcgatctgtggggca |
| | | gaggaacactggtcaccgtgtctag |
| | | c |
| STLX46 VL | 142 | tcctacgtgctgacccagcctcctt |
| | | ccgtgtctgttgctcctggccagac |
| | | cgccagaatcacctgtggcggaaac |
| | | aacatcggctccaagtccgtgcact |
| | | ggtatcagcagaagcctggacaggc |
| | | tcctgtgctggtggtgtacgacacc |
| | | tctgatagaccctctggcatccctg |
| | | agagattctccggctccaacagcgg |
| | | caataccgccacactgaccatctcc |
| | | agagtggaagctggcgacgaggccg |
| | | actactactgccaagtgtggacctc |
| | | ctcctccgatcacgtggtgtttggc |
| | | ggcggaacaaagctgacagtgctg |
| STLX47 VH | 36 | gaagtgcagctggttgaatctggcg |
| | | gcggattggttcagcctggcggatc |
| | | tctgagactgtcttgtgccgcctcc |
| | | ggcttcaccttctccagctacgata |
| | | tgaactgggtccgacaggccaccgg |
| | | caaaggactggaatgggtgtccgct |
| | | attggccctgccggcgacacatatt |
| | | accccggctctgtgaagggcagatt |
| | | caccatcagcagagagaacgccaag |
| | | aactccctgtacctgcagatgaaca |
| | | gcctgagagccggcgataccgccgt |
| | | gtactactgtgccagagagagatgg |
| | | cccggctacttcgatctgtggggca |
| | | gaggaacactggtcaccgtgtctag |
| | | c |
| STLX47 VL | 37 | tcctacgtgctgacccagcctcctt |
| | | ccgtgtctgttgctcctggccagac |
| | | cgccagaatcacctgtggcggaaac |
| | | aacatcggctccaagtccgtgcact |
| | | ggtatcagcagaagcctggacaggc |
| | | tcctgtgctggtggtgtacgacgac |
| | | aacgacagaccctctggcatccctg |
| | | agagattctccggctccaacagcgg |
| | | caataccgccacactgaccatctcc |
| | | agagtggaagctggcgacgaggccg |
| | | actactactgccaagtgtgggagtc |
| | | ctcctccgatcacgtggtgtttggc |
| | | ggcggaacaaagctgacagtgctg |
| STLX48 VH | 38 | gaagtgcagctggttgaatctggcg |
| | | gcggattggttcagcctggcggatc |
| | | tctgagactgtcttgtgccgcctcc |
| | | ggcttcaccttctccagctacgata |
| | | tgaactgggtccgacaggccaccgg |
| | | caaaggactggaatgggtgtccgct |
| | | attggccctgccggcgacacatatt |
| | | accccggctctgtgaagggcagatt |
| | | caccatcagcagagagaacgccaag |
| | | aactccctgtacctgcagatgaaca |
| | | gcctgagagccggcgataccgccgt |
| | | gtactactgtgccagagagagatgg |
| | | cccggctacttcgatctgtggggca |
| | | gaggaacactggtcaccgtgtctag |
| | | c |
| STLX48 VL | 39 | tcctacgtgctgacccagcctcctt |
| | | ccgtgtctgttgctcctggccagac |
| | | cgccagaatcacctgtggcggaaac |
| | | aacatcggctccaagtccgtgcact |
| | | ggtatcagcagaagcctggacaggc |
| | | tcctgtgctggtggtgtacgacgac |
| | | aacgacagaccctctggcatccctg |
| | | agagattctccggctccaacagcgg |
| | | caataccgccacactgaccatctcc |
| | | agagtggaagctggcgacgaggccg |
| | | actactactgccaagtgtggacctc |
| | | ctcctccgatcacgtggtgtttggc |
| | | ggcggaacaaagctgacagtgctg |

Table 1G provide the nucleotide sequences encoding the VH and VL regions of the anti-IL-8 clones, wherein the CDR regions are marked as follows: (a) the Kabat modeled CDRs are encoded by the sequences in Bold and (b) the IMGT modeled CDRs are encoded by the sequences in Italics (5′-3′ CDR1, CDR2, CDR3 for each of VH and VL listed above).

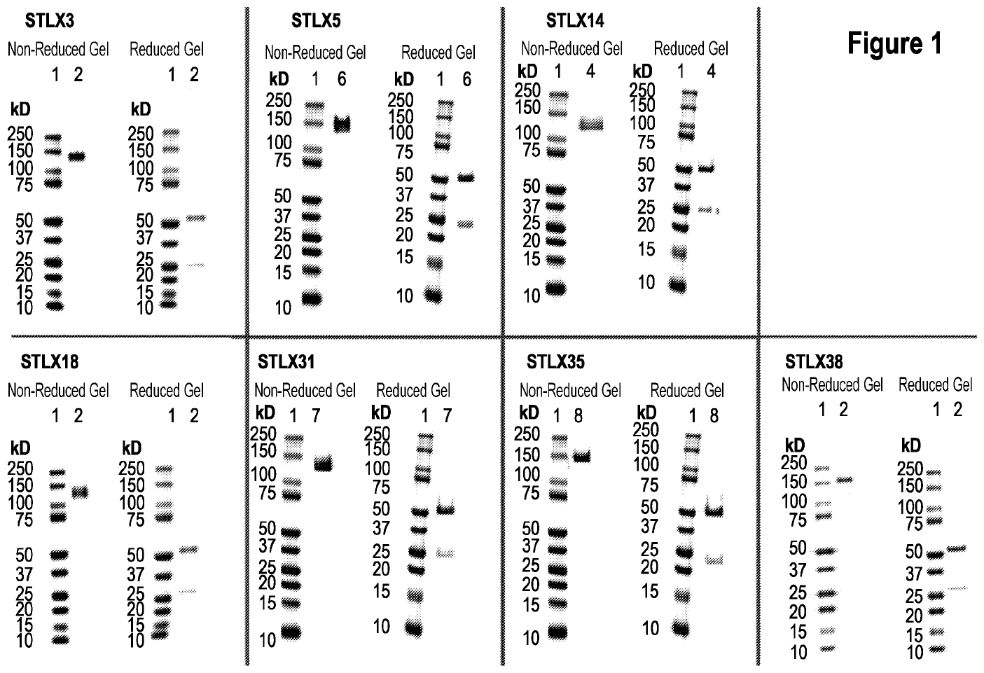

Expression of antibody molecules was studied under non-reducing and reducing conditions. An intact antibody migrating at approximately 150 kDa, as typical for IgG, was detected in all antibody samples that run under non-reducing conditions (FIG. 1—left-hand lanes). A band migrating at 50 kDa, as typical of IgG heavy chain and a lower band at 25 kDa, as typical for IgG light chain, were detected in all antibody samples that ran under reducing conditions (FIG. 1—right-hand lanes).

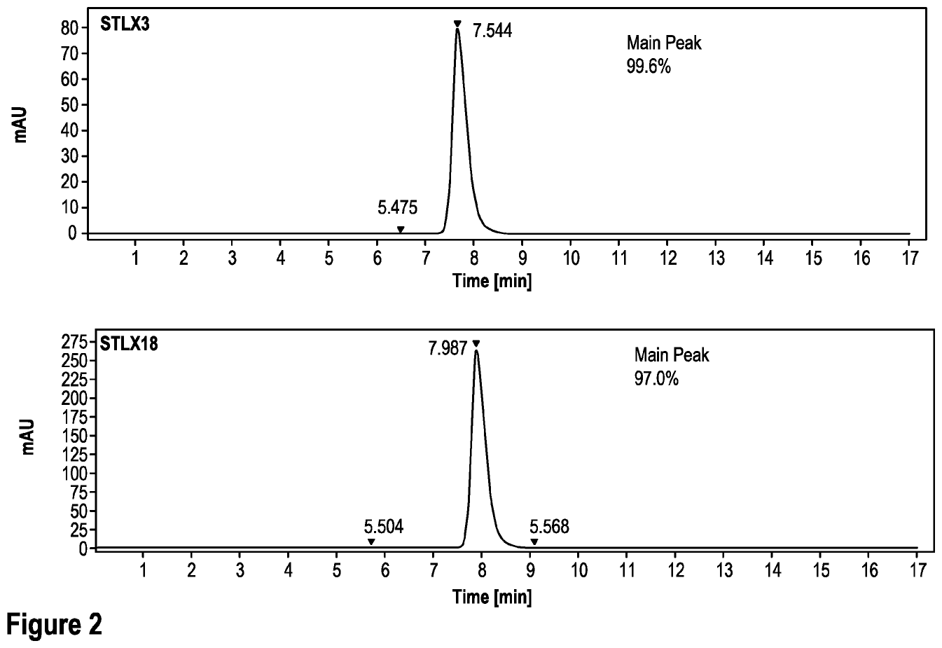

Size-exclusion analysis was conducted in HPLC. Profiles of antibodies showed a symmetrical main peak with retention time between 7.6-7.9 minutes, as typical for intact IgG monomer. (FIG. 2) All samples showed >95% main peak, as determined by AUC, indicating very little soluble aggregate or clipping degradation by size exclusion chromatography.

The recombinant Ab clones generated had different light chains wherein clones STLX3, STLX5 and STLX35 harbor× (kappa) light chains, and STLX14, STLX18, STLX31 and STLX38 harbor λ (lambda) light chains.

### Example 3: Binding mAbs to Human and Cynomolgus IL-8

Objective: Analyze the binding kinetics of the mAbs generated, for binding to human and cynomolgus IL-8. Analysis of cross-reactivity to monkey IL-8 is important as this allows toxicity studies to be carried out in non-human primates using the clinical candidate directly, rather than in chimpanzee or using a surrogate molecule. Such toxicity studies may be utilized to provide relevant safety assessments.

Methods: Binding analysis was performed using ELISA or Octect, as described above.

Results:

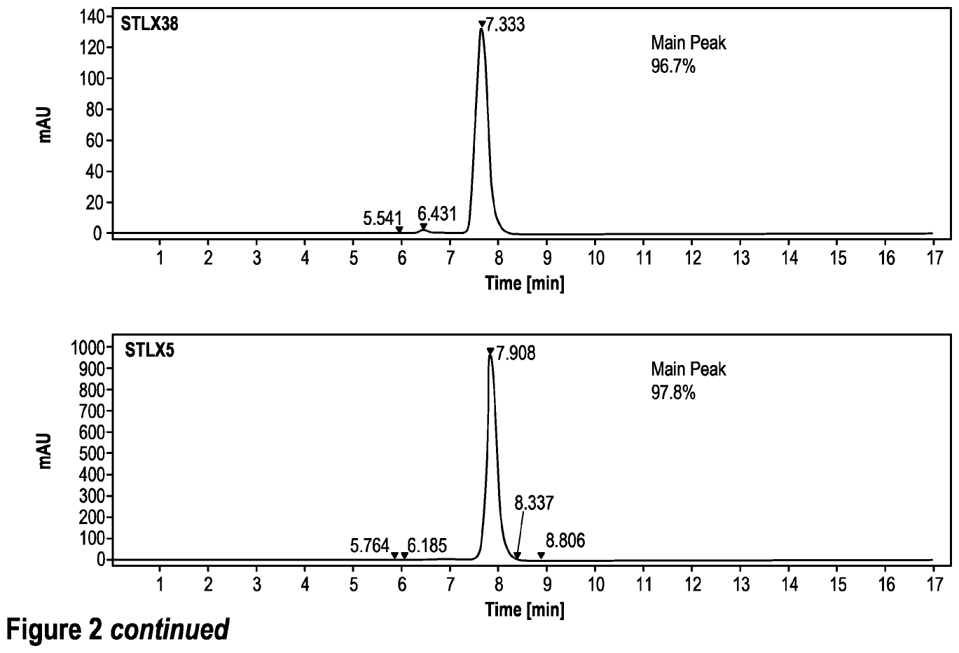

The results presented here are for mAb clones STLX3, STLX5, STLX14, STLX18, STLX31, and STLX35. Table 2 (below) and FIG. 3 demonstrate binding of the mAb to human and cynomolgus IL-8 based on ELISA results. Table 2 presents the binding kinetics of the mAbs to human and cynomolgus IL-8 as determined by cell-free ELISA. EC₅₀ values were calculated and are presented at the range of pM.

TABLE 2
Binding of mAbs to human and cynomolgus IL-8 by ELISA

| | Human IL-8 | Cynomolgus IL-8 |
|---|---|---|
| Sample ID | EC₅₀ (pM) | EC₅₀ (pM) |
| STLX3 | 201 | 169 |
| STLX5 | 184 | 165 |
| STLX14 | 170 | 166 |
| STLX18 | 411 | 303 |
| STLX31 | 218 | 243 |
| STLX35 | 298 | 229 |

The data presented in FIG. 3 and Table 3 demonstrates mAbs binding to human and cynomolgus IL-8 in dose-dependent manner.

Table 3 (below) presents the binding kinetics of the mAbs to human and cynomolgus IL-8 as determined by Octect, wherein the affinity of each antibody is represented by its Kd values. The data presented in Table 3 includes the binding on and off rates.

TABLE 3
Binding of mAbs to human and cynomolgus IL-8 by Octect

| | Human IL-8 Affinity | | | Cynomolgus IL-8 Affinity | | |
|---|---|---|---|---|---|---|
| Sample ID | Kd(M) | kon(1/2) | kdis(1/s) | Kd(M) | kon(1/2) | kdis(1/s) |
| STLX3 | 2.01E−11 | 1.13E+06 | 2.26E−05 | 6.43E−10 | 2.39E+04 | 1.54E−05 |
| STLX5 | 1.09E−10 | 1.35E+06 | 1.47E−04 | 5.94E−10 | 1.55E+05 | 9.20E−05 |
| STLX14 | 5.66E−11 | 1.22E+06 | 6.88E−05 | 6.62E−10 | 1.12E+05 | 7.43E−05 |
| STLX18 | 8.71E−11 | 1.04E+06 | 9.01E−05 | 9.83E−09 | 1.43E+04 | 1.41E−04 |
| STLX31 | 6.15E−10 | 1.40E+06 | 8.60E−04 | 5.69E−09 | 1.31E+05 | 7.42E−04 |
| STLX35 | <1.0E−12 | 1.11E+06 | <1.0E−07 | <1.0E−12 | 5.97E+04 | <1.0E−07 |

Summary: The mAb clones analyzed show a range of IL-8 binding affinities between 1-600 E-12M as demonstrated by Octet method. The on-rate for the mAb clones tested were greater than 1.0E+6/s and the off-rate was up to 8.6E-41/s. Comparison of binding to the human and cynomolgus IL-8 showed EC50 values of no greater than 2-fold difference.

### Example 4: Inhibition of IL-8 Binding to Cells Surface Receptors and IL-8-Induced NFκB Activity by mAb Clones

Objective: To analyze the functional activity of the mAb clones.

Methods: The interaction of IL-8 with CXCR2 receptors in the presence of mAb clones was measured as described in Example 1. IL-8-induced NKκB reporter assays were used to determined IL-8-induced NFκB activity in the presence of different mAb clones as described in Example 1. IL-8 induced CXCR2 internalization was measured by flow cytometry as described in Example 1.

Results:

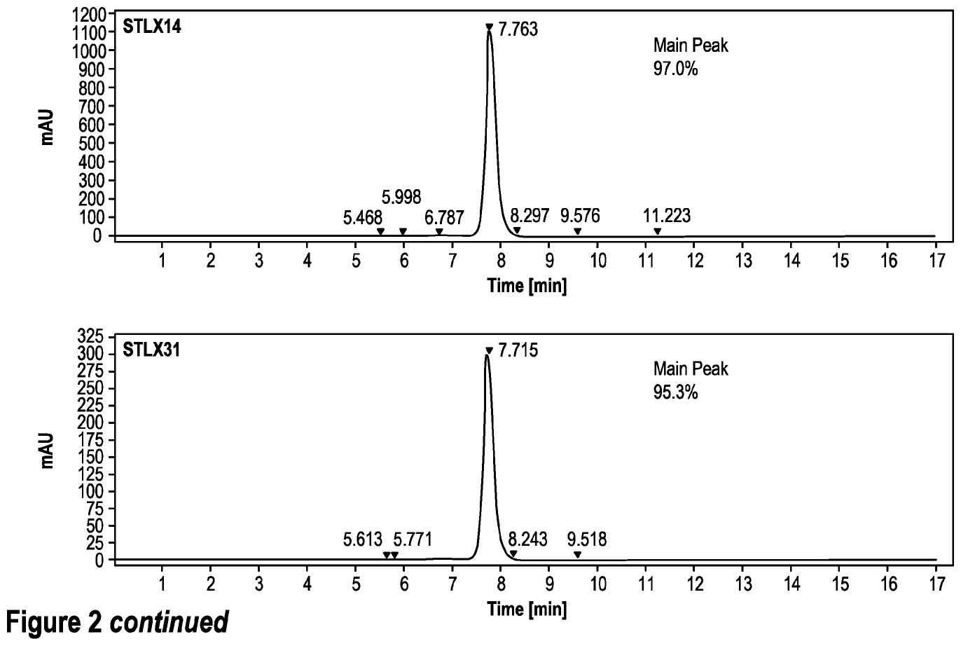

Table 4 and FIG. 4 present data demonstrating the ability of the mAb clones to block IL-8 binding to its cell surface receptor, CXCR2.

TABLE 4
Blocking IL-8 binding to its cell surface receptors, CXCR2
by mAbs - FACS blocking of IL-8-Fc binding to CXCR2

| | IC50 |
|---|---|
| Sample ID | (nM) |
| STLX3 | 6.2 |
| STLX5 | 11.1 |
| STLX14 | 10.3 |
| STLX18 | 4.0 |
| STLX31 | 12.2 |
| STLX35 | 26.1 |

The effect of mAbs on blocking the binding of IL-8-Fc to HEK293 cells, stably expressing CXCR2, was measured in cell-based assay by flow cytometry. IC50 values were calculated and are presented in Table 4 at the range of nM. FIG. 4 shows that the mAb clones promoted blocking of IL-8 binding to CXCR2 in a dose dependent manner.

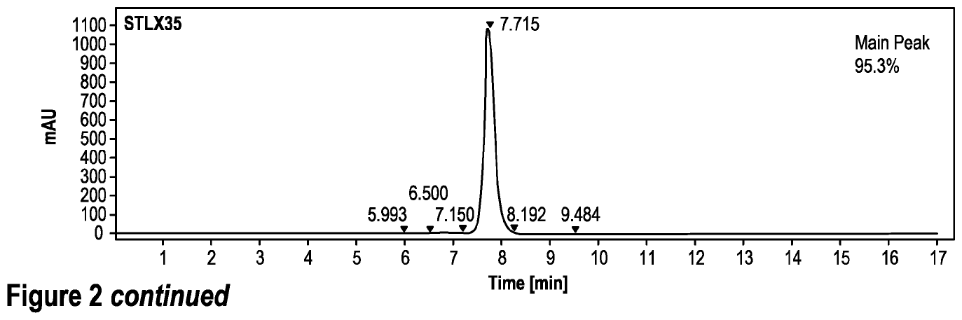

Not only did the mAb clones block IL-8 binding to CXCR2 receptors, but Table 5 and FIG. 5 present data showing that these antibodies also had the ability to inhibit IL-8-induced NFκB activity in tissue culture cells.

The effect of mAb clones on blocking IL-8-induced NFκB activity was monitored in HEK293-CXCR1 cell-based reporter assay. IC50 values of each antibody were calculated and the data is presented in Table 5, wherein IC50 values were in the range of nM.

TABLE 5
Inhibition of IL-8-induced NFκB activity by mAbs -

| | IC50 |
|---|---|
| Sample ID | (nM) |
| STLX3 | 5.4 |
| STLX5 | 5.2 |
| STLX14 | 12.9 |
| STLX18 | 6.6 |
| STLX31 | 66.0 |
| STLX35 | 5.7 |

FIG. 5 presents the data from an analysis of blocking of IL-8-induced NFκB activity by mAbs as measured in a HEK293-CXCR1 cell-based reporter assay. The data shows that the mAbs promoted blocking of NFκB activity in a dose-dependent manner.

Summary: mAb clones were able to affect IL-8 binding and activity of both CXCR1 and CXCR2 receptors, wherein the blocking potency on CXCR2 receptors as measured by a flow-based assay yielded IC50 values in the range between 3-26 nM, while for CXCR1 the NFκB reporter assay yielded IC50 values in the range between 5-66 nM.

### Example 5: Expression of CXCR1 and CXCR2 Receptors on Cancer Cells, and Inhibition of IL-8-Induced CXCR2 Internalization and Inhibition of Growth of Cancer Cells by mAb Clones

Objective: Analyze the effect of the mAb clones on cancer cells (primary acute myeloid leukemia (AML) cancer cells and haematological and solid tumor cell lines).

Methods: The detection of CXCR1 and CXCR2 on the cell surface of cancer cells was performed as described in Example 1 using flow cytometry and immunohistochemistry. Detection of IL-8 secretion by cancer cell lines was performed using ELISA, as described in Example 1. Further viability assays used are those described in Example 1.

Results:

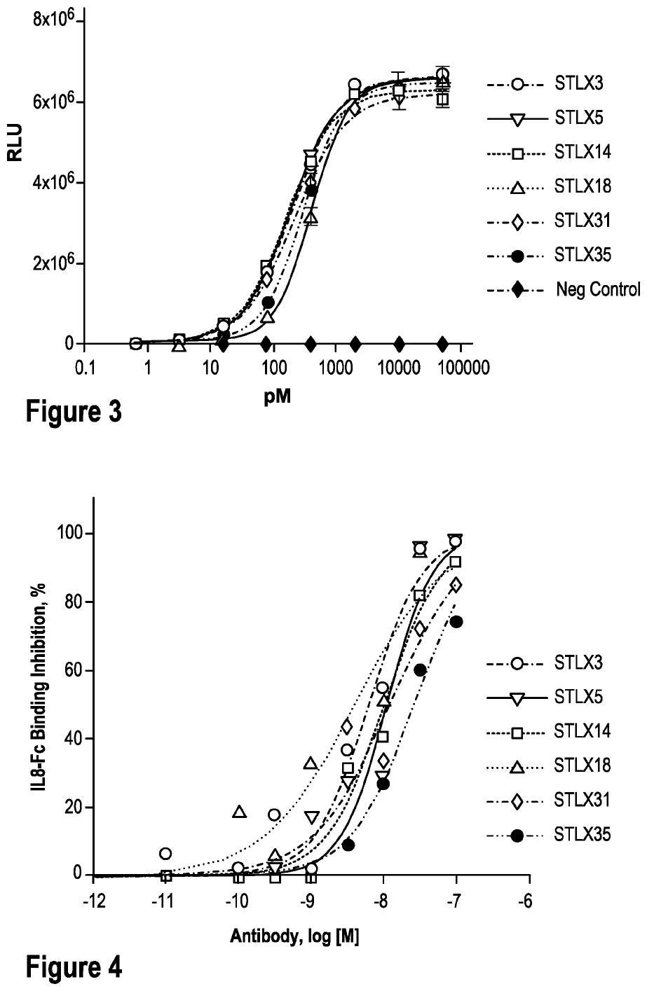

Cell surface expression analysis showed detection of CXCR2 receptors but not CXCR1 receptors on the majority of AML cell lines analyzed (THP-1, OCI-AML5, MOLM-13, MOLM-14, HL-60, HEL, OCI-AML2, OCI-AML3, MV411, and KG-1a) (Table 6), while osteosarcoma cells lines (143B, SaOS-2, U-2 OS, MG-63, OS17) and a pancreatic cancer cell line (PANC-1) expressed both CXCR2 and CXCR1 receptors (Table 7; FIG. 6).

TABLE 6
Cell Surface Detection of CXCR1 and CXCR2 by FACS

| Cell line ID | Cell Type | CXCR1 | CXCR2 |
|---|---|---|---|
| THP-1 | AML | − | ++ |
| OCI-AML5 | AML | − | +++ |
| MOLM-13 | AML | − | ++ |
| MOLM-14 | AML | − | + |
| HL-60 | AML | − | + |
| HEL | AML | − | − |
| OCI-AML2 | AML | − | + |
| OCI-AML3 | AML | − | + |
| MV411 | AML | − | + |
| KG-1a | AML | − | − |

Table 6 shows that expression of CXCR1 receptor was not detected in cell lines, analyzed by flow cytometry as indicated with the (−) symbol. Expression of CXCR2 receptor was detected by flow cytometry in most cell lines at varying levels as indicated with the (+) symbol. Highest expression level is illustrated with (+++) and lowest is illustrated with (+).

TABLE 7
Cell surface expression of CXCR1 and CXCR2
in solid tumor cell lines by IHC

| Cell line ID | Cell Type | CXCR1 | CXCR2 |
|---|---|---|---|
| 143B | Osteosarcoma | ++ | +++ |
| SaOS-2 | Osteosarcoma | + | +++ |
| U-2 OS | Osteosarcoma | ++ | + |
| MG-63 | Osteosarcoma | + | + |
| OS17 | Osteosarcoma | ++ | +++ |
| PANC-1 | Pancreatic cancer | +++ | +++ |

Staining of solid tumor cell lines (osteosarcoma and pancreatic) showed that expression of CXCR1 and CXCR2 receptors was detected at varying levels in osteosarcoma and pancreatic tumor cell lines by IHC (FIG. 6). A no primary antibody condition was used as a negative control. Table 7 tabulates the expression of CXCR1 and CXCR2 receptors detected on the cell surface by IHC in all osteosarcoma and pancreatic cell lines tested at varying levels as indicated with the (+) symbol. Highest expression level is illustrated with (+++) and lowest is illustrated with (+).

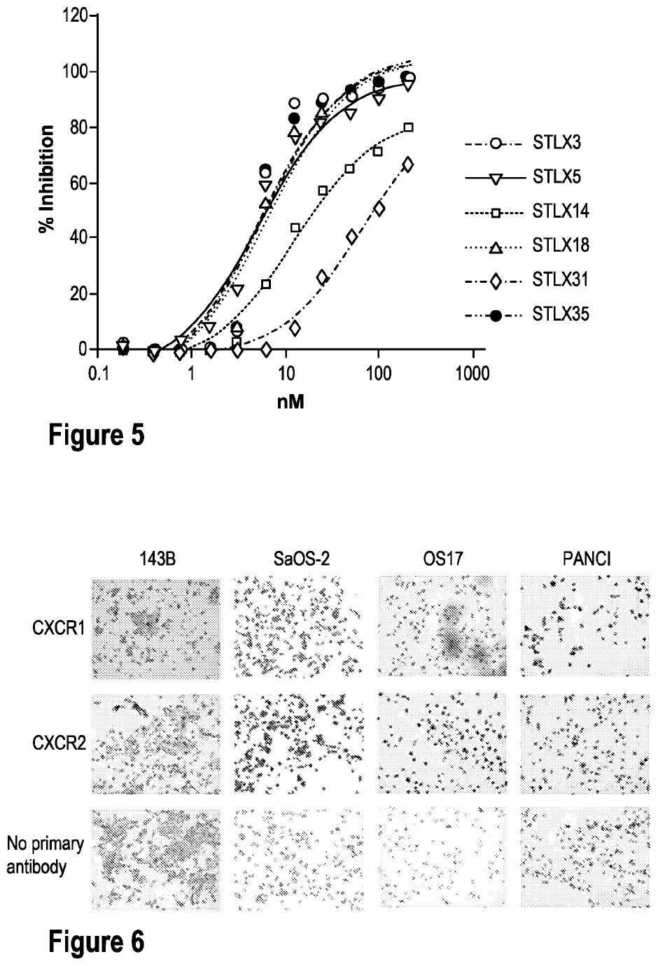

With the recognition that IL-8 may function in paracrine and autocrine modes, a follow-up analysis examined the expression of IL-8 in different solid tumor and haematological tumor cell lines. Table 8 and FIG. 7 provide data demonstrating IL-8 secretion in both osteosarcoma and AML cell lines. FIG. 7 shows the results of monitoring secretion of IL-8 by ELISA in the supernatants of MG-63, U2-OS, SAOS-2 and 143-B osteosarcoma cells. Secretion of IL-8 was monitored in MG-63, U2-OS and 143-B cells in a time-dependent manner. The lowest levels of IL-8 secretion were detected in SAOS-2 cells and were not increased during a time-course of 96 hours. The presence of a higher percentage of FBS in growth media correlated with higher IL-8 secretion levels. Table 8 presents the IL-8 data for both osteosarcoma cell lines and AML cell lines.

TABLE 8
Secretion levels of IL-8 in cell lines by ELISA

| | | IL-8 secretion level |
|---|---|---|
| Cell line ID | Cell Type | (ELISA) |
| 143B | Osteosarcoma | +++ |
| SaOS-2 | Osteosarcoma | + |
| U-2 OS | Osteosarcoma | ++ |
| MG-63 | Osteosarcoma | ++ |
| OS17 | Osteosarcoma | +++ |
| MOLM13 | AML | ++ |
| MOLM14 | AML | + |
| MV-411 | AML | ++ |
| OCIAML | AML | + |
| OCIAML3 | AML | +++ |
| THP-1 | AML | ++ |

Table 8 shows that secretion of IL-8 was detected by ELISA in all cell lines tested at varying levels as indicated with the (+) symbol. Highest secretion level is illustrated with (+++) and lowest is illustrated with (+).

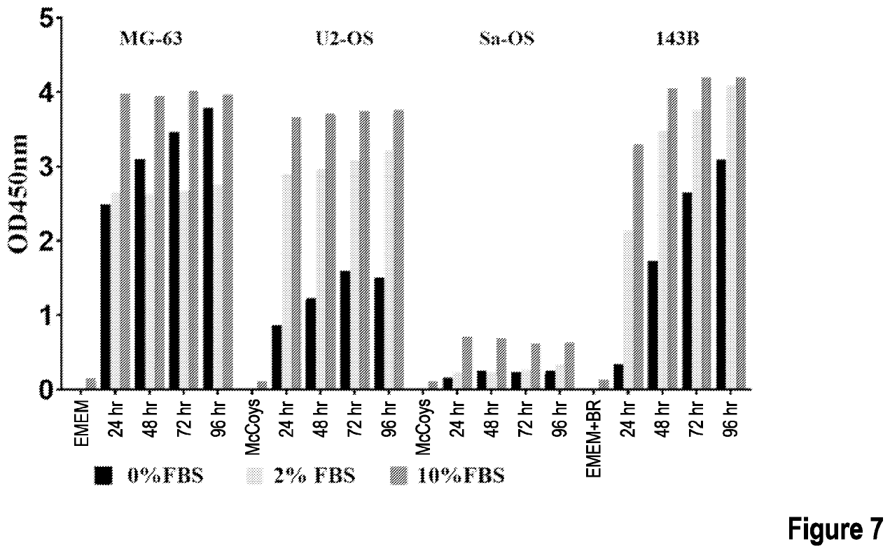

Based on the data that CXCR2 receptors were present on acute monocytic leukemia (AML) cells, inhibition of IL-8-induced CXCR2 internalization by the mAb clones was examined in AML cells (FIG. 8). FIG. 8 shows that in THP-1 cells, CXCR2 receptor internalization was induced by IL-8 treatment, while CXCR2 internalization was blocked in a dose-dependent manner by cotreatment with mAb clones STLX3, STLX5, STLX14, STLX18, STLX31, STLX35, and STLX38. The percentage of control was calculated by [1−(X−min)/(max−min)]*100 and displayed in the graph, where IL-8 plus antibody was represented by light grey, unstimulated was represented as max and IL-8 stimulation alone was represented as min.

Next, the ability of the mAbs to inhibit cancer cell growth was measured. Table 9 presents data showing inhibition of patient-derived AML cells by the mAbs. As used throughout, in some embodiments an mAb clone is identified with the letters STX followed by a number and in other embodiments, as STLX followed by the numbers. A skilled provisional would appreciate the for each mAb clone STX3 and STLX3 identify the same clone. This pattern hold true for all of the clones listed here.

TABLE 9
Cell Growth Inhibition of Patient-derived AML Samples
% Inhibition of cell growth at highest dose

| | Primary Cell Line | | | | | | |
|---|---|---|---|---|---|---|---|
| mAb | 2227 | 2229 | 2234 | 2239 | 2774 | 2775 | 3440 |
| STX3 | | | | | | | |
| STX5 | | | | | | | |
| STX14 | | | | | | | |
| STX18 | | | | | | | |
| STX31 | | | | | | | |
| STX35 | | n/a | | | | | |
| STX38 | | n/a | | | | | |

```
  >30%
  >3%, ≤30%
  ≤3%
```

The effect of mAbs on inhibiting the growth of patient-derived AML cells was monitored by cell proliferation assay and measured by Cell-Titer Glo. Patient-derived AML cells were treated with each antibody at the concentration of 20 μg/ml for 6 days and the percentage of growth inhibition was calculated and presented with different colors. Inhibition higher than 30% is illustrated with black circles, inhibition between 3% and 30% with grey and inhibition lower than 3% with white.

The ability of the mAbs to inhibit cancer cell growth in haematological and solid tumor cell lines will also be measured. The effect of mAbs on inhibiting the growth of haematological and solid tumor cell lines will be monitored by cell proliferation assay and measured by CellTiter-Glo. Cell lines of bladder, leukemia, lymphoma, myeloma, bone, HN/pharynx or lung origin (squamous cell carcinoma of the head and neck (SCCHN) and non-small-cell lung carcinoma (NSCLC)) will be treated with each antibody at the concentration of 200 μg/ml for 3 days and the percentage of growth inhibition will be calculated and presented in a table.

Summary: The results demonstrate the presence CXCR1 and CXCR2 receptors on cancer cells and the ability of the mAb clones generated to inhibit IL-8-induction of CXCR2 internalization in an AML cancer cell line. Moreover, the mAbs inhibited growth in patient derived AML cancer cells.

The results presented in Examples 2-5 show that fully human monoclonal antibodies directed again IL-8 were generated and provided functional activities. These mAbs blocked the ability of IL-8 to bind to its cell-surface receptors, and thus interfere with IL-8's ability to transduce a cellular signal. Use of these mAbs also inhibited growth of AML primary cancer cells, showing their relevancy for cancer therapies.

### Example 6: STLX18 Inhibits IL-8-Induced AKT and ERK Phosphorylation in Neutrophils

Objective: To examine the effect of IL-8 antibodies on IL-8 induced signaling in human neutrophils.

Methods: Human neutrophils isolated from whole blood were activated with IL-8 in the presence of an IL-8 neutralizing antibody (STLX18) or a control IgG1 antibody. Cell lysates were probed by Western Blot and IL-8 induced downstream signaling was monitored with antibodies against phospho-specific ERK and AKT. The signal was detected by chemiluminescence with exposure to autoradiographic film. An antibody to actin was used as a loading control.

Results:

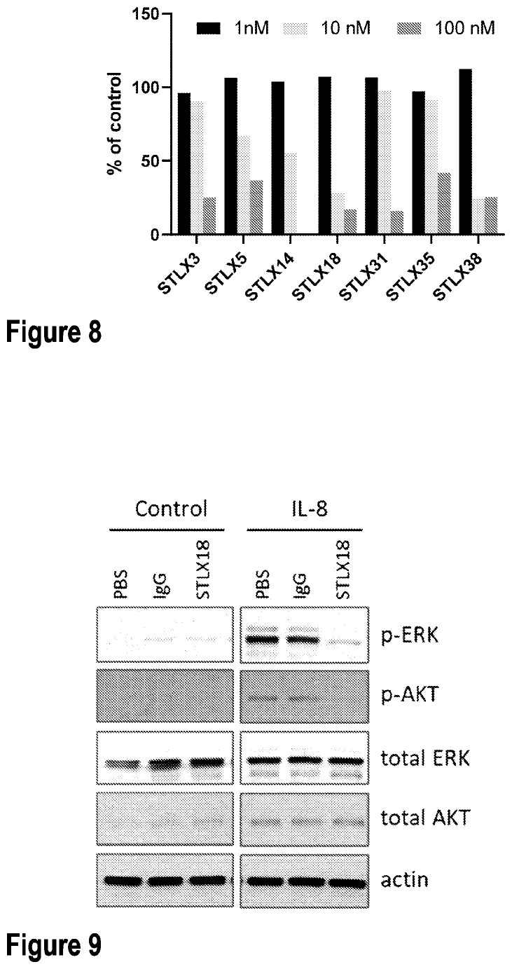

FIG. 9 shows that STLX18 monoclonal antibody inhibited IL-8 induced downstream signaling in human neutrophils, as monitored by Western Blot using phospho-specific antibodies against ERK and AKT. There is no background signaling in the absence of IL-8. The IgG1 control has the same backbone as STLX18 antibody but does not target IL-8.

Summary: The results demonstrate that the IL-8 neutralizing antibody STLX18 inhibited IL-8 induced phospho-signaling in primary human neutrophils. This suggests that STLX18 inhibited IL-8 signaling through its cell surface receptors.

### Example 7: STLX18 Inhibits Human Neutrophil Migration Towards IL-8 In-Vitro

Objective: To examine the in vitro activity of IL-8 antibodies on the chemotaxis of neutrophils.

Methods: Human neutrophils isolated from whole blood were placed in the top chamber of multi-well inserts with IL-8 in the bottom chamber. The bottom chamber also contained either an IL-8 neutralizing antibody (STLX18) or a control IgG1 antibody. Neutrophil migration was assessed by either a live cell imaging software or cell counts.

Results:

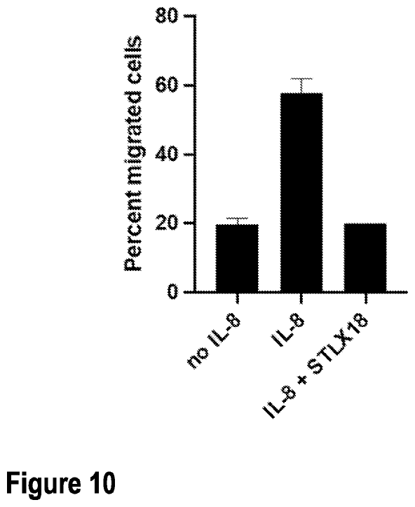

Human neutrophil migration, towards an IL8 source, across a porous membrane was inhibited by the STLX18 antibody. Addition of IL8 to the bottom chamber of a trans-well plate increased human neutrophil migration across the membrane and was inhibited by the co-incubation of STLX18 antibody and IL-8 in the bottom chamber (FIG. 10). Neutrophil migration was assessed by Incucyte® Live-Cell Analysis System.

Summary: These results demonstrate that STLX18 antibody inhibited neutrophil migration towards IL-8 in an in-vitro chemotaxis assay. This suggests that STLX18 may provide a therapeutic effect in neutrophil-driven inflammatory conditions, including cancer.

### Example 8: STLX18 Inhibits Knee Swelling in a Rabbit Model of Gouty Knee Inflammation

Objective: To analyze the in vivo activity of IL-8 antibodies in a gouty knee inflammation model.

Methods: Monosodium urate (MSU) crystals injected into a knee joint result in swelling and inflammation. Rabbit knees were pre-treated with either saline or an IL-8 neutralizing antibody (STLX18). The right knee of each rabbit was injected with MSU crystals, and the left knee was injected with saline. Knee diameter was measured at various time points using calipers.

Results:

Binding analysis of STLX18 antibody to rabbit IL-8 showed that the STLX18 antibody recognized rabbit IL-8. (Data not shown)

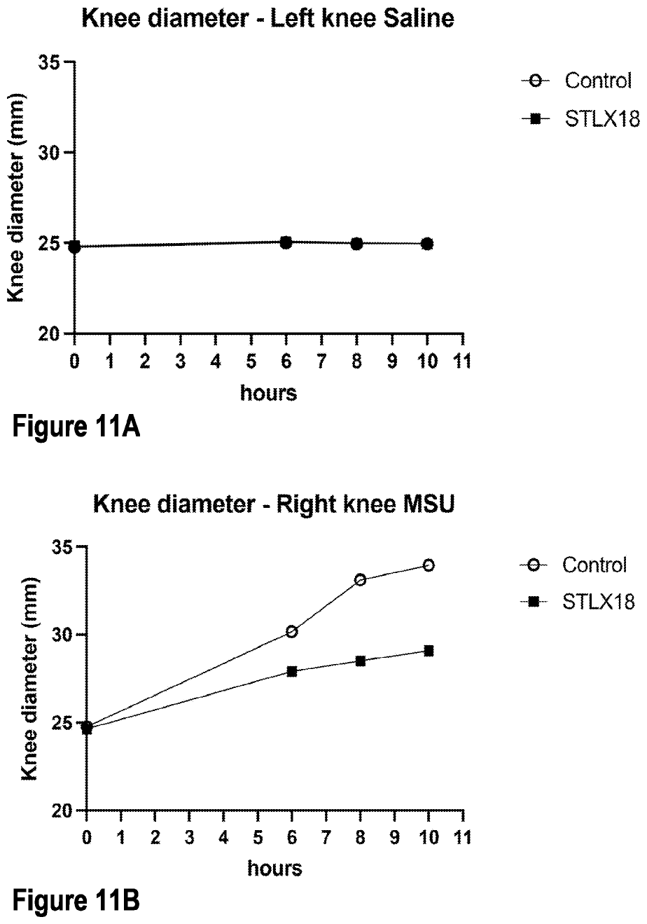

Rabbit knee joints injected with MSU crystals increased in diameter shortly after injection. Treatment with STLX18 antibody reduced the swelling of MSU injected joints (FIG. 11B) while control joints infused with saline did not change in size (FIG. 11A). Knee diameter was determined by caliper measurements at indicated times after injection.

Summary:

These results indicate that IL-8 antibodies were able to reduce knee swelling induced by MSU crystals and may provide a therapeutic treatment for diseases and conditions associated with inflammation.

While certain features of the IL-8 antibodies and uses thereof have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1.-30. (canceled)

31. An isolated anti-IL-8 antibody having complementarity determining region (CDR) sequences as set forth in Table 1F, wherein each antibody comprises a heavy chain variable region (VH) having heavy chain complementarity determining region (HCDR) 1, HCDR2 and HCDR3, and a light chain variable region (VL) having light chain complementarity determining region (LCDR) 1, LCDR2 and LCDR3, wherein said HCDR1, HCDR2, HCDR3, LCDR1, LCDR2 and LCDR3 for each of said antibody comprise the amino acid sequences as set forth in:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109.

32. The isolated anti-IL-8 antibody of claim 31, wherein each antibody comprises a heavy chain variable region (VH)—light chain variable region (VL) pair, and wherein the amino acid sequences of the VH-VL pair are selected from the paired sequences set forth in SEQ ID NOs: 8 and 9, SEQ ID NOs: 2 and 3, SEQ ID NOs: 4 and 5, SEQ ID NOs: 6 and 7, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35.

33. The isolated anti-IL-8 antibody of claim 31, wherein said antibody comprises an IgG, an Fv, an scFv, an Fab, an F(ab′)₂, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody.

34. A composition comprising the isolated anti-IL-8 antibody of claim 31, and a pharmaceutically acceptable carrier.

35. An isolated polynucleotide sequence encoding a heavy chain variable region (VH) of an anti-IL-8 antibody comprising the complementarity determining regions (HCDR) of said VH as set forth in Table 1F and encoding a light chain variable region (VL) of the anti-IL-8 antibody comprising the complementarity determining regions (LCDR) of said VL as set forth in Table 1F, wherein said heavy chain variable region comprises heavy chain complementarity determining region (HCDR) 1, HCDR2 and HCDR3, and said light chain variable region comprises light chain complementarity determining region (LCDR) 1, LCDR2 and LCDR3, wherein the amino acid sequences of said HCDR1, HCDR2, HCDR3, LCDR1, LCDR2 and LCDR3 for each of said VH and VL are set forth in:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109.

36. The isolated polynucleotide sequence of claim 35, wherein each polynucleotide encodes a heavy chain variable region (VH)—light chain variable region (VL) pair, and wherein the amino acid sequences encoded by the VH-VL pair are selected from the paired amino acid sequences set forth in SEQ ID NOs: 8 and 9, SEQ ID NOs: 2 and 3, SEQ ID NOs: 4 and 5, SEQ ID NOs: 6 and 7, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35.

37. The isolated polynucleotide sequence of claim 36, wherein the polynucleotide sequences encoding the VH-VL pair are selected from the paired nucleotide sequences set forth in SEQ ID NOs: 119 and 120, SEQ ID NOs: 113 and 114, SEQ ID NOs: 115 and 116, SEQ ID NOs: 117 and 118, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39.

38. The isolated polynucleotide sequence of claim 35, wherein said polynucleotide sequence comprises two polynucleotide sequences, a first polynucleotide sequence encoding the VH of the anti-IL-8 antibody and a second polynucleotide sequence encoding the VL of the anti-IL-8 antibody.

39. The isolated polynucleotide of claim 35, wherein said antibody comprises an IgG, an Fv, an scFv, an Fab, an F(ab′)₂, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody.

40. A vector comprising the polynucleotide sequence of claim 35.

41. A host cell comprising the vector of claim 40.

42. A method of producing an anti-IL-8 antibody having complementarity determining region (CDR) sequences as set forth in Table 1F, said method comprising the step of culturing the host cell of claim 41 under conditions conducive to expressing said vector in said host cell, and expressing said polynucleotide sequences comprised in said vector, thereby producing an anti-IL-8 antibody having complementarity determining region (CDR) sequences as set forth in:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109.

43. The method of claim 42, wherein each antibody comprises a heavy chain variable region (VH)—light chain variable region (VL) pair, and wherein the amino acid sequences of the VH-VL pair are selected from the paired sequences set forth in SEQ ID NOs: 8 and 9, SEQ ID NOs: 2 and 3, SEQ ID NOs: 4 and 5, SEQ ID NOs: 6 and 7, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35; or wherein the polynucleotide sequences encoding the heavy chain variable region (VH)—light chain variable region (VL) pair are selected from the paired sequences set for in SEQ ID NOs: 119 and 120, SEQ ID NOs: 113 and 114, SEQ ID NOs: 115 and 116, SEQ ID NOs: 117 and 118, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39.

44. The method of claim 42, wherein said antibody comprises an IgG, an Fv, an scFv, an Fab, an F(ab′)2, a minibody, a diabody, a triabody, a nanobody, a single domain antibody, a multi-specific antibody, a bi-specific antibody, a tri-specific antibody, a single chain antibodies, heavy chain antibodies, a chimeric antibodies, or a humanized antibody.

45. A method of inhibiting tumor or cancer formation or growth, or a combination thereof in a human subject in need, said method comprising a step of administering to said subject an anti-IL-8 antibody having complementarity determining region (CDR) sequences as set forth in Table 1F, wherein each antibody comprises a heavy chain variable region (VH) having heavy chain complementarity determining regions (HCDR) 1, HCDR2 and HCDR3, and a light chain variable region (VL) having light chain complementarity determining regions (LCDR) 1, LCDR2 and LCDR3, wherein said HCDR1, HCDR2, HCDR3, LCDR1, LCDR2 and LCDR3 for each of said antibody comprise the amino acid sequences as set forth in:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109
thereby inhibiting tumor formation or growth or a combination thereof in said subject.

46. The method of claim 45, wherein each antibody comprises a heavy chain variable region (VH)—light chain variable region (VL) pair, wherein the amino acid sequences of a VH-VL pair are selected from the paired sequences set forth in SEQ ID NOs: 8 and 9, SEQ ID NOs: 2 and 3, SEQ ID NOs: 4 and 5, SEQ ID NOs: 6 and 7, SEQ ID NOs: 10 and 11, SEQ ID NOs: 12 and 13, SEQ ID NOs: 14 and 15, SEQ ID NOs: 16 and 17, SEQ ID NOs: 18 and 19, SEQ ID NOs: 20 and 21, SEQ ID NOs: 22 and 23, SEQ ID NOs: 24 and 25, SEQ ID NOs: 26 and 27, SEQ ID NOs: 28 and 29, SEQ ID NOs: 30 and 31, SEQ ID NOs: 32 and 33, and SEQ ID NOs: 34 and 35.

47. A method of treating a human subject suffering from a disease, said method comprising the step of administering to said subject an anti-IL-8 antibody having complementarity determining region (CDR) sequences as set forth in Table 1F, wherein the disease comprises a cancer or tumor, a viral infection, or a disease associated with inflammation, or a combination thereof, and wherein each antibody comprises a heavy chain variable region (VH) having heavy chain complementarity determining regions (HCDR) 1, HCDR2 and HCDR3, and a light chain variable region (VL) having light chain complementarity determining regions (LCDR) 1, LCDR2 and LCDR3, wherein said HCDR1, HCDR2, HCDR3, LCDR1, LCDR2 and LCDR3 for each of said antibody comprise the amino acid sequences as set forth in:
(a) SEQ ID NO: 41, SEQ ID NO: 44, SEQ ID NO: 53, SEQ ID NO: 60, SEQ ID NO: 67, and SEQ ID NO: 76;
(b) SEQ ID NO: 81, SEQ ID NO: 84, SEQ ID NO: 53, SEQ ID NO: 93, SEQ ID NO: 100, and SEQ ID NO: 109;
(c) SEQ ID NO: 40, SEQ ID NO: 43, SEQ ID NO: 50, SEQ ID NO: 57, SEQ ID NO: 64, and SEQ ID NO: 73;
(d) SEQ ID NO: 40, SEQ ID NO: 43, SEQ ID NO: 51, SEQ ID NO: 58, SEQ ID NO: 65, and SEQ ID NO: 74;
(e) SEQ ID NO: 41, SEQ ID NO: 44, SEQ ID NO: 52, SEQ ID NO: 59, SEQ ID NO: 66, and SEQ ID NO: 75;
(f) SEQ ID NO: 40, SEQ ID NO: 43, SEQ ID NO: 50, SEQ ID NO: 57, SEQ ID NO: 68, and SEQ ID NO: 73;
(g) SEQ ID NO: 40, SEQ ID NO: 43, SEQ ID NO: 50, SEQ ID NO: 57, SEQ ID NO: 69, and SEQ ID NO: 73;
(h) SEQ ID NO: 41, SEQ ID NO: 45, SEQ ID NO: 53, SEQ ID NO: 61, SEQ ID NO: 70, and SEQ ID NO: 77;
(i) SEQ ID NO: 40, SEQ ID NO: 46, SEQ ID NO: 54, SEQ ID NO: 62, SEQ ID NO: 71, and SEQ ID NO: 78;
(j) SEQ ID NO: 42, SEQ ID NO: 47, SEQ ID NO: 53, SEQ ID NO: 63, SEQ ID NO: 72, and SEQ ID NO: 79;
(k) SEQ ID NO: 41, SEQ ID NO: 48, SEQ ID NO: 53, SEQ ID NO: 60, SEQ ID NO: 67, and SEQ ID NO: 76;
(l) SEQ ID NO: 41, SEQ ID NO: 49, SEQ ID NO: 53, SEQ ID NO: 60, SEQ ID NO: 67, and SEQ ID NO: 76;
(m) SEQ ID NO: 41, SEQ ID NO: 44, SEQ ID NO: 55, SEQ ID NO: 60, SEQ ID NO: 67, and SEQ ID NO: 76;
(n) SEQ ID NO: 41, SEQ ID NO: 44, SEQ ID NO: 56, SEQ ID NO: 60, SEQ ID NO: 67, and SEQ ID NO: 76;
(o) SEQ ID NO: 41, SEQ ID NO: 48, SEQ ID NO: 55, SEQ ID NO: 60, SEQ ID NO: 67, and SEQ ID NO: 76;
(p) SEQ ID NO: 41, SEQ ID NO: 48, SEQ ID NO: 56, SEQ ID NO: 60, SEQ ID NO: 67, and SEQ ID NO: 76;
(q) SEQ ID NO: 41, SEQ ID NO: 49, SEQ ID NO: 55, SEQ ID NO: 60, SEQ ID NO: 67, and SEQ ID NO: 76;
(r) SEQ ID NO: 41, SEQ ID NO: 49, SEQ ID NO: 56, SEQ ID NO: 60, SEQ ID NO: 67, and SEQ ID NO: 76;
(s) SEQ ID NO: 80, SEQ ID NO: 83, SEQ ID NO: 50, SEQ ID NO: 90, SEQ ID NO: 97, and SEQ ID NO: 106;
(t) SEQ ID NO: 80, SEQ ID NO: 83, SEQ ID NO: 51, SEQ ID NO: 91, SEQ ID NO: 98, and SEQ ID NO: 107;
(u) SEQ ID NO: 81, SEQ ID NO: 84, SEQ ID NO: 52, SEQ ID NO: 92, SEQ ID NO: 99, and SEQ ID NO: 108;
(v) SEQ ID NO: 80, SEQ ID NO: 83, SEQ ID NO: 50, SEQ ID NO: 90, SEQ ID NO: 101, and SEQ ID NO: 106;
(w) SEQ ID NO: 80, SEQ ID NO: 83, SEQ ID NO: 50, SEQ ID NO: 90, SEQ ID NO: 102, and SEQ ID NO: 106;
(x) SEQ ID NO: 81, SEQ ID NO: 85, SEQ ID NO: 53, SEQ ID NO: 94, SEQ ID NO: 103, and SEQ ID NO: 110;
(y) SEQ ID NO: 80, SEQ ID NO: 86, SEQ ID NO: 54, SEQ ID NO: 95, SEQ ID NO: 104, and SEQ ID NO: 111;
(z) SEQ ID NO: 82, SEQ ID NO: 87, SEQ ID NO: 53, SEQ ID NO: 93, SEQ ID NO: 105, and SEQ ID NO: 112;
(aa) SEQ ID NO: 81, SEQ ID NO: 88, SEQ ID NO: 53, SEQ ID NO: 93, SEQ ID NO: 100, and SEQ ID NO: 109;
(bb) SEQ ID NO: 81, SEQ ID NO: 89, SEQ ID NO: 53, SEQ ID NO: 93, SEQ ID NO: 100, and SEQ ID NO: 109;
(cc) SEQ ID NO: 81, SEQ ID NO: 84, SEQ ID NO: 55, SEQ ID NO: 93, SEQ ID NO: 100, and SEQ ID NO: 109;
(dd) SEQ ID NO: 81, SEQ ID NO: 84, SEQ ID NO: 56, SEQ ID NO: 93, SEQ ID NO: 100, and SEQ ID NO: 109;
(ee) SEQ ID NO: 81, SEQ ID NO: 88, SEQ ID NO: 55, SEQ ID NO: 93, SEQ ID NO: 100, and SEQ ID NO: 109;
(ff) SEQ ID NO: 81, SEQ ID NO: 88, SEQ ID NO: 56, SEQ ID NO: 93, SEQ ID NO: 100, and SEQ ID NO: 109;
(gg) SEQ ID NO: 81, SEQ ID NO: 89, SEQ ID NO: 55, SEQ ID NO: 93, SEQ ID NO: 100, and SEQ ID NO: 109; or
(hh) SEQ ID NO: 81, SEQ ID NO: 89, SEQ ID NO: 56, SEQ ID NO: 93, SEQ ID NO: 100, and SEQ ID NO: 109
thereby treating said disease in said subject.

48. The method of claim 47, wherein each antibody comprises a heavy chain variable region (VH)—light chain variable region (VL) pair, wherein the amino acid sequences encoding the VH-VL pair are selected from the paired sequences set forth in SEQ ID NOs: 119 and 120, SEQ ID NOs: 113 and 114, SEQ ID NOs: 115 and 116, SEQ ID NOs: 117 and 118, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39.

49. A method of inhibiting tumor or cancer formation or growth or a combination thereof in a human subject in need, said method comprising the step of administering to said subject a polynucleotide encoding an anti-IL-8 antibody comprising an antibody antigen-binding domain comprising a heavy chain variable region (VH) and a light chain variable region (VL) pair, wherein the polynucleotide sequences encoding the VH-VL pair are selected from the paired sequences set forth in SEQ ID NOs: 119 and 120, SEQ ID NOs: 113 and 114, SEQ ID NOs: 115 and 116, SEQ ID NOs: 117 and 118, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39, thereby inhibiting tumor formation or growth or a combination thereof in said subject.

50. A method of treating a human subject suffering from a disease, said method comprising the step of administering to said subject a polynucleotide encoding an anti-IL-8 antibody comprising an antibody antigen-binding domain comprising a heavy chain variable region (VH) and a light chain variable region (VL) pair, wherein the disease comprises a cancer or tumor or a viral infection or a disease associated with inflammation or a combination thereof, and wherein the polynucleotide sequences encoding the VH-VL pair are selected from the paired sequences set forth in SEQ ID NOs: 119 and 120, SEQ ID NOs: 113 and 114, SEQ ID NOs: 115 and 116, SEQ ID NOs: 117 and 118, SEQ ID NOs: 121 and 122, SEQ ID NOs: 123 and 124, SEQ ID NOs: 125 and 126, SEQ ID NOs: 127 and 128, SEQ ID NOs: 129 and 130, SEQ ID NOs: 131 and 132, SEQ ID NOs: 133 and 134, SEQ ID NOs: 135 and 136, SEQ ID NOs: 137 and 138, SEQ ID NOs: 139 and 140, SEQ ID NOs: 141 and 142, SEQ ID NOs: 36 and 37, and SEQ ID NOs: 38 and 39, thereby treating said disease in said subject.
